(19) United States
(12) Patent Application Publication
(10) Pub. No.: US 2020/0397894 A1
(43) Pub. Date: Dec. 24, 2020

(54) COMPOSITIONS AND METHODS FOR TREATING CANCER

(71) Applicant: University of Virginia Patent Foundation, Charlottesville, VA (US)

(72) Inventor: Tarek Abbas, Charlottesville, VA (US)

(73) Assignee: University of Virginia Patent Foundation, Charlottesville, VA (US)

(21) Appl. No.: 16/097,021

(22) PCT Filed: Apr. 27, 2017
(86) PCT No.: PCT/US2017/029859

§ 371 Date: Oct. 26, 2018

Related U.S. Application Data
(60) Provisional application No. 62/328,183, filed on Apr. 27, 2016.

(51) Int. Cl. A61K 39/395; A61K 31/519; C12N 15/113; A61K 31/506; A61K 31/437; A61K 31/44; A61K 38/20; A61P 35/00

(52) U.S. Cl. CPC A61K 39/3955; A61K 31/519; C12N 15/1137; A61P 35/00; A61K 31/437; A61K 31/44; A61K 38/2013; A61K 31/506

(57) ABSTRACT

Provided are compositions for inhibiting cellular proliferation comprising one or more CRL4^{CDT2} ubiquitin ligase inhibitors. Also provided are uses of the disclosed compositions to prepare medicaments, for treating and/or preventing diseases, disorders, and conditions, particularly for treating cancer, inducing apoptosis and/or rereplication in cells, inhibiting undesirable neddylation, overcoming vemurafenib-resistance in cells, treating melanoma, breast cancer, head and neck squamous carcinoma cell (HNSCC) cancer, a solid tumor, hepatocellular carcinoma, colorectal cancer, a non-small-cell lung cancer, serous ovarian cancer, papillary thyroid carcinoma, or ameloblastoma, and pharmaceutical compositions comprising the same.

### CROSS-REFERENCE TO RELATED APPLICATION

This application claims the benefit of U.S. Provisional Patent Application Ser. No. 62/328,183, filed Apr. 27, 2016, the disclosure of which is incorporated herein by reference in its entirety.

### GRANT STATEMENT

This invention was made with government support under Grant Nos. CA140774 and CA044579, awarded by The National Institutes of Health. The government has certain rights in the invention.

### BACKGROUND

Melanoma is an aggressive cancer affecting approximately 80,000 patients per year in the USA alone, with poor prognosis in the metastatic stage (Balch et al., 2009). It is the sixth most common fatal malignancy accounting for about 4% of all cancer-related deaths (Siegel et al., 2012). At the molecular level, activating mutations in the serine/threonine kinase BRAF (p.V600E) or NRAS (mostly p.Q61R or p.Q61K) occur in a majority (60-70%) of cases (Davies et al., 2002; Davies & Samuels, 2010). Both oncogenes activate the classical mitogen-activated protein kinase (MAPK) pathway, but NRAS additionally activates the phosphatidyl-inositol 3-kinase (PI3K) pro-survival pathway. Additional mutations in NF1, KIT, GNAQ, and GNA11 activate the MAPK pathway in certain subtypes of melanoma (Solus & Kraft, 2013). The loss of tumor suppressor genes such as PTEN, CDKN2A (encoding p16) and TP53, also contribute to melanomagenesis (Solus & Kraft, 2013).

The recent development of inhibitors of oncogenic BRAF such as vemurafenib and dabrafenib, has offered significant opportunities for the treatment of at least a subset of melanoma patients (Chapman et al., 2011; Flaherty et al., 2010; Hersey et al., 2009; Sosman et al., 2012). However, there is currently no effective treatment for NRAS or other non-BRAF melanomas. This and the development of therapeutic resistance present significant challenges that require intensified search of alternative therapeutic approaches and new molecular targets and chemical inhibitors that can exert anti-melanoma activity and can operate irrespective of the BRAF and/or NRAS mutational status.

Polyubiquitylation leading to proteolytic degradation by the 26S proteasome is involved in all aspects of cell physiology. The highly coordinated process ensures the selective and timely turnover of proteins thereby controlling cellular activity and maintaining cell and tissue homeostasis (Glickman & Ciechanover, 2002). The cullin 4 RING E3 ubiquitin ligase (CRL4) is a master regulator of genome stability and orchestrates a variety of physiological processes, particularly those related to chromatin regulation (Jackson & Xiong, 2009). Along with the substrate receptor CDT2 (also known as DCAF2 and DTL/RAMP), the CRL4^{CDT2} ligase promotes the ubiquitin-dependent degradation of multiple proteins essential for cell cycle progression as well as for DNA replication and repair (Abbas & Dutta, 2011; Abbas et al., 2013). Recent evidence indicates that one of the main functions of CRL4^{CDT2} is to prevent re-initiation of DNA replication (rereplication), both during S-phase of the cell cycle and following DNA damage, through the ubiquitylation and degradation of the replication licensing protein CDT1 (unrelated to CDT2), the CDK inhibitor p21, and the histone methyltransferase SET8 (Abbas & Dutta, 2011; Abbas et al., 2013). DNA rereplication is deleterious to cells and promotes cellular senescence and apoptosis due to replication fork stalling and the accumulation of toxic replication intermediates.

Cullin-dependent E3 ligases, including CRL4, are activated by NEDD8 modification, which is catalyzed by an enzyme cascade system similar to ubiquitylation (Merlet et al., 2009). Pevonedistat (MLN4924), an inhibitor of the NEDD8-activating enzyme (NAE), induces cytotoxicity in a variety of cancer cell types in vitro and in preclinical mouse models (Jazaeri et at, 2013; Lin et al., 2010; Soucy et al., 2009; Wei et al., 2012). It is currently in clinical trials for hematologic (ClinicalTrials Identifiers NCT00722488 and NCT00911066) and solid malignancies including melanoma (ClinicalTrials Identifier NCT01011530), but its effects on melanoma cells have not been thoroughly examined. There is also little to no preclinical data on pevonedistat efficacy in the context of the various genetic mutations associated with melanoma or resistance to front line therapies (Garcia et al., 2014; Tan et al., 2013). Furthermore, although pevonedistat inhibits the NFκB, AKT and the mTOR signal transduction pathways in addition to cullin-mediated signaling (Godbersen et al., 2014; Gu et al., 2014; Li et al., 2014a; Li et al., 2014b; Lin et al., 2010; Milhollen et al., 2011; Milhollen et al., 2010; Soucy et al., 2009), it is not clear which of these activities contributes to its anti-tumor activity.

The presently disclosed subject matter thus provides compositions and methods useful for treating melanoma.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides compositions for inhibiting cellular proliferation. In some embodiments, the compositions comprise an effective amount of an inhibitor of a cullin-based CRL4^{CDT2} ubiquitin ligase biological activity, optionally wherein the inhibitor of the cullin-based CRL4^{CDT2} ubiquitin ligase biological activity is an inhibitor of a NEDD8 activating enzyme (NAE). In some embodiments, the inhibitor of a cullin-based CRL4^{CDT2} ubiquitin ligase biological activity is pevonedistat, a pharmaceutically acceptable salt and/or solvate thereof, or any combination thereof. In some embodiments, the composition further comprises at least one second therapeutic agent selected from the group consisting of a BRAF inhibitor, an MEK inhibitor, an anti-CRL4^{CDT2} ubiquitin ligase inhibitory nucleic acid, an anti-p21 inhibitory nucleic acid, an anti-CDT1 inhibitory nucleic acid, an anti-SET8 inhibitory nucleic acid, an anti-geminin inhibitory nucleic acid, an anti-CDKN1A inhibitory nucleic acid, an anti-EMI1 inhibitory nucleic acid, or any combination thereof. In some embodiments, the BRAF inhibitor is selected from the group consisting of vemurafenib or a pharmaceutically acceptable salt and/or solvate thereof, dabrafenib or a pharmaceutically acceptable salt and/or solvate thereof, and sorafenib or a pharmaceutically acceptable salt and/or solvate thereof, or any combination thereof. In some embodiments, the MEK inhibitor is trametinib or a pharmaceutically acceptable salt and/or solvate thereof, or any combination thereof. In some embodiments, the anti-CRL4^{CDT2} ubiquitin ligase inhibitory nucleic acid comprises SEQ ID NO: 2, SEQ ID NO: 15, or SEQ ID NO: 16; and/or the anti-p21 inhibitory nucleic acid comprises SEQ ID NO: 5; and/or the anti-CDT1 inhibitory nucleic acid comprises SEQ ID NO: 3; and/or the anti-SET8 inhibitory nucleic acid comprises SEQ ID NO: 4 or SEQ ID NO: 17; and/or the anti-geminin inhibitory nucleic acid comprises SEQ ID NO: 6; and/or the anti-CDKN1A inhibitory nucleic acid comprises SEQ ID NO: 19; and/or the anti-EMI1 inhibitory nucleic acid comprises SEQ ID NO: 7 or SEQ ID NO: 8.

In some embodiments, the presently disclosed compositions further comprise a delivery vehicle associated with, conjugated to, and/or encapsulating the inhibitor of a cullin-based CRL4^{CDT2} ubiquitin ligase biological activity and/or any of the at least one second therapeutic agents, if present. In some embodiments, the delivery vehicle comprises a liposome, a microparticle, or a nanoparticle, optionally wherein the liposome, microparticle, or nanoparticle is designed to be biodegradable in a subject.

In some embodiments, the presently disclosed compositions further comprise one or more pharmaceutically acceptable excipients, diluents, and/or carriers, optionally wherein the one or more pharmaceutically acceptable excipients, diluents, and/or carriers are pharmaceutically acceptable for use in a human.

In some embodiments, the presently disclosed compositions are formulated for oral administration, intravenous administration, intramuscular administration, intrathecal administration, cutaneous administration, topical administration, transdermal administration, systemic administration, subcutaneous administration, sublingual administration, buccal administration, ocular administration, otic administration, nasal administration, inhalation, nebulization, or any combination thereof.

In some embodiments, the presently disclosed subject matter also provides for uses of compositions comprising one or more inhibitors of a cullin-based CRL4^{CDT1} ubiquitin ligase biological activity, optionally in combination with one or more inhibitors of a NEDD8 activating enzyme (NAE) in the preparation of medicaments for the treatment of cancer. In some embodiments, the cancer is associated with a cullin-based CRL4^{CDT2} ubiquitin ligase biological activity. In some embodiments, the cancer is selected from the group consisting of melanoma, breast cancer, head and neck squamous carcinoma cell (HNSCC) cancer, a solid tumor, hepatocellular carcinoma, colorectal cancer, non-small-cell lung cancer, serous ovarian cancer, papillary thyroid carcinoma, and ameloblastoma. In some embodiments, the inhibitor of a cullin-based CRL4^{CDT2} ubiquitin ligase biological activity comprises pevonedistat, a pharmaceutically acceptable salt and/or solvate thereof, or any combination thereof. In some embodiments, the composition further comprises vemurafenib, emurafenib, dabrafenib, trametinib, cobimetinib, a pharmaceutically acceptable salt and/or solvate thereof, or any combination thereof. In some embodiments, the composition further comprises vemurafenib, a pharmaceutically acceptable salt and/or solvate thereof, or any combination thereof. In some embodiments, the cell from the melanoma, breast cancer, head and neck squamous carcinoma cell (HNSCC) cancer, solid tumor, hepatocellular carcinoma, colorectal cancer, non-small-cell lung cancer, serous ovarian cancer, papillary thyroid carcinoma, or ameloblastoma is present in a subject.

In some embodiments, the presently disclosed subject matter provides methods for treating and/or preventing diseases, disorders, and/or conditions associated with CRL4^{CDT2} one or more ubiquitin ligase biological activities. In some embodiments, the methods comprise administering to a subject in need thereof one or more compositions as disclosed herein in an effective amount and via a route sufficient for treating and/or treating at least one symptom of the disease, disorder, or condition. In some embodiments, the CRL4^{CDT2} ubiquitin ligase biological activity is present in a CDT2-overexpressing cell, optionally a CDT2-overexpressing tumor cell. In some embodiments, the disease, disorder, or condition is cancer, optionally wherein the cancer is selected from the group consisting of melanoma, breast cancer, head and neck squamous carcinoma cell (HNSCC) cancer, a solid tumor, hepatocellular carcinoma, colorectal cancer, non-small-cell lung cancer, serous ovarian cancer, papillary thyroid carcinoma, and ameloblastoma.

The presently disclosed subject matter also provides methods for treating cancer. In some embodiments, the cancer is selected from the group consisting of melanoma, breast cancer, head and neck squamous carcinoma cell (HNSCC) cancer, a solid tumor, hepatocellular carcinoma, colorectal cancer, non-small-cell lung cancer, serous ovarian cancer, papillary thyroid carcinoma, and ameloblastoma. In some embodiments, the methods comprise administering to a subject in need thereof one or more compositions as disclosed herein in an amount sufficient to inhibit a CRL4^{CDT2} ubiquitin ligase biological activity.

In some embodiments, the presently disclosed subject matter also provides methods for treating and/or preventing diseases, disorders, and/or conditions associated with undesirable cullin signaling. In some embodiments, the methods comprise administering to a subject one or more composition as disclosed herein in an effective amount and via a route sufficient for treating and/or preventing at least one symptom of the disease, disorder, or condition. In some embodiments, the cullin signaling is present in a cell that overexpresses a cullin-based CRL4^{CDT2} ubiquitin ligase, optionally wherein the cell is a tumor cell or a cancer cell. In some embodiments, the disease, disorder, or condition is cancer, optionally wherein the cancer is selected from the group consisting of melanoma, breast cancer, head and neck squamous carcinoma cell (HNSCC) cancer, a solid tumor, hepatocellular carcinoma, colorectal cancer, non-small-cell lung cancer, serous ovarian cancer, papillary thyroid carcinoma, and ameloblastoma.

In some embodiments, the presently disclosed subject matter also provides methods for inducing apoptosis and/or rereplication in cells. In some embodiments, the methods comprise contacting a cell with an effective amount of a composition as disclosed herein. In some embodiments, the cell overexpresses a cullin-based CRL4^{CDT2} ubiquitin ligase. In some embodiments, the cell is a tumor cell or a cancer cell. In some embodiments, the tumor cell or the cancer cell is selected from the group consisting of a melanoma cell, a breast cancer cell, a head and neck squamous carcinoma cell (HNSCC) cancer cell, a solid tumor cell, a hepatocellular carcinoma cell, a colorectal cancer cell, anon-small-cell lung cancer cell, a serous ovarian cancer cell, a papillary thyroid carcinoma cell, and an ameloblastoma cell.

In some embodiments, the presently disclosed subject matter also provides methods for treating cancer. In some embodiments, the cancer is selected from the group consisting of melanoma, breast cancer, head and neck squamous carcinoma cell (HNSCC) cancer, a solid tumor, hepatocellular carcinoma, colorectal cancer, non-small-cell lung cancer, serous ovarian cancer, papillary thyroid carcinoma, and ameloblastoma, the method comprising administering to a subject in need thereof a composition comprising an effective amount of a composition as disclosed herein. In some embodiments, the composition comprises pevonedistat or a pharmaceutically acceptable salt and/or solvate thereof, and vemurafenib or a pharmaceutically acceptable salt and/or solvate thereof.

In some embodiments, the presently disclosed subject matter also provides methods for inhibiting undesirable neddylation. In some embodiments, the methods comprise contacting a cell in which the undesirable neddylation is occurring or will occur with an effective amount of pevonedistat, vemurafenib, pharmaceutically acceptable salts and/or solvates thereof, or any combination thereof. In some embodiments, the cell is present within a subject, which in some embodiments is a human subject. In some embodiments, the cell is a tumor cell and/or a cancer cell, which in some embodiments is selected from the group consisting of a melanoma cell, a breast cancer cell, a head and neck squamous carcinoma cell (HNSCC) cancer cell, a solid tumor cell, a hepatocellular carcinoma cell, a colorectal cancer cell, anon-small-cell lung cancer cell, a serous ovarian cancer cell, a papillary thyroid carcinoma cell, and an ameloblastoma cell.

The presently disclosed subject matter also provides methods for overcoming vemurafenib-resistance in cells. In some embodiments, the method comprising contacting a vemurafenib-resistant cell with an effective amount of an inhibitor of cullin-based CRL4^{CDT2} ubiquitin ligase biological activity, optionally wherein the inhibitor of cullin-based CRL4^{CDT2} ubiquitin ligase biological activity comprises pevonedistat, a pharmaceutically acceptable salt and/or solvate thereof, or any combination thereof. In some embodiments, the cell is a tumor cell and/or a cancer cell, optionally a melanoma cell or a colorectal cell.

In some embodiments, the presently disclosed subject matter also provides methods for treating melanoma, breast cancer, head and neck squamous carcinoma cell (HNSCC) cancer, a solid tumor, hepatocellular carcinoma, colorectal cancer, a non-small-cell lung cancer, serous ovarian cancer, papillary thyroid carcinoma, or ameloblastoma. In some embodiments, the methods comprise contacting a cell from the melanoma, breast cancer, head and neck squamous carcinoma cell (HNSCC) cancer, solid tumor, hepatocellular carcinoma, colorectal cancer, non-small-cell lung cancer, serous ovarian cancer, papillary thyroid carcinoma, or ameloblastoma with an effective amount of a composition as disclosed herein. In some embodiments, the composition comprises pevondestat, a pharmaceutically acceptable salt and/or solvate thereof, or any combination thereof.

In some embodiments, the presently disclosed subject matter also provides methods for treating tumors and/or cancers in a subject. In some embodiments, the methods comprise identifying a subject having a tumor and/or a cancer associated with CDT2 overexpression; administering to the subject a therapeutic agent comprising a composition as disclosed herein; and administering to the subject radiation therapy before, during, and/or after administering to the subject the therapeutic agent. In some embodiments, the tumor and/or the cancer is selected from the group consisting of melanoma, breast cancer, head and neck squamous carcinoma cell (HNSCC) cancer, a solid tumor, hepatocellular carcinoma, colorectal cancer, a non-small-cell lung cancer, serous ovarian cancer, papillary thyroid carcinoma, and ameloblastoma.

In some embodiments, the presently disclosed methods further comprise treating the subject with at least one additional anti-cancer therapy. In some embodiments, the at least one additional anti-cancer therapy is selected from the group consisting of radiotherapy, chemotherapy, immunotherapy, surgery, and combinations thereof. In some embodiments, the at least one additional anti-cancer therapy comprises administering vemurafenib, dabrafenib, trametinib, cobimetinib, a pharmaceutically acceptable salt and/or solvate thereof, or any combination thereof to the subject. In some embodiments, the at least one additional anti-cancer therapy comprises administering vemurafenib, a pharmaceutically acceptable salt and/or solvate thereof, or any combination thereof, to the subject in need thereof. In some embodiments, the at least one additional anti-cancer therapy comprises administering ipilimumab, pembrolizumab, nivolumab, interleukin-2 (IL-2), a pharmaceutically acceptable salt and/or solvate thereof, or any combination thereof, or any combination thereof to the subject. In some embodiments, the at least one additional anti-cancer therapy comprises administering to the subject at least one second therapeutic agent selected from the group consisting of a BRAF inhibitor, an MEK inhibitor, an anti-CRL4^{CDT2} ubiquitin ligase inhibitory nucleic acid, an anti-p21 inhibitory nucleic acid, an anti-CDT1 inhibitory nucleic acid, an anti-SET8 inhibitory nucleic acid, an anti-geminin inhibitory nucleic acid, an anti-CDKN1A inhibitory nucleic acid, an anti-EMI1 inhibitory nucleic acid, or any combination thereof. In some embodiments, the BRAF inhibitor is selected from the group consisting of vemurafenib or a pharmaceutically acceptable salt and/or solvate thereof, dabrafenib or a pharmaceutically acceptable salt and/or solvate thereof, and sorafenib or a pharmaceutically acceptable salt and/or solvate thereof, or any combination thereof; and/or the MEK inhibitor is trametinib or a pharmaceutically acceptable salt and/or solvate thereof, or any combination thereof; and/or the anti-CRL4^{CDT2} ubiquitin ligase inhibitory nucleic acid comprises SEQ ID NO: 2, SEQ ID NO: 15, or SEQ ID NO: 16; and/or the anti-p21 inhibitory nucleic acid comprises SEQ ID NO: 5, and/or the anti-CDT inhibitory nucleic acid comprises SEQ ID NO: 3; and/or the anti-SET8 inhibitory nucleic acid comprises SEQ ID NO: 4 or SEQ ID NO: 17; and/or the anti-geminin inhibitory nucleic acid comprises SEQ ID NO: 6; and/or the anti-CDKN1A inhibitory nucleic acid comprises SEQ ID NO: 19, and/or the anti-EMI1 inhibitory nucleic acid comprises SEQ ID NO: 7 or SEQ ID NO: 8. In some embodiments, the at least one additional anti-cancer therapy is administered to the subject in a separate composition and in some embodiments the at least one additional anti-cancer therapy are present in the same composition.

In some embodiments, the presently disclosed subject matter also provides pharmaceutical compositions comprising the presently disclosed compositions and at least one pharmaceutically acceptable carrier, diluent, and/or excipient. In some embodiments, the presently disclosed pharmaceutical compositions are formulated for use in one or more of the presently disclosed methods. In some embodiments, the pharmaceutical composition is pharmaceutically acceptable for use in a human.

In some embodiments, the cancers that can be treated using the compositions and methods of the presently disclosed subject matter include, but are not limited to, melanoma, glioblastoma, invasive breast cancer, squamous cell lung carcinoma, hepatocellular carcinoma, gastric adenocarcinoma, and cervical squamous cell carcinoma.

In some embodiments, the melanoma is cutaneous melanoma.

CRL4^{CDT2} inactivation in melanoma induces p21- and Set8-dependent rereplication.

In some embodiments, pevonedistat inhibits cancer cell proliferation. In some embodiments, the cancer cell is a melanoma cell. In some embodiments, the cancer cell over expresses CDT2.

In some embodiments, pevonedistat inhibits melanoma in vitro and in vivo through SET8 and p21. In some embodiments, it inhibits melanoma cells through the induction of rereplication and senescence.

In some embodiments, increased CDT2 expression renders melanoma cells susceptible to pevonedistat-induced rereplication.

In some embodiments, pevonedistat inhibits cullin signaling.

In some embodiments, cancer cells, but not immortalized cells that are not tumorigenic, are sensitive to pevonedistat. In some embodiments, pevonedistat induces rereplication in melanoma cells but not melanocytes. In some embodiments, pevonedistat induces growth arrest in melanoma cells, but not normal cells. In some embodiments, the normal cells are melanocytes. In some embodiments, the melanocytes are immortalized melanocytes but are not transformed. In some embodiments, the growth arrest is permanent (i.e., irreversible).

In some embodiments, the effects of pevonedistat are independent of BRAF mutational status. In some embodiments, the effects of pevonedistat are independent of NRAS mutational status.

In some embodiments, pevonedistat treatment is coupled with additional anti-cancer chemotherapeutic agents. In some embodiments, the agent is vemurafenib. In some embodiments, pevonedistat synergizes with vemurafenib. In some embodiments, the combination therapy inhibits vemurafenib-resistant cells.

In some embodiments, pevonedistat treatment is useful for treating vemurafenib-relapsed subjects.

One of ordinary skill in the art will appreciate that neddylation inhibitors other than pevonedistat can be used to practice the presently disclosed subject matter.

One of ordinary skill in the art will appreciate that inhibitors such as pevonedistat are also useful in treating other tumors that exhibit increased levels or activity of CDT2.

The presently disclosed subject matter further provides for the use of biologically active analogs and derivatives of the compounds of the presently disclosed subject matter, wherein the activity of the analogs and derivatives is similar to the compound as disclosed herein.

In some embodiments, a treatment regimen can be developed based on detecting cancer cells overexpressing CDT2 in a subject. In some embodiments, the cancer is melanoma.

In some embodiments, the presently disclosed subject matter provides compositions and methods for treating cancer by inhibiting the expression, levels, or activity of CDT2 in the cancer cells, wherein the subject has a cancer that expresses high levels of CDT2 or high activity of CDT2. The presently disclosed subject matter provides compositions and methods for determining setting up such treatment regimens.

These and other aspects and embodiments which will be apparent to those of skill in the art upon reading the present disclosure provide compositions and methods useful for diagnosing, prognosing, monitoring, and treating human cancers.

### BRIEF DESCRIPTION OF THE DRAWINGS

FIG. 1 is a graph showing that depletion of DM93 melanoma cells of CDT2 inhibited cell proliferation. Data represent the average of three independent experiments±S.D. (error bars). Diamonds: si-GL2 (negative control siRNA; SEQ ID NO: 1); Squares: si-CDT2 (siRNA targeted to CDT2; SEQ ID NO: 2). Inset: Western blot of cell lysates extracted from transfected DM93 cells and probed with the indicated antibodies showing lack of expression of CDT2 protein resulting from si-CDT2 targeting.

FIG. 2 is a series of immunoblots of protein lysates extracted from the indicated melanoma cell lines after transfection with si-GL2 control (SEQ ID NO: 1) or with si-CDT2 (SEQ ID NO: 2) for 72 hours. Mut:BRAF: cell lines that were negative (mutant) with respect to BRAF; Mut-NRAS: cell lines that were negative (mutant) with respect to NRAS; wt-BRAF/NRAS: cell lines that were wild-type with respect to both BRAF and NRAS. CDT2: CDT2 protein. CDT1: CDT1 protein. SET8: SET8 protein, p21: p21 protein, p-CHK1: phosphorylated CHK-1 protein, p-CHK2: phosphorylated CHK-2 protein. γH2AX: phosphorylated H2AX protein. H2AX: H2AX protein. Tubulin: tubulin protein (loading control).

FIG. 3 is a bar graph showing the percentage of the indicated melanoma cell lines undergoing senescence 96 hours following transfection with control (si-GL2 (SEQ ID NO: 1); white bars) or CDT2-targeted siRNA (si-CDT2 (SEQ ID NO: 2); black bars). Data represent the average of three independent experiments±S.D. (−S.D. error bars not depicted). CDKN2A^{+/+}: cell lines that were wild-type with respect to CDKN2A/p16; CDKN2A^{-/-}: cell lines that were negative (mutant) with respect to CDKN2A/p16. ns: not significant; *p<0.05; **p<0.01; ***p<0.001.

FIGS. 4-6 present the results of experiments showing that CDT2 depletion induced SET8 and p21-dependent rereplication and senescence. See also FIGS. 7-9.

FIG. 4 shows the results of immunoblotting of lysates extracted from DM93 cells transfected with the indicated siRNAs (si-GL2: SEQ ID NO: 1; si-CDT2: SEQ ID NO: 2; si-CDT1: SEQ ID NO: 3; si-p21: SEQ ID NO: 5; si-SET8: SEQ ID NO: 4) and probed with antibodies that detected the indicated proteins. The last three lanes show lysates from cells that were transfected with si-CDT2 (SEQ ID NO: 2) in addition to si-CDT1 (SEQ ID NO: 3), si-p21 (SEQ ID NO: 5), or si-SET8 (SEQ ID NO: 4), respectively.

FIG. 5 is a bar graph demonstrating the extent of rereplication observed in cells treated as in FIG. 4 and pulsed with BrdU for one hour prior to fixation and FACS analysis. The last three bars show lysates from cells that were transfected with si-CDT2 (SEQ ID NO: 2) in addition to si-CDT1 (SEQ ID NO: 3), si-p21 (SEQ ID NO: 5), or si-SET8 (SEQ ID NO: 4), respectively. Data represent the average of three independent experiments±S.D. *p<0.05.

FIG. 6 is a bar graph showing the percentage of senescent DM93 cells (determined by β-gal staining) treated with the indicated siRNA (si-GL2: SEQ ID NO: 1; si-CDT2: SEQ ID NO: 2; si-CDT1: SEQ ID NO: 3; si-p21: SEQ ID NO: 5; si-SET8: SEQ ID NO: 4). The last three bars show lysates from cells that were transfected with si-CDT2 (SEQ ID NO: 2) in addition to si-CDT1 (SEQ ID NO: 3), si-p21 (SEQ ID NO: 5), or si-SET8 (SEQ ID NO: 4), respectively. Data represent the average of three independent experiments±S.D. **p<0.01; ***p<0.001.

FIG. 7 is a bar graph of DM93 cells showing the distribution of cells in various cell cycle stages following transfection with the indicated siRNAs (si-GL2: SEQ ID NO: 1; si-CDT2: SEQ ID NO: 2; si-CDT: SEQ ID NO: 3; si-p21: SEQ ID NO: 5; si-SET8: SEQ ID NO: 4). For each group of eight, the last three bars show lysates from cells that were transfected with si-CDT2 (SEQ ID NO: 2) in addition to si-CDT1 (SEQ ID NO: 3), si-p21 (SEQ ID NO: 5), or si-SET8 (SEQ ID NO: 4), respectively.

FIG. 8 is a series of immunoblots of VMM39 cell extract following transfection with the indicated siRNAs. The last three lanes show lysates from cells that were transfected with si-CDT2 (SEQ ID NO: 2) in addition to si-CDT1 (SEQ ID NO: 3), si-p21 (SEQ ID NO: 5), or si-SET8 (SEQ ID NO: 4), respectively. Tubulin was a loading control.

FIG. 9 is a bar graph showing the extent of rereplication in VMM39 cells as determined by FACS analysis. The last three bars show lysates from cells that were transfected with si-CDT2 (SEQ ID NO: 2) in addition to si-CDT1 (SEQ ID NO: 3), si-p21 (SEQ ID NO: 5), or si-SET8 (SEQ ID NO: 4), respectively. Data represent the average of three independent experiments±S.D. (error bars). p-values were calculated using Student's t-test. *p<0.05**p<0.01.

FIG. 10 is a bar graph showing the extent of rereplication as determined by FACS for DM93, VMM39, Cal27, and U2OS cells transfected with a control siRNA (si-GL2; SEQ ID NO: 1) or an siRNA that targeted geminin (si-Geminin; SEQ ID NO: 6). Data represent the average of three independent experiments±S.D. (−S.D. error bars not depicted). ***p<0.001; ns: not significant.

FIGS. 11 and 12 show that a CRL4^{CDT2} insensitive mutant of CDT1 did not cause more rereplication than wild-type CDT1 in melanoma cells. FIG. 11 is a Western blot of control DM39 cells or ectopically expressing the indicated CDT1 proteins from retroviruses. Tubulin was a loading control. FIG. 12 is a bar graph depicting the extent of rereplication induced in DM93 cells by retroviruses expressing the indicated proteins. Data represent the average of three independent experiments±S.D. (−S.D. error bars not depicted). See also FIG. 13. PMX: negative control empty retrovirus. wt-CDT1: wild-type CDT1. CDT1^{ΔPIP}. CRL4^{CDT2}-resistant CDT mutant. CDT1^{ΔCY}. SCF^{SKP2}-resistant CDT1 mutant. **p<0.01, calculated using Student's t-test.

FIG. 13 is a bar graph depicting the relative expression of wild type and the indicated mutant CDT1 mRNAs, normalized to β-actin mRNA and expressed relative to wild-type CDT1 mRNA following retroviral transduction of DM93 cells. Data represent the average of three independent experiments±S.D. (−S.D. error bars not depicted).

FIG. 14 is an immunoblot of DM93 and VMM39 cell extract following transduction with retrovirus expressing the indicated proteins. Tubulin was a loading control. pMSCV: extract of cells transfected with an empty virus (negative control); FLAG-Set8: extract of cells transfected with a retrovirus encoding an N-terminal FLAG-tagged Set8 protein; FLAG-Set8^{ΔPIP}: extract of cells transfected with a retrovirus encoding an N-terminal FLAG-tagged mutant Set8 protein that cannot associate with PCNA and is thus resistant to CRL4^{CDT2} degradation (Abbas et al., 2010); FLAG-Set8^{ΔPIP-CD}: extract of cells transfected with a retrovirus encoding an N-terminal FLAG-tagged mutant Set8 protein that is catalytically inactive; FLAG-p21^{ΔPIP}: extract of cells transfected with a retrovirus encoding an N-terminal FLAG-tagged mutant p21 protein that is resistant to CRL4^{CDT2} degradation (Abbas et al., 2008); Set8^{ΔPIP}+p21^{ΔPIP}: extract of cells transfected with a retrovirus encoding a CRL4^{CDT2}-resistant Set8 protein and a retrovirus encoding a CRL4^{CDT2}-resistant p21 protein. Asterisk: cross-reactive band.

FIG. 15 is a bar graph displaying the extent of rereplication induced in DM93 cells (white bars) or VMM39 cells (black bars) treated as in FIG. 14 and as determined by FACS analysis. Data represent the average of three independent experiments±S.D. (−S.D. error bars not depicted). ***p<0.001. See also FIGS. 13, 16, and 17.

FIGS. 16 and 17 are a Western blot showing the expression of mutant SET8 proteins (FIG. 16) and a bar graph showing the % of cells undergoing rereplication (FIG. 17) in DM93 and VMM39 cells transduced with lower titer for the catalytically active SET8 (SET8^{ΔPIP}), but with higher titer of catalytically inactive protein (SET8^{ΔPIP-CD}) or empty vector (pMSCV). Data represent the average of three independent experiments±S.D. (−S.D. error bars not depicted). p values were calculated using Student's t-test. ***p<0.001.

FIG. 18 is a series of representative images of DM93 treated as in FIG. 14 and stained with β-gal (darker staining) to monitor senescence.

FIG. 19 is a bar graph displaying the extent of senescence induced in DM93 (white bar) and VMM39 (black bar) following the expression of the indicated proteins. Data represent the average of three independent experiments±S.D. (−S.D. error bars not depicted).

FIG. 20 is a series of Western blots of DM93 cell lysates following treatment with the indicated doses of pevonedistat analyzed 24 hours post-treatment and showing that pevonedistat induced dose-dependent increase in cullin-dependent substrates CDT2, CDT1, p21, and p27. Tubulin was a loading control. See also FIG. 22.

FIG. 21 is the same as FIG. 20 except that cells were treated with 1 μM pevonedistat and harvested at the indicated time points following treatment. ^{nd}Cullin 3: neddylated cullin 3; Cullin 3: undeddylated cullin 3; H4K20me-1: mon-methylated histone H4K20; p-CHK1: phosphorylated CHK-1 protein. p-CHK2: phosphorylated CHK-2 protein. P-CDC2: phosphoryated cell division control 2 (CDC); γH2AX: phosphorylated H2AX protein. C-PARP: C-terminal cleavage fragment (“p85” fragment) of poly(ADP-ribose) polymerase (PARP). Tubulin: tubulin protein (loading control).

FIG. 22 is an immunoblot of cell lysates extracted from DM93 cells treated with 1 μM pevonedistat for 12 hours followed by treatment with cycloheximide (CHX) for the indicated time points. Immunoblotting with anti-H4K20 mono-(H4K20-me1), di-(H4K20-me2), and tri-methylation (H4K20-me3)-specific antibodies and with anti-tubulin is also shown. ^{nd}Cul 3: neddylated cullin 3; Cul 3: undeddylated cullin 3.

FIG. 23 is a bar graph displaying the percentage of the indicated lines with greater than G2/M DNA content following treatment with 1 μM pevonedistat as analyzed by FACS at 24 hours (hatched bars) or 72 hours (black bars). FIG. 23 shows that pevonedistat induced rereplication in a panel of melanoma cell lines with various mutations. White bars: DMSO (negative) control. Data represent the average of three independent experiments±S.D. (−S.D. error bars not depicted).

FIG. 24 is a bar graph depicting the extent of rereplication as determined by FACS analysis of cells retrovirally overexpressing a wild-type CDT2 protein (Flag-CDT2) or a mutant CDT2 protein (CDT2^{246A}; cannot bind DDB1 and was thus incapable of assembling functioning CRL4^{CDT2} ligase). Data represent the average of three independent experiments±S.D. (−S.D. error bars not depicted). *p<0.05; **p<0.01. PMSCV (empty virus): white bars; Flag-CDT2 protein: hatched bars; FLAG-CDT^{246A} protein: black bars.

FIG. 25 is a bar graph displaying the percentage of the indicated melanoma lines that underwent senescence following treatment with 1 μM pevonedistat (black bars) and analyzed 96 hours following treatment. The white bars represent the negative controls (DMSO). Data represent the average of three independent experiments±S.D. (−S.D. error bars not depicted).

FIG. 26 shows the results of immunoblotting DM93 cell protein extracts for the indicated proteins treated with pevonedistat and harvested according the schematic time line of drug addition and withdrawal (wash out (WO) at 4, 8, 12, and 24 hours, with harvesting at the time points listed above the Western blot; see Top). Tubulin was loading control.

FIG. 27 is a bar graph displaying the percentage of DM93 cells shown in FIG. 26 undergoing rereplication as determined by propidium iodide (PI) staining and FACS analysis. Data represent the average of three independent experiments±S.D.

FIG. 28 is a bar graph displaying the percentage of PIG3V cells shown in FIG. 29 in various phases of the cell cycle (G1 phase—white bars; S phase—hatched bars; and G2/M phrase—black bars) as determined by PI staining and FACS analysis.

FIG. 29 is an immunoblot for the indicated proteins of PIG3V cells treated as in FIG. 26 (wash out (WO) at 4, 8, 12, and 24 hours, with harvesting at the time points listed above the Western blot). Tubulin was a loading control.

FIG. 30 is a Western blot of proteins extracted from DM93 cells transfected with the indicated siRNAs and treated with pevonedistat for 24 hours. Negative (DMSO) controls also included.

FIG. 31 is a bar graph showing quantitation of cells from FIG. 30 with rereplication. Data represent the average of three independent experiments±S.D. (−S.D. error bars not depicted). p-values were calculated using Student's t-test. **p<0.01.

FIG. 32 is a series of Western blots of representative individual clones of DM93 cells with hypomorphic expression of p21 (clone sg-p21-1) or SET8 protein (clone sg-SET8-1) treated with pevonedistat for 48 hours (+) or without pevonedistat treatment (−). See also FIGS. 33-38.

FIG. 33 and FIG. 34 present the results of surveyor assays demonstrating the efficient targeting of the CDKN1A (encoding p21; FIG. 33) and SET8 (FIG. 34) genes in the selected individual clones of DM93 melanoma cells (−1 through −6 for each gene correspond to individual clones). DNA extracted from control DM93 clone (sg-control) serves as a negative control. Solid arrows: primer-specific amplification of CDKN1A and SET8 DNA flanking the sg-RNAs (SEQ ID Nos: 17 and 18, respectively) targeted sites. Dashed arrows: cleavage products of the CDKN1A and SET8 DNA following cleavage by the Surveyor nuclease.

FIG. 35 and FIG. 36 show immunoblots of cell lysates extracted from the indicated DM93 or individual clones of DM93 with sg-control, sg-p21 (−2 through −6; FIG. 35) or sg-SET8 (−2 through −6; FIG. 36) and treated with 1 μM pevonedistat for 48 hours. Tubulin was the loading control. Asterisk: cross-reactive band. sg-p21-1 and sg-SET8-1 clones are shown in FIG. 32.

FIG. 37 and FIG. 38 are graphs showing the extent of rereplication in control DM93 cells (sg-control; transfected with a pX330 vector containing a human codon-optimized SpCas9 endonuclease; Catalogue No. 42230, Addgene, Cambridge, Mass., United States of America) but without an sg-RNA or in individual DM93 clones with sg-RNAs targeting CDKN1A (FIG. 37; SEQ ID NO: 18) or SET8 (FIG. 38; SEQ ID NO: 17) and following pevonedistat treatment for 48 hours (1 μM) as determined by propidium iodide (PI) staining and FACS analysis. Data represent the average of three independent experiments±S.D. (−S.D. error bars not depicted). p values were calculated using Student's −t test. *p<0.05; **p<0.01; ***p<0.001.

FIGS. 39 and 40 are bar graphs showing the extent of rereplication (FIG. 39) and senescence (FIG. 40) in the cells shown in FIG. 32. Data represent the average of three independent experiments±S.D. (−S.D. error bars not depicted). p-values were calculated using Student's t-test. *p<0.05; **p<0.01; ***p<0.001.

FIG. 41 is a bar graph, the same as in FIG. 27, but with the sg-control-1, sg-p21-1, and sg-SET8-1 DM93 cells shown in FIG. 32.

FIG. 42 is an immunoblot of DM93 tumor xenografts extracted on day 25, demonstrating inhibition of cullin neddylation, the stabilization of various cullin substrates, and the induction of DNA damage (p53 accumulation and increase in γH2AX) following in vivo pevonedistat administration. Tubulin was a loading control.

FIG. 43 is the result of Western blot analysis of DM331 (R1) and SK-MEL24 (R1) cell extracts following treatment with 1 μN pevonedistat for the indicated times in vitro. Tubulin was a loading control.

FIG. 44 is a bar graph showing the extent of rereplication in DM331 (R1-R3) and SK-MEL-24 (R1 and R2) following treatment with 1 μM pevonedistat for 72 hours (black bars). Vehicle (DMSO) controls are shown as the white bars. Data represent the average of three independent experiments±S.D. (−S.D. error bars not depicted).

FIG. 45 is a Western blot of of protein lysates extracted from control (si-GL2; SEQ ID NO: 1) or CDT2-depleted (si-CDT2; SEQ ID NO: 2) Cal27 or FaDu cells. Actin is shown for loading control.

FIG. 46 is a series of Western blots of protein lysates extracted from Cal27 or FaDu cells treated with the indicated doses of pevonedistat (concentrations in nM listed below the time points) for 24 or 48 hours. Actin is a loading control.

FIGS. 47 and 48 are graphs summarizing the results of in vivo experiments showing that pevonedistat dose-dependently increased radiosensitivity of Cal27 (FIG. 47) and FaDu (FIG. 48) cells. The indicated doses of pevonedistat were administered 24 hours prior to irradiation with the indicated doses. Surviving fractions were determined by dividing the number of colonies present in the cells treated with a particular dose of IR by the number of colonies formed from non-irradiated cells in that treatment group. Data represent the average from three independent experiments±S.D.

FIG. 49 is a graph showing that pevonedistat suppressed HNSCC xenograft growth and further inhibited growth when combined with IR (see details in Materials and Methods for EXAMPLES 8-13). Mean tumor volumes±s.e.m are shown. n=8 mice per group. p values were calculated using Student's f-test; *p<0.05; **p<0.01. Solid circles: DMSO (negative) control; Solid squares: pevonedistat treatment alone; Open circles: irradiation (IR) alone; open squares: combined treatment with pevonedistat and IR.

### BRIEF DESCRIPTION OF THE SEQUENCE LISTING

SEQ ID NO: 1 is the nucleotide sequence of the sense strand of a control siRNA.

SEQ ID NO: 2 is the nucleotide sequence of the sense strand of an siRNA targeted against a CDT2 gene product.

SEQ ID NO: 3 is the nucleotide sequence of the sense strand of an siRNA targeted against a CDT1 gene product.

SEQ ID NO: 4 is the nucleotide sequence of the sense strand of an siRNA targeted against a SET8 gene product.

SEQ ID NO: 5 is the nucleotide sequence of the sense strand of an siRNA targeted against a p21 gene product.

SEQ ID NO: 6 is the nucleotide sequence of the sense strand of an siRNA targeted against a geminin gene product.

SEQ ID NOs: 7 and 8 are the nucleotide sequences of the sense strands of two different siRNAs targeted against an Emil gene product, si-EMI1-1 and si-EMI-2, respectively.

SEQ ID NOs: 9 and 10 are the nucleotide sequences of oligonucleotide primers that can be used together to amplify a subsequence of a CDT2 gene product.

SEQ ID NOs: 11 and 12 are the nucleotide sequences of oligonucleotide primers that can be used together to amplify a subsequence of a SET8 gene product.

SEQ ID NOs: 13 and 14 are the nucleotide sequences of oligonucleotide primers that can be used together to amplify a subsequence of a p21 gene product.

SEQ ID NOs: 15 and 16 are the nucleotide sequences of two single-guide RNAs targeted against a CDT2 gene product.

SEQ ID NO: 17 is the nucleotide sequence of a single-guide RNAs targeted against a SET8 gene product.

SEQ ID NO: 18 is the nucleotide sequence of a single-guide RNAs targeted against a CDKN1A gene product.

### DETAILED DESCRIPTION

Headings are included herein for reference and to aid in locating certain sections. These headings are not intended to limit the scope of the concepts described therein under, and these concepts can have applicability in other sections throughout the entire specification.

### I. GENERAL CONSIDERATIONS

The presently disclosed subject matter relates to compositions and methods for treating cancer, particularly melanoma, including the unexpected result of a synergism between pevonedistat and BRAF kinase inhibitors (e.g., vemurafenib). CDT2 is a substrate receptor for the cullin 4 based CRL4^{CDT2} E3 ubiquitin ligase, an important enzymatic complex, which regulates cell cycle progression primarily through the ubiquitylation and degradation of the replication factor CDT1, the cyclin dependent kinase (CDK) inhibitor p21 and the histone methyltransferase SET8. Knockdown of CDT2 by siRNA or CRISPR/Cas9-mediated deletion of the CDT2 gene inhibited the proliferation of a panel of melanoma cancer cell lines with various genetic backgrounds in vitro and in virus-free head and neck cancer cells. Growth inhibition was associated with SETS- and p21-dependent DNA rereplication and senescence. The presently disclosed subject matter therefore provides for the use of such techniques as siRNA and other gene technologies in treating subjects in need thereof.

Additional studies herein demonstrate that pevonedistat (MLN4924), an inhibitor of protein neddylation necessary for the activity of all cullin-based E3 ligases including CRL4^{CDT2}, is sufficient to halt melanoma proliferation permanently through the induction of robust rereplication and senescence, which correlate with CDT2 expression and are dependent on the stabilization of SET8 and p21 proteins. In vivo studies disclosed herein demonstrate that pevonedistat is effective at inhibiting melanoma xenografts in nude mice through CRL4^{CDT2} inhibition, the stabilization of SET8 and p21 proteins and the induction of rereplication, irrespective of the expression of oncogenic BRAF/NRAS proteins. Importantly, it was determined that when combined with BRAF kinase inhibitors (e.g. vemurafenib), pevonedistat treatment yields synergistic suppression in BRAF mutant melanoma xenograft in mice. In addition, pevonedistat is effective at suppressing vemurafenib-resistant melanoma cells and tumors, demonstrating the potential use of this promising drug as a second-line therapy for patients with relapsed melanomas following BRAF-kinase inhibitor and potentially other melanoma therapeutics.

In non-virus associated head and neck cancer cells (human papilloma virus negative cells (HPV-ve) and tumors pevonedistat was effective as a single monotherapy in suppressing cells and tumors. Importantly, it significantly and synergistically suppressed HPV-ve tumors receiving radiotherapy. Collectively, these results identify pevonedistat as a synergistic agent for BRAF kinase inhibitors for BRAF melanoma and for radiation treatment of head and neck cancer, particularly those that are associated with viral infection.

### II. DEFINITIONS

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter. Thus, unless defined otherwise, all technical and scientific terms and any acronyms used herein have the same meanings as commonly understood by one of ordinary skill in the art in the field of the presently disclosed subject matter. Although any compositions, methods, kits, and means for communicating information similar or equivalent to those described herein can be used to practice the presently disclosed subject matter, particular compositions, methods, kits, and means for communicating information are described herein. It is understood that the particular compositions, methods, kits, and means for communicating information described herein are exemplary only and the presently disclosed subject matter is not intended to be limited to just those embodiments.

The articles “a”, “an”, and “the” are used herein to refer to one or to more than one (i.e., to at least one) of the grammatical object of the article. By way of example, “an element” means one element or more than one element.

As used herein, the term “about” means approximately, in the region of, roughly, or around. When the term “about” is used in conjunction with a numerical range, it modifies that range by extending the boundaries above and below the numerical values set forth. For example, in some embodiments, the term “about” is used herein to modify a numerical value above and below the stated value by a variance of in some embodiments±20%, in some embodiments ±15%, in some embodiments ±10%, in some embodiments ±5%, in some embodiments 1%, in some embodiments 0.5%, in some embodiments ±0.1%, and in some embodiments less than ±0.1%. When the term “about” is used in conjunction with a numerical range, it modifies that range by extending the boundaries above and below the numerical values set forth. In general, the term “about” is used herein to modify a numerical value above and below the stated value by a variance of in some embodiments ±20%, in some embodiments ±15%, in some embodiments ±10%, in some embodiments ±5%, and in some embodiments ±1%, and can include no variance at all or 1%, 2%, 3%, 4%, 5%, 6%, 7%, 8%, 9%, 10%, 11%, 12%, 13%, 14%, 15%, 16%, 17%, 18%, 19%, or 20%. Therefore, about 50% means in the range of in some embodiments 40%-60%, in some embodiments 45%-55%, etc. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

As used herein, the phrases “additional therapeutically active compound”, “additional therapeutic agent”, and the like refer to the use or administration of a compound for an additional therapeutic use for a particular injury, disease, or disorder being treated. Such a compound, for example, could include one being used to treat an unrelated disease or disorder, or a disease or disorder which might not be responsive to the primary treatment for the injury, disease or disorder being treated.

As used herein, the term “adjuvant” refers to a substance that elicits an enhanced immune response when used in combination with a specific antigen.

As use herein, the terms “administration”, “administering”, and grammatical variants thereof in the context of a compound or a composition refer to providing a compound or composition of the presently disclosed subject matter or a prodrug thereof to a subject in need of treatment with the compound, prodrug, or composition.

As used herein, the term “aerosol” refers to suspension in the air. In particular, aerosol refers to the particlization or atomization of a formulation of the presently disclosed subject matter and its suspension in the air.

As used herein, an “agonist” is a composition of matter which, when administered to a mammal such as a human, enhances or extends a biological activity attributable to the level or presence of a target compound or molecule of interest in the mammal.

A disease or disorder is “alleviated” if the severity of a symptom of the disease, condition, or disorder, or the frequency with which such a symptom is experienced by a subject, or both, are reduced or eliminated.

As used herein, amino acids are represented by the full name thereof, by the three-letter code corresponding thereto, and/or by the one-letter code corresponding thereto, as indicated in Table 1:

TABLE 1
Amino Acids and Their Abbreviations

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage can be present or absent in the peptides of the presently disclosed subject matter.

The term “amino acid” is used interchangeably with “amino acid residue” and can refer to a free amino acid and to an amino acid residue of a peptide. It will be apparent from the context in which the term is used whether it refers to a free amino acid or a residue of a peptide.

Amino acids have the following general structure:

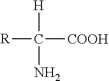

Amino acids can be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as exchanges within one of the following five groups:

(1) Small aliphatic, nonpolar or slightly polar residues: Ala, Ser, Thr, Pro, Gly;

(2) Polar, negatively charged residues and their amides: Asp, Asn, Glu, Gln;

(3) Polar, positively charged residues: His, Arg, Lys;

(4) Large, aliphatic, nonpolar residues: Met, Leu, Ile, Val, Cys; and

(5) Large, aromatic residues: Phe, Tyr, Trp

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

As used herein, an “analog” of a chemical compound is a compound that, by way of example, resembles another in structure but is not necessarily an isomer (e.g., 5-fluorouracil is an analog of thymine).

The term “antagomir” refers to a small RNA or DNA (or chimeric) molecule to antagonize endogenous small RNA regulators like microRNA (miRNA). These antagonists bear complementary nucleotide sequences for the most part, which means that antagomirs should hybridize to the mature microRNA (miRNA). They prevent other molecules from binding to a desired site on an mRNA molecule and are used to silence endogenous microRNA (miR). Antagomirs are therefore designed to block biological activity of these post-transcriptional molecular switches. Like the exemplary target ligands (microRNA, miRNA), antagomirs have to cross membranes to enter a cell. Antagomirs also known as anti-miRs or blockmirs.

An “antagonist” is a composition of matter which when administered to a subject such as a human, inhibits a biological activity attributable to the level or presence of a compound or molecule of interest in the mammal.

As used herein, the term “antibody” refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies of the presently disclosed subject matter can exist in a variety of forms including, for example, polyclonal antibodies, monoclonal antibodies, Fv, Fab and F(ab)₂, as well as single chain antibodies and humanized antibodies (Bird et al., 1988; Houston et al., 1988; Harlow et al., 1989; Harlow et al., 1999).

As used herein, the term “antibody heavy chain” refers to the larger of the two types of polypeptide chains present in all antibody molecules.

As used herein, the term “antibody light chain” refers to the smaller of the two types of polypeptide chains present in all antibody molecules.

As used herein, the term “synthetic antibody” refers to an antibody that is generated using recombinant DNA technology, such as, for example, an antibody expressed by a bacteriophage as described herein. The term should also be construed to refer to an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antimicrobial agents” as used herein refers to any naturally-occurring, synthetic, or semi-synthetic compound or composition or mixture thereof, which is safe for human or animal use as practiced in the methods of this presently disclosed subject matter, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence can be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

As used herein, the term “apoptosis” refers to “programmed cell death” that either naturally occurs or than can be induced in a cell by external stimuli. It is typically characterized by the fragmentation of nuclear DNA (see Taylor et al., 2008)

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “aqueous solution” as used herein can include other ingredients commonly used, such as sodium bicarbonate described herein, and further includes any acid or base solution used to adjust the pH of the aqueous solution while solubilizing a peptide.

The term “basic” or “positively charged” amino acid, as used herein, refers to amino acids in which the R groups have a net positive charge at pH 7.0, and include, but are not limited to, the standard amino acids lysine, arginine, and histidine.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner” as used herein, refers to a molecule capable of binding to another molecule. In some embodiments, a binding partner is a ligand.

The term “biocompatible”, as used herein, refers to a material that does not elicit a substantial detrimental response in the host.

As used herein, the term “biologically active fragments” or “bioactive fragment” of the peptides encompasses natural or synthetic portions of a longer peptide or protein that are capable of specific binding to their natural ligand or of performing the desired function of the protein, for example, a fragment of a protein of larger peptide which still contains the epitope of interest and is immunogenic.

The term “biological sample” as used herein, refers to samples obtained from a subject, including, but not limited to, skin, hair, tissue, blood, plasma, cells, sweat and urine.

A “biomarker” or “marker” is a specific biochemical in the body which has a particular molecular feature that makes it useful for measuring the progress of disease or the effects of treatment, or for measuring a process of interest.

As used herein, the phrase “BRAF inhibitor” refers to a molecule, compound, or composition that inhibits at least one biological activity of a BRAF (also referred to as B-raf or Braf) polypeptide, optionally a human BRAF polypeptide. The BRAF protein is a serine/threonine protein kinase that is involved in signal transduction via the RAS/MAPK pathway. Exemplary BRAF gene products include those described in the GENBANK® biosequence database under the following Accession Numbers: Homo sapiens (NM_004333.4 and NP_004324.2), Gorilla gorilla gorilla (XM_004046322.2 and XP_004046370.1), Pan troglodytes (XM_003951159.3 and XP_003951208.1), Pan paniscus (XM_008965952.1 and XP_008964200.1), Macaca mulatta (XM_015135078.1 and XP_014990564.1), Equus caballus (XM_001496264.5 and XP_001496314.3), Sus scrofa (XM_005654267.2 and XP_005654324.1), Felis catus (XM_011280567.2 and XP_011278869.1), Canis lupus familiaris (XM_014119889.1 and XP_013975364.1), Mus musculus (XM_011241134.2 and XP_011239436.1), and Rattus norvegicus (XM_017602780.1 and XP_017458269.1).

As used herein, the term “cancer” refers to proliferation of cells whose unique trait—loss of normal controls—results in unregulated growth, lack of differentiation, local tissue invasion, and metastasis. Examples include but are not limited to, melanoma, breast cancer, prostate cancer, ovarian cancer, uterine cancer, cervical cancer, skin cancer, pancreatic cancer, colorectal cancer, renal cancer, and lung cancer. The term “tumor” is somewhat broader than but overlaps to some degree with the term “cancer”, a difference being that the latter term is typically reserved for malignant and metastatic types of tumors.

As used herein, the term “carrier molecule” refers to any molecule that is chemically conjugated to the antigen of interest that enables an immune response resulting in antibodies specific to the native antigen.

As used herein, the term “chemically conjugated” or “conjugating chemically” refers to linking the antigen to the carrier molecule. This linking can occur on the genetic level using recombinant technology, wherein a hybrid protein can be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds can also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

As used herein, the term “chemotherapy” refers to the administration of one or more anti-cancer drugs such as but not limited to, antineoplastic chemotherapeutic agents, chemopreventative agents, and/or other agents to a tumor and/or a cancer patient by various methods, including but not limited to intravenous, oral, intramuscular, intraperitoneal, intravesical, subcutaneous, transdermal, buccal, or inhalation or in the form of a suppository. Chemotherapy can be given prior to surgery to shrink a large tumor prior to a surgical procedure to remove it after surgery or radiation therapy to prevent the growth of any remaining cancer cells in the body.

As used herein, the abbreviation “CHX” refers to cyclohexamide (4-[(2R)-2-[(1S,3S,5S)-3,5-Dimethyl-2-oxocyclohexyl]-2-hydroxyethyl]piperidine-2,6-dione; CAS Registry No. 66-81-9).

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound” as used herein, refers to a polypeptide, an isolated nucleic acid, or other agent used in the method of the presently disclosed subject matter.

A “computer-readable medium” is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information can be transferred between a system of interest and a medium, between computers, or between computers and the computer-readable medium for storage or access of stored information. Such transmission can be electrical, or by other available methods, such as IR links, wireless connections, etc.

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a disease or disorder for which the test is being performed.

A “test” cell is a cell being examined, which is in some embodiments compared to a control cell.

A “pathoindicative” cell is a cell which, when present in a tissue, is an indication that the animal in which the tissue is located (or from which the tissue was obtained) is afflicted with a disease or disorder.

A “pathogenic” cell is a cell which, when present in a tissue, causes or contributes to a disease or disorder in the animal in which the tissue is located (or from which the tissue was obtained).

A tissue “normally comprises” a cell if one or more of the cell are present in the tissue in an animal not afflicted with a disease or disorder.

As used herein, “CRL4” refers to a cullin 4 RING E3 ubiquitin ligase gene or gene product. CRL4 is also referred to as interleukin 17 receptor B (IL17RB). Exemplary CRL4/IL17RB gene products include those described in the GENBANK® biosequence database under the following Accession Numbers: Homo sapiens (NM_018725.3 and NP_061195.2), Gorilla gorilla gorilla (XM_004034326.2 and XP_004034374.1), Pan troglodytes XM_001173276.5 and XP_001173276.1), Pan paniscus (XM_003818727.3 and XP_003818775.1), Aacaca mulatta XM_001082504.3 and XP_001082504.2), Equus caballus (XM_005600546.2 and XP_005600603.2), Sus scrofa (XM_005669645.2 and XP_005669702.1), Felis catus XM_006928869.2 and XP_006928931.1), and Canis lupus familiaris (XM_014121946.1 and XP_013977421.1).

As used herein, the term “dabrafenib” refers to N-[3-[5-(2-aminopyrimidin-4-yl)-2-tert-butyl-1,3-thiazol-4-yl]-2-fluorophenyl]-2,6-difluorobenzenesulfonamide, which corresponds to CAS Registry No. 1195765-45-7. Dabrafenib has the following structure:

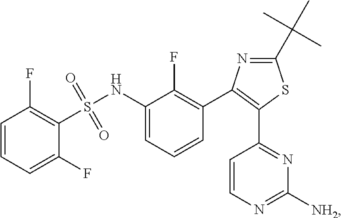 [SMILES: CC(C)(C)c1nc(-c2cccc(NS(=O)(=O)c3c(F)cccc3F)c2F)c(-c2ccnc(N)n2)s1]

and is an inhibitor of BRAF biological activity.

As used herein, the term “delivery vehicle” refers to a composition that facilitates delivery of molecules (in some embodiments, therapeutic molecules or therapeutic agents) to a target. In some embodiments, a delivery vehicle is selected from the group consisting of a liposome, a micelle, an ethosome, a carbon nanotube, a fullerene nanoparticle, a metal nanoparticle, a semiconductor nanoparticle, a polymer nanoparticle, an oxide nanoparticle, a nanoworm, a viral particle, a polyionic particle, and a ceramic particle. In some embodiments, the delivery vehicle is designed to protect the molecule from degradation in an environment (in some embodiments, an environment within or otherwise associated with a subject to which the molecule is to be delivered). In some embodiments, the delivery vehicle is biodegradable.

As used herein, a “derivative” of a compound refers to a chemical compound that can be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, in some embodiments, the term “diagnosis” refers to detecting aberrant expression due to cancers overexpressing CDT2. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the phrase “disease, disorder, or condition associated with CRL4^{CDT2} ubiquitin ligase biological activity” refers to a disease, disorder, or condition at least one symptom or consequence of which results directly or indirectly from a CRL4^{CDT2} ubiquitin ligase biological activity. In some embodiments, a disease, disorder, or condition associated with CRL4^{CDT2} ubiquitin ligase biological activity relates to cellular proliferation (optionally undesirable cellular proliferation), including but not limited to cancer.

As used herein, the phrase “disease, disorder, or condition associated with undesirable cullin signaling” refers to a disease, disorder, or condition at least one symptom or consequence of which results directly or indirectly from signal transduction through the cullin signaling pathway. In some embodiments, a disease, disorder, or condition associated with undesirable cullin signaling relates to cellular proliferation (optionally undesirable cellular proliferation), including but not limited to cancer.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), can be different from when that compound is administered alone. Thus, an effective amount of a combination of compounds refers collectively to the combination as a whole, although the actual amounts of each compound can vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

A “fragment” or “segment” is a portion of an amino acid sequence, comprising at least one amino acid, or a portion of a nucleic acid sequence comprising at least one nucleotide.

The terms “fragment”, “subsequence”, and “segment” are used interchangeably herein.

As used herein, the term “fragment” as applied to a protein or peptide, can be in some embodiments at least about 3-15 amino acids in length, in some embodiments at least about 15-25 amino acids, in some embodiments at least about 25-50 amino acids in length, in some embodiments at least about 50-75 amino acids in length, in some embodiments at least about 75-100 amino acids in length, and in some embodiments greater than 100 amino acids in length.

As used herein, the term “fragment” as applied to a nucleic acid, can be in some embodiments at least about 20 nucleotides in length, typically, in some embodiments at least about 50 nucleotides, in some embodiments from about 50 to about 100 nucleotides, in some embodiments at least about 100 to about 200 nucleotides, in some embodiments at least about 200 nucleotides to about 300 nucleotides, in some embodiments at least about 300 to about 350, in some embodiments at least about 350 nucleotides to about 500 nucleotides, in some embodiments at least about 500 to about 600, in some embodiments at least about 600 nucleotides to about 620 nucleotides, in some embodiments at least about 620 to about 650, and in some embodiments the nucleic acid fragment can be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 500% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993. This algorithm is incorporated into the NBLAST and XBLAST programs of Altschul, et al., 1990, and can be accessed, for example, via the world wide web site of the United States National Library of Medicine's National Center for Biotechnology Information (NCBI). BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastx” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST), can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit” as used herein when referring to a function, refers to the ability of a compound of the presently disclosed subject matter to reduce or impede a described function. Inhibition can be in some embodiments by at least 10%, in some embodiments by at least 25%, in some embodiments by at least 50%, and in some embodiments the function is inhibited by at least 75%. When the term “inhibit” is used more generally, such as “inhibit Factor I”, it refers to inhibiting expression, levels, and activity of Factor I.

The term “inhibit a complex” as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

In some embodiments, an inhibitor is an inhibitory nucleic acid, optionally an inhibitory RNA. As used herein, the phrase “inhibitory nucleic acid” refers to a ribonucleic acid molecule that can be employed to inhibit a biological activity of a target gene product. Exemplary inhibitor nucleic acids include, but are not limited to double-stranded RNAs (dsRNAs; see e.g., U.S. Patent Application Publication No. 2015/0047064), siRNAs (see e.g., U.S. Pat. Nos. 8,148,345 and 8,383,599), and single-guide RNAs (see e.g., U.S. Pat. No. 8,697,359). In some embodiments, an inhibitory nucleic acid is an anti-CRL4^{CDT2} ubiquitin ligase inhibitory nucleic acid, an anti-p21 inhibitory nucleic acid, an anti-CDT1 inhibitory nucleic acid, an anti-SET8 inhibitory nucleic acid, an anti-geminin inhibitory nucleic acid, an anti-CDKN1A inhibitory nucleic acid, an anti-EMI1 inhibitory nucleic acid, or any combination thereof.

The term “inhibit a protein” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting”, “applying”, and “administering” includes administration of a compound of the presently disclosed subject matter by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material can describe one or more methods of alleviating the diseases or disorders in a cell or a tissue of a mammal. The instructional material of the kit of the presently disclosed subject matter can, for example, be affixed to a container which contains the identified compound presently disclosed subject matter or be shipped together with a container which contains the identified compound. Alternatively, the instructional material can be shipped separately from the container with the intention that the instructional material and the compound be used cooperatively by the recipient.

The term “isolated” when used in reference to cells, refers to a single cell of interest, or population of cells of interest, at least partially isolated from other cell types or other cellular material with which it naturally occurs in the tissue of origin (e.g., adipose tissue). A sample of stem cells is “substantially pure” when it is at least 60%, or at least 75%, or at least 90%, and, in certain cases, at least 99% free of cells other than cells of interest. Purity can be measured by any appropriate method, for example, by fluorescence-activated cell sorting (FACS), or other assays which distinguish cell types.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression can cause or contribute to the disease or disorder, be a symptom of the disease or disorder, or both.

As used herein, “MAPK” refers to a family of mitogen-activated protein kinase genes and gene products. It also refers in general to cellular signaling pathways that involve members of the MAPK family (see e.g., Cargnello & Roux, 2011; Plotnikov et al., 2011).

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

As used herein, the phrase “MEK inhibitor” refers to refers to a molecule, compound, or composition that inhibits at least one biological activity of a dual specificity mitogen-activated protein kinase kinase 1 (MAP2K1/MEK1) or dual specificity mitogen-activated protein kinase kinase 2 (MAP2K2/MEK2) polypeptide, optionally a human MAP2K1/MEK1 or MAP2K2/MEK2 polypeptide. The MAP2K1/MEK1 and MAP2K2/MEK2 proteins are dual specificity protein kinases that are involved in signal transduction via the MAP kinase pathway. Exemplary MAP2K1/MEK1 gene products include those described in the GENBANK® biosequence database under the following Accession Numbers: Homo sapiens (NM_002755.3 and NP_002746.1), Pan troglodytes (NM_001009071.1 and NP_001009071.1), Macaca mulatta (NM_001257549.1 and NP_001244478.1), Equus caballus (XM_001496420.5 and XP_001496470.3), Sus scrofa (NM_001143716.1 and NP_001137188.1), Felis catus (XM_003987018.4 and XP_003987067.1), Canis lupus familiaris (NM_001048094.2 and NP_001041559.2), Mus musculus (NM_008927.3 and NP_032953.1), and Rattus norvegicus (NM_031643.4 and NP_113831.1). Exemplary MAP2K2/MEK2 gene products include those described in the GENBANK® biosequence database under the following Accession Numbers: Homo sapiens (NM_030662.3 and NP_109587.1), Pan troglodytes (NM_001009071.1 and NP_001009071.1), Macaca mulatta (XM_015122493.1 and XP_014977979.1), Equus caballus (XM_014741063.1 and XP_014596549.1), Sus scrofa (NM_001244550.1 and NP_001231479.1), Canis lupus familiaris (NM_001048136.1 and NP_001041601.1), Mus musculus (NM_023138.5 and NP_075627.2), and Ratus norvegicus (NM_133283.1 and NP_579817.1).

As used herein, “NEDD8” refers to a neural precursor cell expressed, developmentally down-regulated 8 gene and gene product. Exemplary NEDD8 gene products include those described in the GENBANK® biosequence database under the following Accession Numbers: Homo sapiens (NM_006156.2 and NP_006147.1), Mus musculus (NM_008683.3 and NP_032709.1), Gorilla gorilla gorilla (XM_004055019.2 and XP_004055067.1), Pan troglodytes (NM_016925926.1 and XP_016781415.1), Macaca mulatta (XM_015146891.1 and XP_015002377.1), and Gallus gallus (XM_015273745.1 and XP_015129231.1).

The term “NEDD8-activating enzyme” (NAE) refers to a dimeric enzyme that comprises catalytic and regulatory subunits (see U.S. Pat. No. 6,734,283; see also Gong & Yeh, 1999; Read et al., 2000; Chiba & Tanaka, 2004; Petroski & Deshaies, 2005). In humans, the catalytic subunit (e.g., GENBANK® Accession No. NP_003959.3) is encoded by the NEDD8-activating enzyme E1 catalytic subunit gene (e.g., GENBANK® Accession No. NM_003968.3). The regulatory subunit (e.g., GENBANK® Accession No. NP_003896.1) is encoded by the NEDD8-activating enzyme E1 regulatory subunit gene (e.g., GENBANK® Accession No. NM_003905.3).

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid” “DNA” “RNA” and similar terms also include nucleic acid analogs, i.e. analogs having other than a phosphodiester backbone. For example, the so-called “peptide nucleic acids” which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Operably linked” refers to a juxtaposition wherein the components are configured so as to perform their usual function. Thus, control sequences or promoters operably linked to a coding sequence are capable of effecting the expression of the coding sequence. By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, the phrase “overcoming vemurafenib-resistance in a cell” refers to any treatment that renders a cell that had previously developed resistance to vemurafenib more susceptible to killing as a result of said treatment than the cell would have been in the absence of the treatment. Mechanisms that can give rise to resistance to vemurafenib have been described, including the acquisition of NRAS-activiting somatic mutations (see Romano et al., 2013) In some embodiments, “overcoming vemurafenib-resistance in a cell” comprises treating the cell with a different therapeutic agent, which in some embodiments can be pevonedistat.

As used herein, the term “overexpress” and grammatical variants thereof refers to refers to a level at which a gene product is expressed in one cell as compared to another similar cell. While the term can be applied to wild-type (i.e., normal) cells of different types or at different times and/or stages of development, it is typically employed to compare gene expression levels of cancer and/or tumor cells vis-à-vis their normal (i.e., non-cancerous and/or non-tumorigenic) counterparts. Thus, as described herein, CDT2 is overexpressed in cutaneous melanoma, which means that the expression level of CDT2 is higher in cells of cutaneous melanoma as compared to normal cutaneous cells, but also means that for a given cell, the expression level of CDT2 is higher in the cutaneous melanoma cell that it was prior to the cell becoming a cutaneous melanoma cell.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides, and in some embodiments refers to subsequences of longer polypeptides. Peptide lengths can be in some embodiments 100 amino acids or fewer, in some embodiments 50 amino acids or fewer, in some embodiments 30 amino acids or fewer, in some embodiments 25 amino acids or fewer, in some embodiments 20 amino acids or fewer, in some embodiments 15 amino acids or fewer, and in some embodiments 10 amino acids or fewer. It is noted that with respect to these specific size ranges, each and every whole number between 1 and 100 inclusive is explicitly disclosed (i.e., 1, 2, 3, 4, 5, 6, 7, 8, 9, 10, 11, 12, 13, 14, 15, 16, 17, 18, 19, 20, 21, 22, 23, 24, 25 . . . 100).

The term “per application” as used herein refers to administration of a drug or compound to a subject.

As used herein, the term “pevonedistat” refers to a compound of the following formula that is a small molecule inhibitor of Nedd8-activating enzyme biological activity.

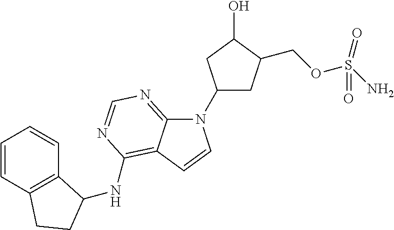 [SMILES: NS(=O)(=O)OCC1CC(n2ccc3c(NC4CCc5ccccc54)ncnc32)CC1O]

Its IUPAC/Chemical name is ((1S,2S,4R)-4-(4-((1S)-2,3-Dihydro-H-inden-1-ylamino)-7H-pyrrolo(2,3-d)pyrimidin-7-yl)-2-hydroxycyclopentyl)methyl sulphamate, and it is also referred to as MLN-4924. Its Chemical Abstract Service (CAS) No. is 905579-51-3. See also Czuczman et al., 2015.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan. As such, “pharmaceutical compositions” include formulations for human and veterinary use.

“Pharmaceutically acceptable” means physiologically tolerable in the relevant subject or subject population. Thus, the phrase “pharmaceutically acceptable carrier” means a chemical composition with which an appropriate compound or derivative can be combined and which, following the combination, can be used to administer the appropriate compound to a subject. In some embodiments, “pharmaceutically acceptable” refers to acceptable for use in humans and/or mammals, and/or in veterinary applications. In some embodiments, a pharmaceutically acceptable composition is pharmaceutically acceptable for use in a human, meaning that the composition would be medically appropriate for use in a human for one or more purposes. In particular, the phrase “pharmaceutically acceptable for use in a human” means that the composition is in some embodiments generally recognized as being safe (GRAS) for human consumption and/or administration.

As used herein, the term “physiologically acceptable” ester or salt means an ester or salt form of the active ingredient which is compatible with any other ingredients of the pharmaceutical composition, which is not deleterious to the subject to which the composition is to be administered.

As used herein, “PI3K” refers to a phosphatidyl-inositol 3-kinase gene and gene product, which are responsible for the production of phosphatidylinositol 3-phosphate, phosphatidylinositol (3,4)-bisphosphate, and phosphatidylinositol (3,4,5)-trisphosphate. PI3Ks are heterodimeric proteins that have regulatory and catalytic subunits. Exemplary PI3K gene products include, but are not limited to GENBANK® Accession Nos. NP_006210.1 (PI3K catalytic subunit beta isoform isoform 1, encoded by GENBANK® Accession No. NM_006219.2) and NP_852664.1 (phosphatidylinositol 3-kinase regulatory subunit alpha isoform 1; encoded by GENBANK® Accession No. NM_181523.2).

“Plurality” means at least two. A plurality can be in some embodiments 2 or 3, in some embodiments between 2 and 4 inclusive, in some embodiments between 2 and 5 inclusive, in some embodiments between 2 and 6 inclusive, in some embodiments between 2 and 7 inclusive, in some embodiments between 2 and 8 inclusive, in some embodiments between 2 and 9 inclusive, in some embodiments between 2 and 10 inclusive, and in some embodiments greater than 10.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

A “polynucleotide” means a single strand or parallel and anti-parallel strands of a nucleic acid. Thus, a polynucleotide can be either a single-stranded or a double-stranded nucleic acid.

The term “prevent” as used herein, means to stop something from happening, or taking advance measures against something possible or probable from happening. In the context of medicine, “prevention” generally refers to action taken to decrease the chance of getting a disease, disorder, or condition. It is recognized, however, that particularly in the context of medicine, “prevention” is not to be interpreted absolutely and that the term includes circumstances under which a chance of getting a disease, disorder, or condition is reduced within a population and/or for an individual and/or the time period under which a population and/or an individual acquires and/or develops a disease, disorder, or condition is delayed relative to that time frame under which the population and/or the individual would have acquired and/or developed the disease, disorder, or condition absent the action taken.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but can be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with one or more detectable moieties, such as but not limited to chromogenic, radioactive, and/or fluorescent moieties and used as detectable agents.

As used herein, the term “promoter/regulatory sequence” means a nucleic acid sequence which is required for expression of a gene product operably linked to the promoter/regulator sequence. In some instances, this sequence can be the core promoter sequence and in other instances, this sequence can also include an enhancer sequence and other regulatory elements which are required for expression of the gene product. The promoter/regulatory sequence can, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of developing pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “providing a prognosis” refers to providing information regarding the impact of the presence of cancer (e.g., as determined by the diagnostic methods of the presently disclosed subject matter) on a subject's future health (e.g., expected morbidity or mortality, the likelihood of getting cancer, and the risk of metastasis).

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, “radiation therapy”, “radiotherapy”, and “irradiation” refer to exposing a subject to high-energy radiation, including without limitation x-rays, gamma rays, and neutrons. This type of therapy includes without limitation external-beam therapy, internal radiation therapy, implant radiation, brachytherapy, and systemic radiation therapy. In some embodiments, radiotherapy is employed in a combination therapy with a composition of the presently disclosed subject matter to treat a disease, disorder, or condition.

A “recombinant cell” is a cell that comprises a transgene. Such a cell can be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

“Recombinant polynucleotide” refers to a polynucleotide having sequences that are not naturally joined together. An amplified or assembled recombinant polynucleotide can be included in a suitable vector, and the vector can be used to transform a suitable host cell. A recombinant polynucleotide can serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

As used herein, the term “reporter gene” means a gene, the expression of which can be detected using a known method. By way of example, the Escherichia coli lacZ gene can be used as a reporter gene in a medium because expression of the lacZ gene can be detected using known methods by adding the chromogenic substrate o-nitrophenyl-β-galactoside to the medium (Gerhardt et al., 1994).

As used herein, the term “rereplication” refers to aberrant replication in which a cell's genome is replicated more than once during a given cell cycle. Generally, rereplication occurs as a consequence of cell cycle defects rather than as part of normal cellular activity, and frequently leads to cellular senescence. Rereplication has also been associated with tumorigenesis in humans (see e.g., Truong & Wu, 2011).

A “sample” as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

As used herein, “sg-RNA” refers to a single guide RNA. Sg-RNAs are typically synthetic RNA molecules that comprise a targeting sequence and a scaffold sequence, and are used to target the Cas9 nuclease to a target nucleotide sequence. See Jinek et al., 2012. See also U.S. Pat. Nos. 9,260,752 and 9,410,198.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

As used herein, the phrase “small interfering RNA (siRNA)” refers, inter alia, to an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. See e.g., Ketting et al., 2001; Hutvagner & Zamore, 2002; Martinez et al., 2002; Provost et al., 2002; Tabara et al, 2002. See also U.S. Pat. No. 7,691,997.

As used herein, the term “solid support” relates to a solvent insoluble substrate that is capable of forming linkages (in some embodiments covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

As used herein, the term “sorafenib” refers to 4-[4-[[4-chloro-3-(trifluoromethyl)phenyl]carbamoylamino]phenoxy]-N-methyl-pyridine-2-carboxamide. It corresponds to CAS Registry No. 284461-73-0. Sorafenib has the following structure:

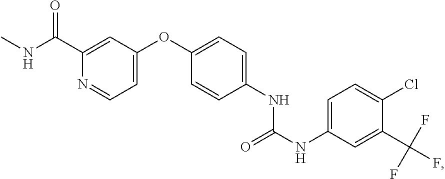 [SMILES: CNC(=O)c1cc(Oc2ccc(NC(=O)Nc3ccc(Cl)c(C(F)(F)F)c3)cc2)ccn1]

and is a multi-kinase inhibitor that inhibitors several tyrosine protein kinases, including BRAF.

By the term “specifically binds to”, as used herein, is meant when a compound or ligand functions in a binding reaction or assay conditions which is determinative of the presence of the compound in a sample of heterogeneous compounds.

The term “standard” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

The term “subject” as used herein refers to a member of any invertebrate or vertebrate species. Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, a “subject in need thereof” is a patient, animal, mammal, or human, who will benefit from the compositions and methods of the presently disclosed subject matter.

As used herein, a “substantially homologous amino acid sequences” includes those amino acid sequences which have in some embodiments at least about 75% homology, in some embodiments at least about 80% homology, in some embodiments at least about 85% homology, in some embodiments at least about 90% homology, in some embodiments at least about 95% homology, in some embodiments at least about 96% homology, in some embodiments at least about 97% homology, in some embodiments at least about 98% homology, and in some embodiments at least about 99% or more homology to an amino acid sequence of a reference antibody chain. Amino acid sequence similarity or identity can be computed by using the BLASTP and TBLASTN programs, which employ a BLAST (basic local alignment search tool) algorithm such as but not limited to the version 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is in some embodiments at least about 50%, in some embodiments at least about 65%, in some embodiments at least about 75%, in some embodiments at least about 85%, in some embodiments at least about 95%, and in some embodiments at least about 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: in some embodiments 7% sodium dodecyl sulfate SDS, 0.5 M NaPO₄, 1 mM EDTA at 50° C. with washing in 2× standard saline citrate (SSC), 0.1% SDS at 50° C.; in some embodiments 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and in some embodiments 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul & Lipman, 1990; Altschul et al., 1990; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. A compound is substantially pure when in some embodiments at least 10%, in some embodiments at least 20%, in some embodiments at least 50%, in some embodiments at least 60%, in some embodiments at least 75%, in some embodiments at least 90%, in some embodiments at least 95%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

As used herein, the term “surgery” refers to any therapeutic and/or diagnostic procedure that involves methodical action of the hand and/or of the hand with an instrument, on the body of a human or other subject, to produce a curative, remedial, and/or diagnostic effect.

The term “symptom” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “trametinib” refers to N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide. It corresponds to CAS Registry No. 871700-17-3 and has the following structure:

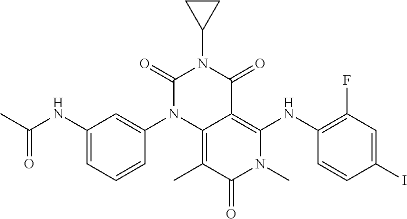 [SMILES: CC(=O)Nc1cccc(-n2c(=O)n(C3CC3)c(=O)c3c(Nc4ccc(I)cc4F)n(C)c(=O)c(C)c32)c1]

Trametinib is an MEK inhibitor, which inhibits both MEK1 and MEK2.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

The term to “treat” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

A “variant”, as described herein, refers to a segment of DNA that differs from the reference DNA. A “marker” or a “polymorphic marker”, as defined herein, is a variant. Alleles that differ from the reference are referred to as “variant” alleles.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

As used herein, the term “vemurafenib” refers to a compound of the following formula that is a small molecule inhibitor of B-Raf biological activity.

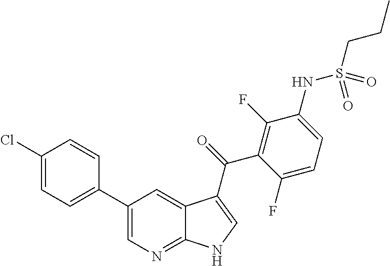 [SMILES: CCCS(=O)(=O)Nc1ccc(F)c(C(=O)c2c[nH]c3ncc(-c4ccc(Cl)cc4)cc23)c1F]

Its IUPAC/Chemical name is N-(3-(5-(4-chlorophenyl)-1H-pyrrolo[2,3-b]pyridine-3-carbonyl)-2,4-difluorophenyl)propane-1-sulfonamide, and it is also referred to as PLX-4032. Its Chemical Abstract Service (CAS) No. is 1029872-54-5.

### III. EMBODIMENTS

In some embodiments, the presently disclosed subject matter provides a composition useful as a therapeutic for treating cancer in a subject in need thereof.

In some embodiments, the presently disclosed subject matter provides compositions and methods for diagnosing cancer.

In some embodiments, a treatment regimen can be developed based on the diagnosis.

In some embodiments, the cancer is selected from the group consisting of melanoma, ovarian cancer, breast cancer, head and neck cancer, lung cancer, carcinosarcoma of the uterus (also known as malignant mixed Mullerian tumor, MMMT), bladder cancer, uterine cancer, endometrial cancer, liver cancer, pancreatic cancer, esophageal cancer, stomach cancer, cervical cancer, prostate cancer, adrenal cancer, lymphoma, leukemia, salivary gland cancer, bone cancer, brain cancer, cerebellar cancer, colon cancer, rectal cancer, colorectal cancer, oronasopharyngeal cancer, nasopharyngeal cancer (NPC), kidney cancer, skin cancer, basal cell carcinoma, hard palate carcinoma, squamous cell carcinoma of the tongue, meningioma, pleomorphic adenoma, astrocytoma, chondrosarcoma, cortical adenoma, hepatocellular carcinoma, pancreatic cancer, squamous cell carcinoma, and adenocarcinoma. In some embodiments, the cancer is melanoma.

In some embodiments, the treatment encompasses a combination therapy.

The methods and compositions of the presently disclosed subject matter encompass multiple regimens and dosages for administering the compounds of the presently disclosed subject matter for use in preventing and treating cancer. For example, a subject can be administered one or more compounds of the presently disclosed subject matter once or more than once. The frequency and number of doses can vary based on many parameters, including the age, sex, and health of the subject. In some embodiments, up to 50 doses are administered. In some embodiments, up to 40 doses are administered, and in some embodiments up to 30 doses are administered. In some embodiments, up to 20 doses are administered, and in some embodiments up to 10 doses are administered. In some embodiments, 5-10 doses are administered. In some embodiments, 5, 6, 7, 8, 9, or 10 doses can be administered.

In some embodiments, the compounds are administered daily, in another weekly, and in another, monthly. Treatment periods can be for a few days, or about a week, or about several weeks, or for several months. Follow-up administration or boosters can be used as well and the timing of that can be varied.

The amount of compound administered per dose can vary as well. For example, in some embodiments the compositions and methods of the presently disclosed subject matter include a range of compound amounts between about 10 micrograms of each or protein per dose to about 10,000 micrograms of protein per dose. In some embodiments, the number of micrograms is the same for each compound. In some embodiments, the number of micrograms is not the same for each compound. In some embodiments, the range of amounts of each compound administered per dose is from about 20 micrograms to about 1,000 micrograms. In some embodiments, it is from about 50 micrograms to about 500 micrograms. In some embodiments, it is from about 75 micrograms to about 400 micrograms. In some embodiments, it is from about 100 micrograms to about 300 micrograms, and in some embodiments it is from about 150 micrograms to about 250 micrograms. In some embodiments, about 300 micrograms of each compound is used per dose per treatment.

Subjects can be monitored before and after administration.

The presently disclosed subject matter encompasses administering the compounds of the presently disclosed subject matter based on the particular cancer being treated, its location in the subject, etc. In some embodiments, a composition is administered by a route selected from the group consisting of intratumoral, parenteral, intravenous, topical, and direct.

Liposomes have certain advantages over the solid core particles previously used, such as the ability to deliver imaging agents or biologically active drugs in their aqueous core or lipid bilayer. Liposomes provide a flexible platform for delivering both hydrophobic and hydrophilic cargo. Liposomes can be useful for both diagnostic imaging and delivery of therapeutic agents to the tumor microenvironment. In some embodiments, the compositions and methods of the presently disclosed subject matter are useful for detecting, identifying, diagnosing, and treating cancer.

In some embodiments, a liposome of the presently disclosed subject matter is about 200 nm in diameter. In some embodiments, a liposome of the presently disclosed subject matter has a diameter ranging from about 100 nm to about 300 nm. In some embodiments, a liposome of the presently disclosed subject matter is about 150 nm in diameter. In some embodiments, a liposome of the presently disclosed subject matter is about 250 nm in diameter.

In some embodiments, a liposome of the presently disclosed subject matter comprises DOTA and optionally at least one other agent or drug. In some embodiments, a liposome of the presently disclosed subject matter is prepared according to the following method with the following components: 18.8 mg/mL of L-a-Phosphatidylcholine, 4.2 mg/mL of cholesterol, and optionally 0.025 mg/mL of the lipophilic fluorescent probe 3,3′-Dioctadecyloxacarbocyanine Perchlorate. A fluorescent probe is added if there will be fluorescent imaging used later. The liposomes are made using dehydration-rehydration: the lipids and DiO are dissolved in chloroform, the solvent is evaporated, and the resultant thin-film hydrated with a 10 mM solution of chelating agent 1,4,7,10-tetra-azacyclododecane-1,4,7,10-tetraacetic acid (DOTA) in 10 mM 4-(2-Hydroxyethyl)-1-Piperazine-Ethanesulfonic Acid (HEPES) buffer with 150 mM NaC and a pH of 4 for 2 hours at 37° C. and overnight at 4° C. The liposome solution is freeze-thawed 5 times and then extruded consecutively 20 times through 1 μm, 600 nm, 400 nm and 200 nm polycarbonate membrane filters using a Lipex extruder with high-pressure nitrogen. The non-encapsulated DOTA is removed by dialysis using a Slide-A-Lyzer G2 dialysis cassette with a molecular weight cut-off of 10,000 against five-2 liters of HEPES buffer containing 150 mM NaCl (pH 7.4).

In some embodiments, a liposome of the presently disclosed subject matter can be labeled for imaging. In some embodiments, the label is a radiolabel. In some embodiments, remote loading is used to radiolabel DOTA-containing liposomes with a useful PET probe, such as ⁶⁴Cu (t½=12.7 h), by utilizing the lipophilic transporter hydroxyquinoline to ferry ⁶⁴Cu to the liposome interior where it is more tightly chelated by the encapsulated DOTA. Copper loading of the liposomes is confirmed using size exclusion chromatography (SEC) column to determine if the fluorescent dye DiO labeled liposomes eluted in the same fractions as the radioactive ⁶⁴Cu. One of ordinary skill in the art will appreciate that the method can be modified as long the result is the same. In some embodiments, the radioactive isotope is selected from the group consisting of ¹¹C, ¹³N, ¹⁵O, ¹⁸F, ⁶⁴Cu, ⁶²Cu, ¹²⁴I, ⁷⁶Br, ⁸²Rb and ⁶⁸Ga. In some embodiments, the chelating agent is selected from the group consisting of DTPA, DO3A, DOTA, EDTA, TETA, EHPG, HBED, NOTA, DOTMA, TETMA, PDTA, TTHA, LICAM, HYNIC, and MECAM.

In some embodiments, a liposome of the presently disclosed subject matter can be labeled with more than one type of imaging agent to allow the liposome, or cells targeted by the liposome, to be imaged or tracked using more than one detection method. For example, both a radiolabel and a fluorescent label can be used at the same time.

The liposomes can be administered to a subject using various techniques. The amount of liposome administered can vary and can depend on the age, sex, and health of the subject, as well as the type of cancer to be imaged. For example, liposomes can be administered at doses from about 0.1 to about 100 μmol total phospholipid. One of ordinary skill in the art can determine a dose to be used. The amount of label can very depending on the label used and the imaging technique used. For example, when using ⁶⁴Cu, the present application discloses that the liposome dose was 1.9 μmol total phospholipid labeled with 50-75 μCi (1.85-2.8 MBq) of ⁶⁴Cu in a total volume of 160 μL. In some embodiments, 100 to 10,000 μCi is used. In some embodiments, 500 to 1,000 μCi is used. In some embodiments, 400-500 μCi is used.

Useful detectable labels, depending on the technique or combination of imaging techniques used, include, but are not limited to, a radionuclide, a radiological contrast agent, a paramagnetic ion, a metal, a biological tag, a fluorescent label, a chemiluminescent label, an ultrasound contrast agent, and a photoactive agent.

Useful radionuclides of the presently disclosed subject matter include, but are not limited to, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ^{52m}Mn, ⁵⁵Co, ⁷²As, ⁷⁵Br, ⁷⁶Br, ⁸²mRb, ⁸³Sr, or other gamma-, beta-, or positron-emitters.

An additional therapeutic agent can include, for example, at least one of a chemotherapeutic agent, an antimicrobial, an anesthetic, an anti-inflammatory, etc.

The peptides of the presently disclosed subject matter can be readily prepared by standard, well-established techniques, such as solid-phase peptide synthesis (SPPS) as described by Bodanszky & Bodanszky, 1984; Stewart et al., 1984. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis can be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, can also be used to determine definitely the sequence of the peptide. Prior to its use, the peptide is purified to remove contaminants. In this regard, it will be appreciated that the peptide will be purified so as to meet the standards set out by the appropriate regulatory agencies. Any one of a number of a conventional purification procedures can be used to attain the required level of purity including, for example, reversed-phase high-pressure liquid chromatography (HPLC) using an alkylated silica column such as C4-, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof can incorporate amino acid residues which are modified without affecting activity. For example, the termini can be derivatized to include blocking groups, i.e. chemical substituents suitable to protect and/or stabilize the N- and C-termini from “undesirable degradation” a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without effect on peptide activity.

Other modifications can also be incorporated without adversely affecting the activity and these include, but are not limited to, substitution of one or more of the amino acids in the natural L-isomeric form with amino acids in the D-isomeric form. Thus, the peptide can include one or more D-amino acid resides, or can comprise amino acids which are all in the D-form. Retro-inverso forms of peptides in accordance with the presently disclosed subject matter are also contemplated, for example, inverted peptides in which all amino acids are substituted with D-amino acid forms.

Acid addition salts of the presently disclosed subject matter are also contemplated as functional equivalents. Thus, a peptide in accordance with the presently disclosed subject matter treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the presently disclosed subject matter.

The presently disclosed subject matter also provides for homologs of proteins and peptides. Homologs can differ from naturally occurring proteins or peptides by conservative amino acid sequence differences or by modifications which do not affect sequence, or by both.

For example, conservative amino acid changes can be made, which although they alter the primary sequence of the protein or peptide, do not normally alter its function. To that end, 10 or more conservative amino acid changes typically have no effect on protein function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Homologs of such polypeptides include those containing residues other than naturally occurring L-amino acids, e.g., D-amino acids or non-naturally occurring synthetic amino acids. The peptides of the presently disclosed subject matter are not limited to products of any of the specific exemplary processes listed herein.

Substantially pure protein or peptide obtained as described herein can be purified by following known procedures for protein purification, wherein an immunological, enzymatic, or other assay is used to monitor purification at each stage in the procedure. Protein purification methods are well known in the art, and are described, for example in Deutscher et al., 1990.

Typical dosage regimens comprise administering a dosage of in some embodiments 1-1000 μg/kg, in some embodiments 10-500 μg/kg, and in some embodiments 10-150 μg/kg, once, twice, or three times a week for a period of one, two, three, four, or five weeks. In some embodiments, 10-100 μg/kg is administered once a week for a period of one or two weeks.

The present method, in some embodiments, comprises administration of the compounds and compositions comprising them via the injection, transdermal, or oral route. In some embodiments of the presently disclosed subject matter, the present method comprises intratumoral administration of the present compounds and compositions comprising them.

In some embodiments, the presently disclosed subject matter relates to pharmaceutical preparations comprising as the active ingredient(s) the present source of a compound as defined herein before. More particular pharmaceutical preparations comprise as the active ingredient(s) one or more of the aforementioned compounds and biologically active analogs thereof.

The presently disclosed subject matter further provides a pharmaceutical preparation comprising one or more of the compounds of the presently disclosed subject matter. The concentration of said compounds in the pharmaceutical composition can vary widely, i.e., from less than about 0.1% by weight, usually being at least about 1% by weight to as much as 20% by weight or more.

The composition can comprise a pharmaceutically acceptable carrier in addition to the active ingredient. The pharmaceutical carrier can be any compatible, non-toxic substance suitable to deliver the compounds to the subject or to a specific site in the subject. For some compounds, sterile water, alcohol, fats, waxes, and inert solids can be used as the carrier. Pharmaceutically acceptable adjuvants, buffering agents, dispersing agents, and the like, can also be incorporated into the pharmaceutical compositions.

A composition for intravenous infusion could be made up to contain 10 to 50 ml of sterile 0.9% NaCl or 5% glucose optionally supplemented with a 20% albumin solution and in some embodiments between 10 μg and 50 mg, and in some embodiments between 50 μg and 10 mg, of the polypeptide. A typical pharmaceutical composition for intramuscular injection would be made up to contain, for example, 1-10 ml of sterile buffered water and in some embodiments between 10 μg and 50 mg and in some embodiments between 50 ug and 10 mg of the polypeptide of the presently disclosed subject matter. Methods for preparing parenterally administrable compositions are well known in the art and described in more detail in various sources, including, for example, in Remington's Pharmaceutical Science (18th ed., Mack Publishing, Easton, Pa., 1990; incorporated by reference in its entirety for all purposes).

### IV. AMINO ACID SUBSTITUTIONS

In certain embodiments, the disclosed methods and compositions can involve preparing peptides with one or more substituted amino acid residues. In various embodiments, the structural, physical and/or therapeutic characteristics of peptide sequences can be optimized by replacing one or more amino acid residues.

In some embodiments, the presently disclosed subject matter encompasses the substitution of a serine or an alanine residue for a cysteine residue in a peptide of the presently disclosed subject matter. Support for this includes what is known in the art. For example, see Kittlesen et al., 1998 for justification of such a serine or alanine substitution.

Other modifications can also be incorporated without adversely affecting the activity and these include, but are not limited to, substitution of one or more of the amino acids in the natural L-isomeric form with amino acids in the D-isomeric form. Thus, the peptide can include one or more D-amino acid resides, or can comprise amino acids which are all in the D-form. Retro-inverso forms of peptides in accordance with the presently disclosed subject matter are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art. For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′,-3′,- or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids can be considered (Kyte & Doolittle, 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−08); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are in some embodiments within +/−2, in some embodiments within +/−1, and in some embodiments within +/−0.5.

Amino acid substitution can also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In some embodiments, replacement of amino acids with others of similar hydrophilicity is employed.

Other considerations include the size of the amino acid side chain. For example, it would generally not be desirable to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see e.g., Chou & Fasman, 1974; Chou & Fasman, 1978; Chou & Fasman, 1979).

Based on such considerations and extensive empirical study, tables of conservative amino acid substitutions have been constructed and are known in the art. For example: arginine and lysine; glutamate and aspartate; serine and threonine; glutamine and asparagine; and valine, leucine, and isoleucine. In some embodiments, a conservative amino acid substitution is selected from the following: for Ala (A): Leu, Ile, or Val; for Arg (R): Gin, Asn, or Lys; for Asn (N): His, Asp, Lys, Arg, or Gin; for Asp (D): Asn or Glu; for Cys (C): Ala or Ser; for Gin (Q): Glu or Asn; for Glu (E): Gin or Asp; for Gly (G): Ala; for His (H): Asn, Gin, Lys, or Arg; for Ile (I): Val, Met, Ala, Phe, or Leu; for Leu (L): Val, Met, Ala, Phe, or lie; for Lys (K): Gin, Asn, or Arg; for Met (M): Phe, Ile, or Leu; for Phe (F): Leu, Val, Ile, Ala, or Tyr; for Pro (P): Ala; for Ser (S): Thr; for Thr (T): Ser; for Trp (W): Phe or Tyr; for Tyr (Y): Trp, Phe, Thr, or Ser; and for Val (V): Ile, Leu, Met, Phe, or Ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (see e.g., the PROWL website of Rockefeller University, New York, N.Y., United States of America). For solvent exposed residues, conservative substitutions would include: Asp and Asn; Asp and Glu; Glu and Gin; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (d.) Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one can also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

The presently disclosed subject matter is also directed to methods of administering the compounds of the presently disclosed subject matter to a subject.

Pharmaceutical compositions comprising the present compounds are administered to an individual in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

The presently disclosed subject matter is also directed to pharmaceutical compositions comprising the peptides of the presently disclosed subject matter. More particularly, such compounds can be formulated as pharmaceutical compositions using standard pharmaceutically acceptable carriers, fillers, solublizing agents and stabilizers known to those skilled in the art.

The presently disclosed subject matter also encompasses the use pharmaceutical compositions of an appropriate compound, homolog, fragment, analog, or derivative thereof to practice the methods of the presently disclosed subject matter, the composition comprising at least one appropriate compound, homolog, fragment, analog, or derivative thereof and a pharmaceutically-acceptable carrier.

The pharmaceutical compositions useful for practicing the presently disclosed subject matter can be administered to deliver a dose of between 1 ng/kg/day and 100 mg/kg/day. Pharmaceutical compositions that are useful in the methods of the presently disclosed subject matter can be administered systemically in oral solid formulations, ophthalmic, suppository, aerosol, topical or other similar formulations. In addition to the appropriate compound, such pharmaceutical compositions can contain pharmaceutically-acceptable carriers and other ingredients known to enhance and facilitate drug administration. Other possible formulations, such as nanoparticles, liposomes, resealed erythrocytes, and immunologically based systems can also be used to administer an appropriate compound according to the methods of the presently disclosed subject matter.

Compounds which are identified using any of the methods described herein can be formulated and administered to a subject for treatment of the diseases disclosed herein.

The presently disclosed subject matter encompasses the preparation and use of pharmaceutical compositions comprising a compound useful for treatment of the conditions, disorders, and diseases disclosed herein as an active ingredient. Such a pharmaceutical composition can consist of the active ingredient alone, in a form suitable for administration to a subject, or the pharmaceutical composition can comprise the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the presently disclosed subject matter can be prepared, packaged, or sold in formulations suitable for intratumoral, oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the presently disclosed subject matter can be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition can comprise between 0.1% and 100% (w/w) active ingredient.

In addition to the active ingredient, a pharmaceutical composition of the presently disclosed subject matter can further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter can be made using conventional technology. A formulation of a pharmaceutical composition of the presently disclosed subject matter suitable for oral administration can be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration can be prepared, packaged, and sold either in liquid form or in the form of a dry product intended for reconstitution with water or another suitable vehicle prior to use.

Liquid suspensions can be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions can further comprise one or more additional ingredients including, but not limited to, suspending agents, dispersing or wetting agents, emulsifying agents, demulcents, preservatives, buffers, salts, flavorings, coloring agents, and sweetening agents. Oily suspensions can further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents can be prepared in substantially the same manner as liquid suspensions, the primary difference being that the active ingredient is dissolved, rather than suspended in the solvent. Liquid solutions of the pharmaceutical composition of the presently disclosed subject matter can comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the presently disclosed subject matter can be prepared using known methods. Such formulations can be administered directly to a subject, used, for example, to form tablets, to fill capsules, or to prepare an aqueous or oily suspension or solution by addition of an aqueous or oily vehicle thereto. Each of these formulations can further comprise one or more of dispersing or wetting agent, a suspending agent, and a preservative. Additional excipients, such as fillers and sweetening, flavoring, or coloring agents, can also be included in these formulations.

A pharmaceutical composition of the presently disclosed subject matter can also be prepared, packaged, or sold in the form of oil in water emulsion or a water-in-oil emulsion. The oily phase can be a vegetable oil such as olive or arachis oil, a mineral oil such as liquid paraffin, or a combination of these. Such compositions can further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions can also contain additional ingredients including, for example, sweetening or flavoring agents.

A pharmaceutical composition of the presently disclosed subject matter can also be prepared, packaged, or sold in a formulation suitable for intratumoral administration, direct/topical administration, or parenteral administration

The pharmaceutical compositions can be prepared, packaged, or sold in the form of a sterile injectable aqueous or oily suspension or solution. This suspension or solution can be formulated according to the known art, and can comprise, in addition to the active ingredient, additional ingredients such as the dispersing agents, wetting agents, or suspending agents described herein. Such sterile injectable formulations can be prepared using a non-toxic parenterally acceptable diluent or solvent, such as water or 1,3 butane diol, for example.

Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems. Compositions for sustained release or implantation can comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi liquid preparations such as liniments, lotions, oil in water or water in oil emulsions such as creams, ointments or pastes, and solutions or suspensions. Topically-administrable formulations can, for example, comprise from about 1% to about 10% (w/w) active ingredient, although the concentration of the active ingredient can be as high as the solubility limit of the active ingredient in the solvent. Formulations for topical administration can further comprise one or more of the additional ingredients described herein.

A pharmaceutical composition of the presently disclosed subject matter can be prepared, packaged, or sold in a formulation suitable for pulmonary administration via the buccal cavity. Such a formulation can comprise dry particles which comprise the active ingredient and which have a diameter in the range in some embodiments from about 0.5 to about 7 nanometers and in some embodiments from about 1 to about 6 nanometers. Such compositions are conveniently in the form of dry powders for administration using a device comprising a dry powder reservoir to which a stream of propellant can be directed to disperse the powder or using a self propelling solvent/powder dispensing container such as a device comprising the active ingredient dissolved or suspended in a low-boiling propellant in a sealed container.

Such powders comprise particles wherein in some embodiments at least 98% of the particles by weight have a diameter greater than 0.5 nanometers and in some embodiments at least 95% of the particles by number have a diameter less than 7 nanometers. In some embodiments, at least 95% of the particles by weight have a diameter greater than 1 nanometer and in some embodiments at least 90% of the particles by number have a diameter less than 6 nanometers. Dry powder compositions can in some embodiments include a solid fine powder diluent such as sugar and are conveniently provided in a unit dose form.

Low boiling propellants generally include liquid propellants having a boiling point of below 65° F. at atmospheric pressure. Generally the propellant can constitute 50 to 99.9% (w/w) of the composition, and the active ingredient can constitute 0.1 to 20% (w/w) of the composition. The propellant can further comprise additional ingredients such as a liquid non-ionic or solid anionic surfactant or a solid diluent (in some embodiments having a particle size of the same order as particles comprising the active ingredient).

Pharmaceutical compositions of the presently disclosed subject matter formulated for pulmonary delivery can also provide the active ingredient in the form of droplets of a solution or suspension. Such formulations can be prepared, packaged, or sold as aqueous or dilute alcoholic solutions or suspensions, optionally sterile, comprising the active ingredient, and can conveniently be administered using any nebulization or atomization device. Such formulations can further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration in some embodiments have an average diameter in the range from about 0.1 to about 200 nanometers.

The formulations described herein as being useful for pulmonary delivery are also useful for intranasal delivery of a pharmaceutical composition of the presently disclosed subject matter.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration can, for example, comprise from about as little as 0.1% (w/w) and as much as 100% (w/w) of the active ingredient, and can further comprise one or more of the additional ingredients described herein.

A pharmaceutical composition of the presently disclosed subject matter can be prepared, packaged, or sold in a formulation suitable for buccal administration. Such formulations can, for example, be in the form of tablets or lozenges made using conventional methods, and can, for example, 0.1 to 20% (w/w) active ingredient, the balance comprising an orally dissolvable or degradable composition and, optionally, one or more of the additional ingredients described herein. Alternately, formulations suitable for buccal administration can comprise a powder or an aerosolized or atomized solution or suspension comprising the active ingredient. Such powdered, aerosolized, or aerosolized formulations, when dispersed, have an average particle or droplet size in some embodiments in the range from about 0.1 to about 200 nanometers, and can further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which can be included in the pharmaceutical compositions of the presently disclosed subject matter are known in the art and described, for example in Remington's Pharmaceutical Science. 18th ed., which is incorporated herein by reference.

Typically, dosages of the compound of the presently disclosed subject matter which can be administered to an animal, in some embodiments a human, range in amount from 1 μg to about 100 g per kilogram of body weight of the subject. While the precise dosage administered will vary depending upon any number of factors, including but not limited to, the type of animal and type of disease state being treated, the age of the animal and the route of administration. In some embodiments, the dosage of the compound will vary from about 10 μg to about 10 g per kilogram of body weight of the animal. In some embodiments, the dosage will vary from about 10 mg to about 1 g per kilogram of body weight of the subject.

The compound can be administered to a subject as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the subject, etc.

The presently disclosed subject matter also includes a kit comprising a compound of the presently disclosed subject matter and an instructional material which describes administering the composition to a cell or a tissue of a subject. In some embodiments, this kit comprises a solvent (in some embodiments, a sterile solvent) suitable for dissolving or suspending the composition of the presently disclosed subject matter prior to administering the compound to the subject. The presently disclosed subject matter also provides an applicator, and an instructional material for the use thereof.

Without further description, it is believed that one of ordinary skill in the art can, using the preceding description and the following illustrative examples, make and utilize the compounds of the presently disclosed subject matter and practice the claimed methods. The following examples therefore, specifically point out exemplary non-limiting embodiments of the presently disclosed subject matter, and are not to be construed as limiting in any way the remainder of the disclosure.

### EXAMPLES

The following Examples provide further illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Materials and Methods for Examples 1-7

Cell Culture and Reagents.

VMM39, VMM1, VMM18 human melanoma cell lines were established from metastatic lesions of patients at the University of Virginia (approved by the Institutional Review Board of the University of Virginia). DM93, DM331, DM13, and SLM2 melanoma cell lines had been established from metastatic lesions by Dr. H. F. Seigler at Duke University (Durham, N.C., United States of America; see Hogan et al., 2005; Huntington et al., 2004; Kittlesen et al., 1998; Molhoek et al., 2008; Slingluff et al., 1993; Yamshchikov et al., 2001; Yamshchikov et al., 2005). SK-MEL-2 and SK-MEL-28 melanoma cells were established in Memorial Sloan Kettering Cancer Center (New York, N.Y., United States of America) and obtained from the American Type Culture Collection (ATCC, Manassas, Va., United States of America). All melanoma cells were grown in RPMI media supplemented with 10% fetal bovine serum (FBS) and 1% penicillin/streptomycin (P/S). PIG1 and PIG3V melanocytes were described before (Le Poole et al., 2000) and maintained in Media 254 containing 1% of human melanocyte growth supplement (HMGS), 5% FBS, and 1% (P/S). All cells were grown at 37° C. in 5% C02. INVITROGEN™ brand Tissue Extraction Reagent I was obtained from INVITROGEN™ Corporation (Carlsbad, Calif., United States of America). Propidium iodide, 7-aminoactinomycin D (7-AAD), and BrdU kit were purchased from BD Biosciences (San Diego, Calif., United States of America). Vector Laboratories's IMMPRESS™ polymer kit for TMAs immunostaining was obtained from Vector Laboratories (Burlingame, Calif., United States of America). Pevonedistat and vemurafenib (PLX4032) were purchased from Active Biochem (Wan Chai, Hong Kong), and were dissolved in DMSO and used at the indicated doses.

Cell Lysis, SDS-PAGE, and Immunoblotting.

Melanoma cells were lysed with RIPA lysis buffer (50 mM Tris, pH 8.0; 150 mM NaCl, 1% NP-40; 0.5% sodium deoxycholate; 0.1% SDS; 1 mM Benzamidine-HCl; 0.5 μg/ml Leupeptin; 0.5 μg/ml Aprotinin; 1 μg/ml pepstatin; 20 mM NaF; 20 mM Na₃VO₄), and equal amounts of protein were electrophoretically separated in a polyacrylamide 8-12% gel (Bio-Rad Laboratories, Inc., Hercules, Calif., United States of America), trans-blotted to a nitrocellulose membrane, and incubated overnight with primary antibodies at 4° C. The following antibodies were used: Anti-p21 (C19), anti-p27 (C19), anti-p53 (DO-1), and anti-tubulin (10D8) were purchased from Santa Cruz Biotechnology, Inc. (California, United States of America). Antibodies against SET8, CHK1, CHK2, p-CHK1 (S375), p-CHK2 (T68), H2AX and p-H2AX (γH2AX; T139), and PARP were purchased from Cell Signaling Technology, Inc. (Danvers, Mass., United States of America). Anti-Cul3 was purchased from Bethyl Laboratories (Montgomery, Tex., United States of America). Anti-CDT1 and anti-CDT2 antibodies were described before (Abbas et al., 2010). The immunoblot signals were detected by enhanced chemiluminescence. For murine melanoma xenografts, tumors were isolated, washed three times with cold PBS, and frozen at −80° C. until use. Frozen specimens were ground in a dry-iced mortar and subsequently lysed in two volumes of Tissue Extraction Reagent I supplemented with protease and phosphatase inhibitors as set forth herein above. Tissue lysates were probed for different proteins by immunoblotting following the procedure described herein above.

RNA Interference (siRNA)-Mediated Gene Silencing.

siRNA transfections were performed using LIPOFECTAMINE® RNAimax brand transfection reagent according to the manufacturer's protocol (INVITROGEN™, Carlsbad, Calif., United States of America). Cells were seeded at 30% confluency and transfected with the individual siRNAs (10 nM each) in RPMI media supplemented with 10% fetal bovine serum (FBS) and 1% penicillin/streptomycin (P/S). In co-knockdown experiments, DM93 or VMM39 cells were transfected with the individual siRNAs (10 nM each with 10 nM control siGL2- for normalization) or siRNAs targeting CDT1, SET8, or p21 along with siRNA targeting CDT2 (10 nM each—total 20 nM siRNAs). Control cells were transfected with 20 nM si-GL2. Cells were harvested 72 hours post-transfection for cell cycle analysis or at 96 hours for β-gal staining. The siRNAs employed are summarized in Table 2.

TABLE 2
siRNA Targets and Nucleotide Sequences

| Construct | Sense Strand Nucleotide Sequence |
|---|---|
| si-GL2 | 5′-AACGUACGCGGAAUACUUCGA-3′ |
| | (SEQ ID NO: 1) |
| si-CDT2 | 5′-GAAUUAUACUGCUUAUCGA-3′ |
| | (SEQ ID NO: 2) |
| si-CDT1 | 5′-AACGUGGAUGAAGUACCCGAC-3′ |
| | (SEQ ID NO: 3) |
| si-SET8 | 5′-GAUUGAAAGUGGGAAGGAA-3′ |
| | (SEQ ID NO: 4) |
| si-p21 | 5′-AACAUACUGGCCUGGACUG-3′ |
| | (SEQ ID NO: 5) |
| si-Geminin: | 5′-UGCCAACUCUGGAAUCAAA-3′ |
| | (SEQ ID NO: 6) |
| si-Emi1-1* | 5′-GAGAAUUUCGGUGACAGUCUA-3′ |
| | (SEQ ID NO: 7) |
| si-Emi1-2 | 5′-UACGAAGUGUCUCUGUAAUUA-3′ |
| | (SEQ ID NO: 8) |

*see Machida & Dutta, 2007

2.4. Gene Targeting by CRISPR/Cas9.

Single guide-RNAs (sgRNAs) targeting the DTL (sg-CDT2-1 and sg-CDT2), SET8, and CDKN1A genes were cloned into pX330 vector containing a human codon-optimized SpCas9 endonuclease (Catalogue No. 42230, Addgene, Cambridge, Mass., United States of America) using BbsI restriction enzyme cutting sites, and transfected in the various cell lines. After puromycin selection, cells were seeded to obtain single colonies. Genomic DNA was extracted using 100 mM NaCl, 50 mM Tris-HCl pH 7.0, 5 mM EDTA and 1% SDS. Genotyping was performed using PCR amplification of genomic DNA using the following forward and reverse primer sets, respectively. For CDT2: 5′-TGTTGTGAGAGGCGCAAGCTGC-3′ (SEQ ID NO: 9) and 5′-GGTCGGAGGTGGCGTGTGTTTC-3′ (SEQ ID NO: 10); for SET8: 5′-GTCTTTCCCCCACCTCCGCCTG-3′ (SEQ ID NO: 11) and 5′-CTTTTTTCGGGGGGCCTGTTTGC-3′ (SEQ ID NO: 12), for p21: 5′-TCACCTGAGGTGACACAGCAAAGC-3′ (SEQ ID NO: 13) and 5′-GGCCCCGTGGGAAGGTAGAGCTT-3′ (SEQ ID NO: 14). Targets of the various sgRNAs are as follows: For DTL (CDT2): 5′-GCACCGAATTGAAGAGCATC-3′ (for sg-CDT2-1; SEQ ID NO: 15); and 5′-CATTTCTCAGGACGCCAAGC-3′ (for sg-CDT2-2; SEQ ID NO: 16); for SET8: 5′-ACGGAGCGCCATGAAGTCCG-3′ (SEQ ID NO: 17); for CDKNIA: 5′-GCGCCATGTCAGAACCGGCT-3′ (SEQ ID NO: 18). Insertions/deletions (Indels) identification was performed using SURVEYOR® Mutation Detection Kit according to the manufacturer's protocol (Integrated DNA Technologies, Inc., San Diego, Calif., United States of America). For sequencing, PCR amplified gene products were cloned into TOPO® TA Vector using TOPO® TA CLONING® Kit according to the manufacturer's instructions (INVITROGEN™, Carlsbad, Calif., United States of America) and transformed into DH5α. Plasmids were retrieved by the QIAPREP® Spin Miniprep Kit (Qiagen Inc., Valencia, Calif., United States of America) and confirmed by sequencing (Eurofins Scientific, Louisville, Ky., United States of America).

Cell Proliferation/Viability Assays and Washout Experiments.

Proliferation and viability of cultured cells was measured by CELLTITER96® Non-radioactive Cell Proliferation Assay (Promega Corporation; Madison, Wis., United States of America). Briefly, various wild type and mutant BRAF melanoma cells were seeded in 96 well plates and treated with pevonedistat, vemurafenib, or the combination pevonedistat and vemurafenib at various concentrations. Control cells were treated with DMSO. 96 hours following treatment, cells were stained with the dye solution according to the manufacturer's protocol. Absorbance was recorded at 570 nm and growth curves were established. To test the effect of transient exposure of melanoma cells to pevonedistat on rereplication and growth inhibition, washout experiments were conducted where melanoma cells or PIG3V melanocytes were treated with 1 M pevonedistat for different times (4, 8, 12, and 24 hours) before the drug was washed out by washing the cells twice with PBS, and adding drug-free fresh growth media to cells. Cells were counted every 24 hours using a COUNTESS™ Automated Cell Counter (INVITROGEN™, Carlsbad, Calif., United States of America), and harvested at the indicated times for PI staining and FACS analysis (cell cycle profile) or for immunoblotting.

Clonogenic Survival Assays.

Cell survival following CDT2 depletion or pevonedistat treatment was assessed by clonogenic survival assay, preformed in triplicates. 72 hours following transfection with si-GL2 or si-CDT2, cells were trypsinized, counted, and seeded in 60 mm dishes. For pevonedistat treatments, cells were counted and seeded in 60 mm dishes and treated 24 hours later with various doses of pevonedistat or with DMSO. Cells were cultured for two weeks and were subsequently washed in cold PBS, fixed in cold methanol for 10 minutes, and stained with crystal violet (0.5%) for 10 minutes. Plates were washed with water, dried, and pictures were captured using IMAGE LAB™ software (BioRad Laboratories, Inc., Hercules, Calif., United States of America). Quantification of colonies was performed using QUANTITY ONE® software (BioRad Laboratories, Inc., Hercules, Calif., United States of America). Results are represented as mean±s.e.m. of triplicates normalized to the corresponding DMSO-treated or si-GL2 transfected controls.

Senescence-Associated β-galactosidase Assays.

Senescence was monitored using β-galactosidase (β-gal) staining. Following the various treatments, cells were washed twice with PBS, fixed with 2% formaldehyde/0.2% gluteraldehyde in PBS for 15 minutes at room temperature, and washed twice with PBS. The cells were stained with fresh X-Gal solution (1 mg/ml X-gal, 40 mM C₆H₈O₇.H₂O, 5 mM K₃Fe(CN)₆, 5 mM K₄Fe(CN)₆.3H₂O, 150 mM NaCl, and 2 mM MgCl₂.6H₂O in PBS) for 3-12 hours at 37° C. in the dark. Cells were washed three times in PBS and fixed with 100% methanol for 5 minutes at room temperature. Bright field blue color images were taken with an AMG EVOS® XL Core Imager/camera microscope (Life Technologies, Inc., Carlsbad, Calif., United States of America) counting at least 100 cells from at least 3 fields.

Flow Cytometry Analysis.

The effects of pevonedistat, vemurafenib, and/or silencing of various cell cycle-associated proteins by siRNA on cell cycle distribution and rereplication were assessed by propidium iodide staining and flow cytometry of asynchronous melanoma cultures. Synchronization of cells was not employed to avoid bias and to be able to measure the impact of these perturbations on proliferating cancer cells. Briefly, asynchronous melanoma cell lines were treated with pevonedistat or vemurafenib, or transfected with si-CDT1, si-CDT2, si-SET8, si-p21, sigeminin, si-EMI1, or si-GL2 for a time ranging from 24 to 96 hours. Cells were washed with cold PBS, harvested, and fixed in 70% (v/v) ethanol. Cells were subsequently treated with 20 μg of DNase-free RNase and stained with propidium iodide according to instructions of the manufacturer. Samples were analyzed on a FACSCAN™ (Becton, Dickinson and Company, Franklin Lakes, N.J., United States of America) and G0-G1, S, and G2-M fractions were segmented, and apoptotic (sub-G1 DNA content) and rereplicating (>G2/M DNA content) fractions were determined using FLOWJO® (FLOWJO, LLC, Ashland, Oreg., United States of America) and ModFit (Verity Software House, Topsham, Me., United States of America) software.

Bromodeoxy Uridine (BrdU) Staining and Flow Cytometry.

The effects of pevonedistat and/or silencing of cell cycle-associated proteins on cell cycle distribution or rereplication were assessed by flow cytometry according to the manufacturer's instructions. Different melanoma lines were transfected with si-GL2, si-CDT2, si-CDT1, si-SET8, si-p21, or si-geminin for a time ranging from 24 to 96 hours. At the end of treatment, cells were pulsed with BrdU (10 nM) for 1 hour in the dark prior to harvesting. Cells were washed with PBS and staining solution before the fixation and permeabilization steps according to the manufacturer's instructions. Cells were subsequently stained with anti-BrdU antibody solution for 20 minutes at room temperature, washed, and stained with 7-AAD solution for 30 minutes at 4° C. The cells were resuspended in 1 ml of staining buffer and kept overnight at 4° C. before analysis. Samples were analyzed on a FFACSCAN™ (Becton, Dickinson and Company, Franklin Lakes, N.J., United States of America), and different phases of the cell cycle were determined using FLOWJO® (FLOWJO, LLC, Ashland, Oreg., United States of America) and ModFit (Verity Software House, Topsham, Me., United States of America) software.

Staining and Analysis of Melanoma Tissue Microarray (TMA).

Formalin-fixed paraffin-embedded (FFPE) tissue blocks were retrieved from archives of the Department of Pathology, University of Virginia (UVA), Charlottesville, Va., United States of America. Use of human tissues was approved by the UVA Institutional Review Board (protocol 10598). Hematoxylin and eosin (H&E) slides from each block were reviewed by a pathologist to identify tumor areas. TMAs were constructed with 1.0-mm diameter tissue cores from representative tumor areas from the FFPE tissue blocks, transferred into a recipient paraffin block using a semi-automated tissue array instrument (TMARRAYER™; Pathology Devices, Inc., Westminster, Md., United States of America). Quadruplicate or triplicate tissue cores were taken from each specimen, resulting in nine (9) composite TMA blocks containing tissue cores from 18 to 27 specimens each. Control tissues from spleen, liver, placenta, and kidney were included in each TMA block. Multiple 4 μm sections were cut for H&E and immunohistochemical staining. The human melanoma tissue microarray (TMA) was evaluated for expression of CDT2 and Ki67 by immunohistochemistry. Details of this TMA have been reported previously (Erdag et al., 2012). These arrays included surgical specimens of human melanoma. Protein expression patterns of CDT2 and Ki67 were assessed in 138 tumor specimens in the TMA. Three nevi were used as a control. Antigen-retrieval step was performed at low pH 0.01% citric acid for 20 minutes at 100° C. Endogenous peroxidase was blocked using Bloxall (Catalogue No. SP-6000, Vector Laboratories, Inc., Burlingame, Calif., United States of America) for CDT2 detection and 0.3% Hydrogen peroxide for Ki67 detection; for 10 minutes; prior to serum blocking for 20 minutes, at room temperature. Incubation with CDT2 primary antibody (Abbas et al., 2008; 1:100 dilution) was performed at room temperature for 30 minutes. Staining with Ki67 primary antibody (Vector Laboratories, Inc., Burlingame, Calif., United States of America; 1:50 dilution) was performed overnight at 4° C. Omitting the primary antibody served as a negative control for the staining. The secondary antibody (Catalogue No. SK-4200; IMMPRESS™ Reagent, Vector Laboratories, Inc., Burlingame, Calif., United States of America; 1:500 dilution) was used for 30 minutes followed by substrate 3-amino-9-ethylcarbazole (AEC; Vector Laboratories, Inc., Burlingame, Calif., United States of America) incubation for 20 minutes, at room temperature as per the kit's instructions. Diaminobenzidine was utilized as the final chromogen and hematoxylin as the nuclear counterstain. Staining frequency of CDT2 and Ki67 were quantified manually by counting the number of positively stained nuclei in an average of three fields per core. The frequency was calculated by dividing the number of positive staining over the total number of cells in the same fields.

Immunohistochemical staining for BRAF mutation (V600E) was performed at the University of North Carolina, using Leica's Bond autostainer (Leica Biosystems, Nussloch, Germany) and the BRAF V600E antibody (clone VE1, dilution 1:400; Spring Bioscience Corporation, Pleasanton, Calif., United States of America). Mutational status is assessed by the presence or absence of staining in each core. Tumors with borderline staining and those with discrepant expression in between cores were excluded. The consensus value of the 2-4 representative cores from each tumor/patient sample arrayed was used for scoring and statistical analyses. TMA slides were quantified using Aperio ImageScope V11.2 (Leica Biosystems, Nussloch, Germany).

Kaplan-Meier Plot Analysis.

Publicly available TCGA data at cBioPortal (Cerami et al., 2012; Gao et al., 2013) was used to plot Kaplan-Meier plots on tumors divided into two groups based on level of CDT2 expressed as a Z-score (Collisson et al., 2014; Taylor et al., 2010; Weinstein et al., 2014).

Tumor Xenograft Studies.

All animal experiments were performed after approval of animal protocols by the Animal Care and Use Committee (ACUC) of the University of Virginia. Animals were housed and handled in accordance with the guidelines of the ACUC of the University of Virginia. The effect of pevonedistat on melanoma growth was tested in flank xenograft models. Foxn1^{nu} (20-25 g body weight, 4-5 weeks old females) athymic nude immune-deficient mice (Harlan Laboratories, now Envigo, Indianapolis, Ind., United States of America) were used in this study. Pevonedistat was dissolved in sterile 10% DMSO containing PBS (stock 1 mM), filtered, and stored in −20° C. until use. DM93, VMM39, SLM2, DM331, or SK-MEL-24 (2×10⁶) melanoma cells were implanted in both flanks of immune-deficient mice (n=12 mice per group) and tumor growth was monitored until they grow to an average volume of 150-200 mm³, and mice were randomized into groups for treatment. Animals were administered 0.2 mL pevonedistat solution (30 or 60 mg/kg body weight as indicated) intraperitoneally on a schedule of two cycles of five-day treatment followed by five treatment-free days, for 3 weeks, or more as indicated. Control animals were treated with an equal volume of sterile vehicle (10% DMSO in PBS). Where indicated, mice received control rodent diet, or rodent diet with 417 mg/kg PLX4720 (Research Diets, Inc. New Brunswick, N.J., United States of America). Tumors were measured with an electronic caliper every other day for 3 weeks post-drug injection. Animal weight was recorded once a week to detect any weight loss due to the toxicity of drug treatment or tumor burden. At the end of the treatment, animals were euthanized and tumors harvested for further processing. The results shown are mean tumor volumes±s.e.m; *p<0.05, **p<0.01, ***p<0.001.

Statistical Analysis.

All experiments were performed in triplicate. Numerical data were expressed as mean±standard deviation (SD). Where applicable, data are presented as the mean±s.e.m. Two group comparisons were analyzed by two-sided Student's t-test. p values were determined for all analyses and p<0.05 was considered significant. Synergy was determined using the Bliss model of independence (Bliss, 1939; Fitzgerald et al., 2006). For correlations, a Spearman correlation was used and p values <0.05 were considered statistically significant.

### Example 1

### CDT2 was Overexpressed in Melanoma and its Elevated Expression Predicted Poor Patient Outcome

Melanoma is one of the few cancers in which the genetic predisposition is to a large extent associated with mutations in the machinery of DNA replication (e.g. CDKN2A), but thus far most successful targeted drugs have focused on the oncogenic drivers in the MAP Kinase pathway (Lovly & Shaw, 2014). Therefore, it was suspected that vulnerabilities to drugs targeting the DNA replication machinery would be identified, and thus, searched gene expression databases for alterations in genes controlling DNA replication in melanoma. Using mRNA expression in a publicly available database of cutaneous melanomas (Talantov et al., 2005), it was found that CDT2 mRNA expression is elevated about three-fold in 84% of melanomas compared with normal skin (p=1.35×10⁻⁵) or with benign skin nevi (p=4.0×10⁻⁸). Employing data output from the Oncomine mRNA databases (see D'Errico et al., 2009; Hou et al., 2010; Roessler et al., 2010; Sun et al., 2006; and Zhai et al., 2007), CDT2 was also determined to be overexpressed in malignancies of the breast (invasive breast carcinoma vs. normal: p=1.91×10⁻³³; t-test=16.072; fold change=6.138), cervix (cervical squamous cell carcinoma epithelia vs. normal: p=2.44×10⁻¹⁰; t-test=11.429; fold change=5.550), stomach (gastric intestinal type adenocarcinoma vs. normal: p=295×10⁻¹³; t-test=9.373; fold change=3.631), lung (squamous cell lung carcinoma vs. normal: p=3.11×10⁻²⁵; t-test=14.693; fold change=6.913), liver (hepatocellular carcinoma vs. normal: p=7.92×10⁻⁷⁷; t-test=25.497; fold change=4.848), and brain malignancies (glioblastoma vs. normal: p=1.63×10⁻²³; t-test=13.396; fold change=6.171). CDT2 overexpression in melanoma was specific, as changes in the normalized expression levels of other components of the CRL4 E3 ligase (Cul4A, Cul4B and Rb×1), or four (4) other CRL4 substrate receptors (i.e. DDB2, VPRBB1, DWR68, and DCAF8) were not detected in microarrays of cutaneous melanomas (n=45) compared to benign melanocytic skin nevi (n=18; Talantov et al., 2005).

Next, the expression of CDT2 in human cutaneous melanoma was examined using data obtained from The Cancer Genome Atlas (TCGA) project available at cBioPortal (Cerami et al., 2012; Gao et al., 2013). This data set contains RNA expression data from 471 primary and metastatic melanomas from 468 patients. CDT2 expression was stratified into high and low expressers based the median expression of CDT2 (0.23 RSEM). Kaplan-Meier plots revealed that tumors with higher CDT2 expression (CDT2 level >0.23z; n=168) correlated with significantly lower probability of overall survival (median of 61.47 months vs. 151.15 months; p=4.3×10⁻⁴) than tumors with lower CDT2 expression (CDT2 level <0.23z; n=176). Tumors with higher CDT2 expression (CDT2 level >0.23z; n=156) also correlated with significantly lower probability of disease free survival (median of 44.65 months vs. 72.8 months; p=8.65×10⁻³) than tumors with lower CDT2 expression (CDT2 level <0.23z; n=153).

Kaplan-Meier plots of overall survival or disease free survival for the human skin cutaneous melanoma (SKCM) from TCGA stratified by CDT2 median expression level in a subset of melanoma for which mutational analysis was available (278 samples in total) exhibited similar correlations between CDT2 expression and overall survival (High: CDT2 level >0.38z; n=132; median survival in months=61.47; Low: CDT2 level <0.38z; n=126: median survival in months=161.96; p=1.8×10⁻⁴) and disease free survival (High: CDT2 level >0.38z; n=115; median disease free survival in months=48; Low: CDT2 level <0.38z; n=115: median disease free survival in months=72.8; p=0.0358).

Analysis of the BRAF or NRAS mutations in these tumors showed modest increase in the probability of overall survival in patients with BRAF mutations (mutant BRAF, n=136, median survival in months=127.1; wild-type BRAF, n=122; median survival in months=68.1; p=0.0353), and no significant correlations in patients with NRAS mutations (mutant NRAS, n=91, median survival in months=65.87; wild-type NRAS, n=167; median survival in months=113.44; p=0.317). Further analysis demonstrated that 72% of tumors with high CDT2 expression harbored either BRAF (31%) or NRAS (41%) mutations. This was not significantly different in low CDT2 expressing tumors, with 74% of these tumors containing BRAF (52%) or NRAS (21%) mutations. Gene co-expression analysis demonstrated that CDT2 elevated expression correlated with the expression of several E2F1 target genes, suggesting that CDT2 overexpression in melanoma could have been due to increased E2F1 transcriptional activity, which has been shown to promote transcription from the CDT2 (DTL) promoter (Nakagawa et al., 2008).

These results suggested that elevated CDT2 expression did not correlate with BRAF/NRAS mutational status. The higher percentage of NRAS mutations in melanomas with high CDT2 expression compared to the higher percentage of BRAF mutations in melanomas with low CDT2 expression suggested an interaction between CDT2 expression and activated NRAS, although it remained possible that this distribution could have reflected therapeutic response in the BRAF melanoma patients.

CDT2 protein expression in a human tissue microarray (TMA) comprising 138 melanoma specimens from 100 patients (42 female, 58 male, ages 23-90; mean 59±16 years) was also examined. These include eight (8) patients with large primary cutaneous melanoma and 92 with one or more metastatic melanomas. CDT2 protein was predominantly nuclear and significantly elevated in 84.7% of all melanomas (117/138), whereas CDT2 was not detectable in non-malignant melanocytes (CDT2 composite expression score in cutaneous melanoma TMA, 138 melanoma specimens from 100 patients, compared to non-malignant nevi: p<0.001, calculated using Student's t-test). CDT2 expression however, varied significantly in melanomas, with metastatic melanomas exhibiting higher expression compared to primary tumors (relative CDT2 composite expression score in metastatic melanoma compared to primary melanoma: p<0.05). Analysis of Ki67 staining demonstrated a modest, but statistically significant, correlation between CDT2 and Ki67 staining (r=0.447, p<0.01). In this in situ analysis, a correlation between CDT2 expression and the BRAF mutational status, disease stage, or with lymphocytic infiltration was not observed (p>0.05), nor was there a correlation with other parameters such as age, tissue type, gender, or patient survival. The lack of correlation with patient survival in this data set can be explained by its small size and the fact that these tumors were mostly metastatic. Together, these results demonstrated that CDT2 expression was elevated in melanoma and served as a negative prognostic marker for the disease.

### Example 2

### CDT2 was Required for Melanoma Cell Proliferation

Although CDT2 is overexpressed in melanoma and in other cancers, it is not likely to function as a classical oncogene. Instead, it appears to act as a cancer-associated gene to which cancer cells become “addicted”. This is reminiscent to the secondary physiological changes that stress cellular capacity for survival as a consequence of oncogenic activation, common in melanoma and in other cancers; the so called “non-oncogene addiction” (Luo et al., 2009).

It was thus hypothesized that CDT2 is overexpressed in melanoma cells to alleviate replication stress that can be induced by melanoma oncogenes. To test this hypothesis, the expression of CDT2 was silenced by siRNA in a panel of melanoma lines with various genetic mutations including the BRAF mutant DM93 cells (see Table 3). 4×10⁶ cells of each cell line were seeded at the beginning of transfection, and cells were tested 96 hours following transfection with si-CDT2 (SEQ ID NO: 2) or control si-GL2 (SEQ ID NO: 1) as described herein above in the Materials and Methods for the EXAMPLES section.

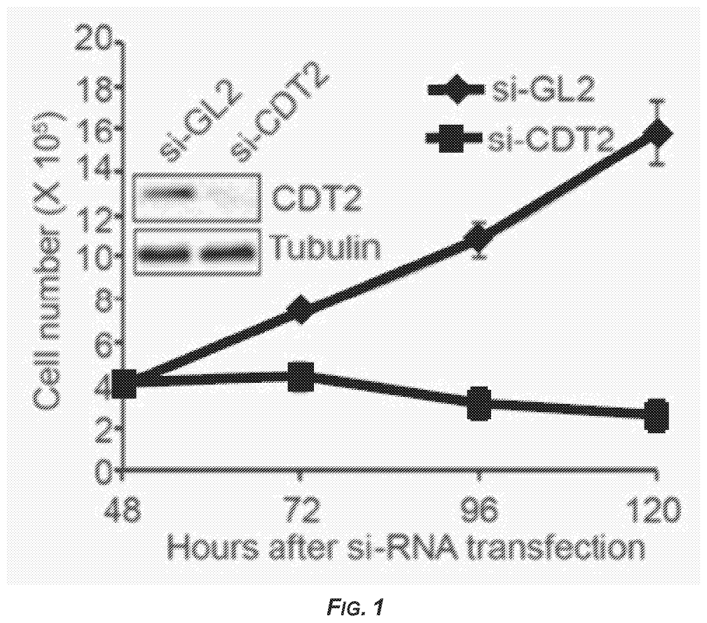

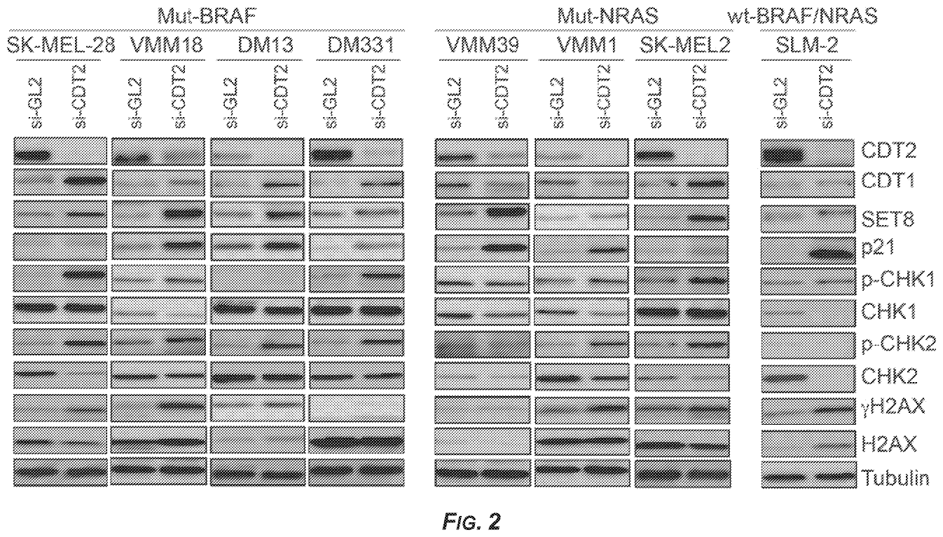

Depletion of CDT2 by siRNA (FIGS. 1 and 2) suppressed melanoma cell proliferation and induced morphological changes associated with rereplication: flattening of cells and increase in nuclear size. FACS analysis confirmed the increase in cells with >4N DNA content (47.8% vs. 0.726%), as well as a small but reproducible increase in cells with sub-G1 DNA content (4.8% vs. 1.15%) indicative of apoptosis. Bromodeoxy uridine (BrdU) incorporation and FACS analysis further illustrated that CDT2 knockdown resulted in rereplication (57.2% vs. 0.55%) during the same cell cycle. Inhibition of proliferation and DNA rereplication were also observed following CDT2 knockdown in a panel of nine (9) melanoma cell lines with various mutations (Table 3), including VMM39 cells with NRAS and PDGFRA activating mutations (FIG. 2). DNA rereplication, morphological changes and suppression of proliferation were also observed in five (5) melanoma lines following the deletion of CDT2/DTL gene using CRISPR/Cas9 and two different single guide RNAs (sg-RNAs) that target two different regions in exon 1 in CDT2 (sg-CDT2-1 (SEQ ID NO:15): 18.4% rereplication; sg-CDT2-2 (SEQ ID NO: 16): 22.6% rereplication; control

TABLE 3
Summary of Mutations Present in Melanoma Cell Lines

| | Cell line | | | | | | | | | | |
|---|---|---|---|---|---|---|---|---|---|---|---|
| Gene Name | DM_13 | DM331 | DM93 | PIG1 | PIG3V | SKMel2 | SkMel28 | SLM_2 | VMM_1 | VMM_18 | VMM_39 |
| ABL1 | | | | | | | | | | | |
| AKT1 | | | | | | | n.d. | | | | |
| APC | wt/? | | wt/? | | wt/? | wt/? | wt/? | | wt/? | wt/? | wt/? |
| BRAF | mutant | mutant | mutant | | | | mutant | | | mutant | |
| CDKN2A | mutant | wt/? | wt/? | wt/? | wt/? | wt/? | mutant | wt/? | mutant | mutant | wt/? |
| CSF1R (FMS) | | | | | | | | | | | |
| CTNNB1 | wt/? | wt/? | wt/? | wt/? | wt/? | wt/? | wt/? | wt/? | wt/? | wt/? | wt/? |
| EGFR | wt/? | wt/? | wt/? | wt/? | wt/? | wt/? | wt/? | wt/? | wt/? | wt/? | wt/? |
| ERBB2 | wt/? | wt/? | wt/? | wt/? | wt/? | wt/? | wt/? | wt/? | wt/? | wt/? | wt/? |
| FGFR1 | | | | | | | | | | | |
| FGFR2 | | | | | | | | | | | |
| FGFR3 | wt/? | wt/? | wt/? | wt/? | wt/? | wt/? | wt/? | wt/? | wt/? | wt/? | wt? |
| FLT3 | wt/? | wt/? | wt/? | wt/? | wt/? | wt/? | wt/? | wt/? | wt/? | wt/? | wt/? |
| HRAS | | | mutant | | | | wt/? | | | | wt/? |
| JAK2 | | | | | | | | | | | |
| JAK3 | wt/? | wt/? | wt/? | wt/? | wt/? | wt/? | wt/? | wt/? | wt/? | wt/? | wt/? |
| KIT | | | | | | | wt/? | | | | |
| KRAS | wt/? | wt/? | wt/? | wt/? | wt/? | wt/? | wt/? | wt/? | wt/? | wt/? | wt/? |
| MET | | | | | | | | | | | |
| MLH1 | | | | | | | | | | | |
| MYC (C-MYC) | | | | | | | wt/? | | | | |
| NRAS | | | | | | mutant | wt/? | | mutant | | mutant |
| PDGFRA | wt/? | wt/? | mutant | wt/? | mutant | wt/? | wt/? | wt/? | wt/? | wt/? | mutant |
| PIK3CA | wt/? | wt/? | wt/? | wt/? | wt/? | wt/? | wt/? | wt/? | wt/? | wt/? | wt/? |
| PTEN | | | | | | | wt/? | | | | |
| RB1 | | | | | | | wt/? | | | | |
| RET | wt/? | wt/? | wt/? | wt/? | wt/? | wt/? | wt/? | wt/? | wt/? | wt/? | wt/? |
| STK11 | | | | | | | wt/? | | | | |
| TP53 | | | | | | mutant | wt/? | | | | |
| VHL | | | | | | | | | | | |

wt: wild-type;
wt/?: wild-type/unknown;
mutant: mutant;
n.d.: not determined

sg-RNA (transfection of DM93 T cells with pX330 vector containing a human codon-optimized SpCas9 endonuclease (Catalogue No. 42230, Addgene, Cambridge, Mass., United States of America) but without sg-RNA): 1.88% rereplication.

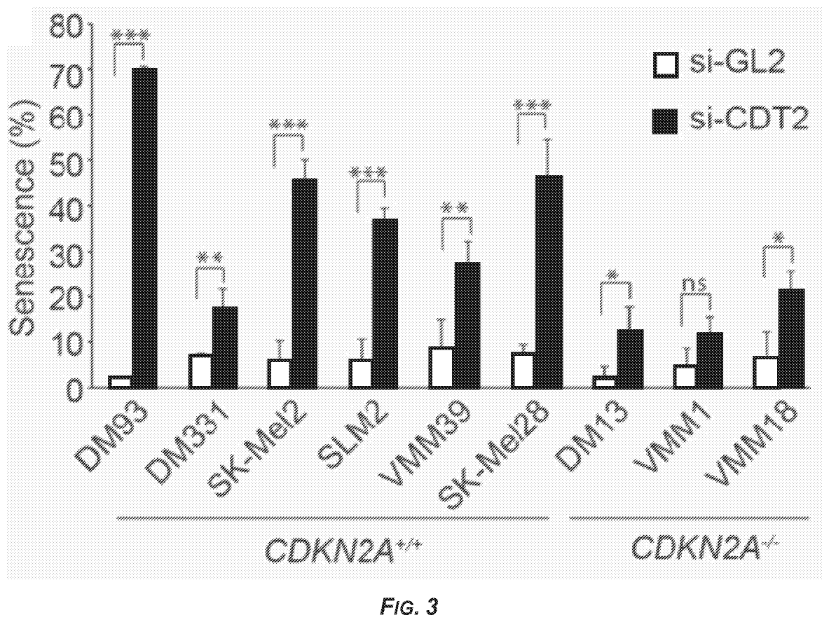

CDT2 knockdown in DM93 and in other melanoma cells increased the steady state level of the CRL4^{CDT2} ubiquitylation substrates SET8 and p21, but elevated CDT1 was only noted in some, but not all, melanoma lines (FIG. 2). This was associated with spontaneous DNA damage (increased γH2AX), induction of DNA damage checkpoint (phosphorylation of the checkpoint proteins CHK1 and CHK2), senescence (as detected by increased β-galactosidase (β-gal) staining), and apoptosis (PARP cleavage; see FIGS. 2 and 3). Thus, CDT2 depletion or deletion inhibited melanoma with various genetic mutations and this was accompanied by DNA rereplication, spontaneous DNA damage, and senescence.

### Example 3

### SET8 and p21 Promoted Rereplication and Senescence in CDT2-Depleted Melanoma Cells

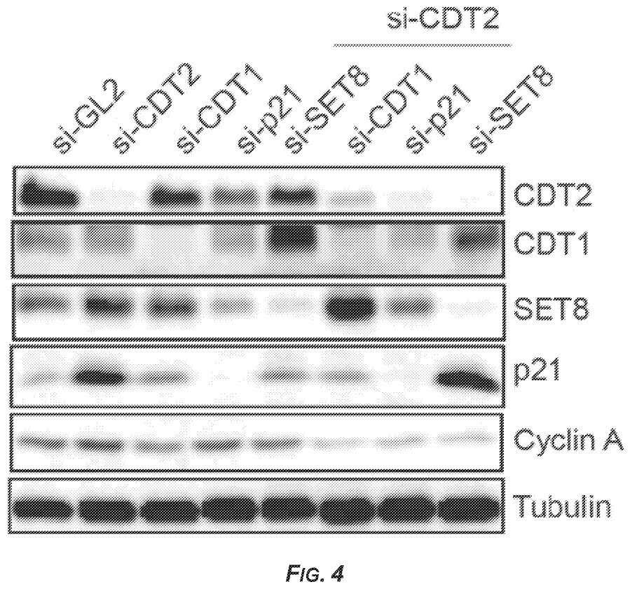

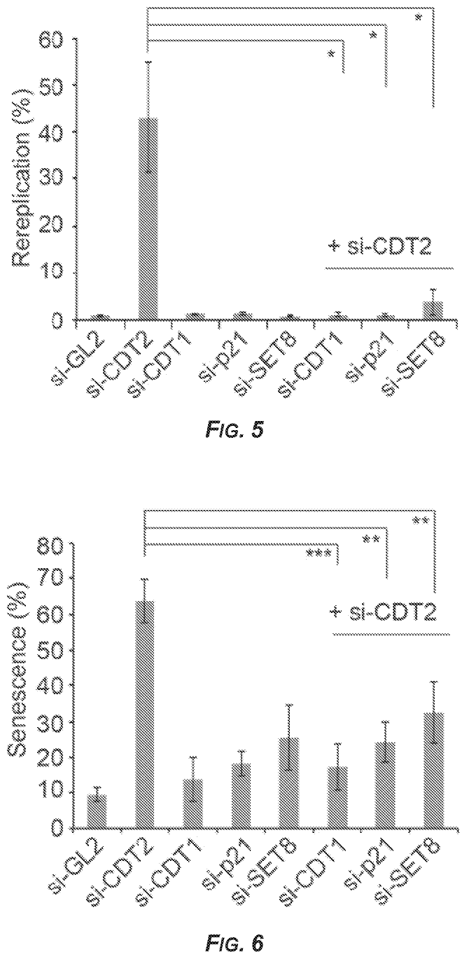

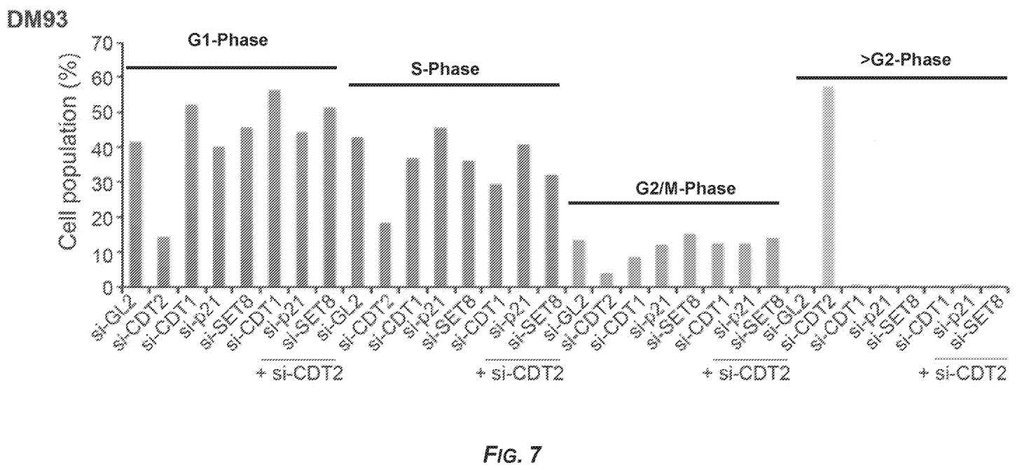

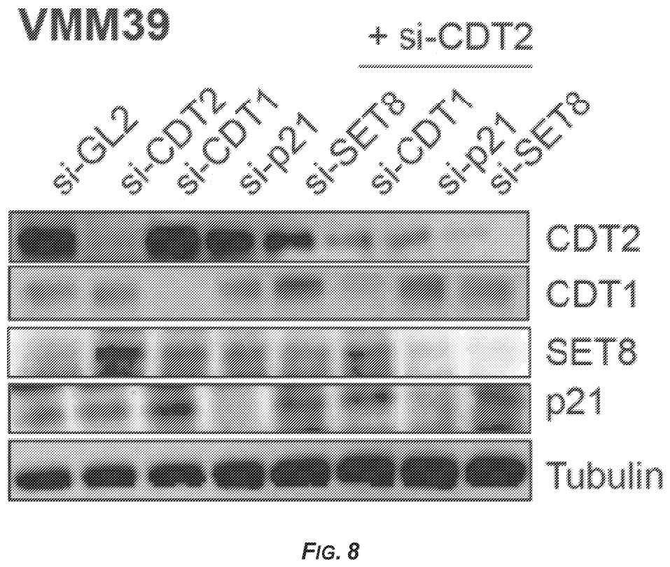

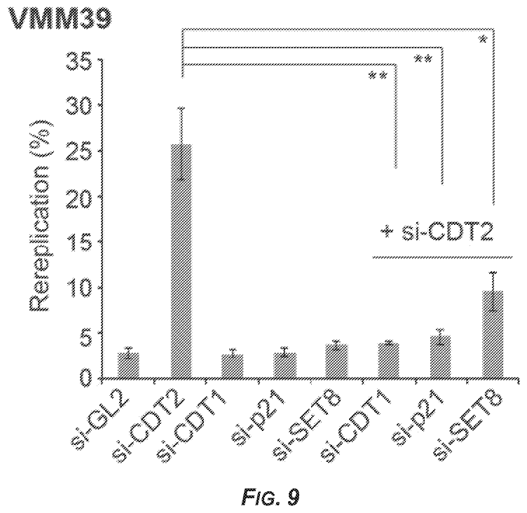

To investigate the mechanism by which CDT2 knockdown induced rereplication and senescence in melanoma cells, the expression of various substrates of CRL4^{CDT2} previously implicated in DNA rereplication (Abbas & Dutta, 2011; Abbas et al., 2013) was co-silences along with CDT2, both in DM93 and VMM39 cells. Silencing of CDT1 prevented both rereplication and senescence induced by CDT2 depletion, but increased the percentage of cells in G1 and decreased S-phase cells (FIGS. 4-9), suggesting that inhibition of rereplication is secondary to cell cycle bock in G1. Depletion of SET8 or p21 on the other hand, completely suppressed si-CDT2-induced rereplication and senescence without significantly impacting cell cycle distribution (FIGS. 4-9), suggesting that inhibition of rereplication was secondary to cell cycle block in G1. Thus, CDT1, SET8, and p21 were all required for si-CDT2-induced rereplication and senescence, but CDT1 appeared to be required primarily for entry into S-phase.

To examine the role of CDT1 further, various melanoma lines (DM93, VMM39, or VMM18) were depleted of geminin, an inhibitor of CDT1 whose depletion induces rereplication in a number of cancer cell lines (Zhu & Depamphilis, 2009). Interestingly, although si-geminin efficiently induced rereplication in the U20S osteosarcoma cells and in the Cal27 squamous cell carcinoma cells, it failed to do so in melanoma cells (FIG. 10). In contrast, depletion of EMI1 (an inhibitor of the APC ubiquitin ligase depletion of which results in the ubiquitin-dependent proteolysis of geminin and cyclin A; see Machida & Dutta, 2007) with either of two siRNAs, si-EMI1-1 (SEQ ID NO: 7) or si-EMI1-2 (SEQ ID NO: 8), induced robust rereplication in melanoma cells (see Table 4 below)

TABLE 4
Depletion of EMI1 Induced Rereplication
in DM93 and VMM39 Melanoma Cell Lines

| | DM93 | VMM3 |
|---|---|---|
| | Percent | Percent |
| siRNA | Rereplication | Rereplication |
| si-GL2 (negative control; SEQ ID NO: 1) | 0.246 | 2.39 |
| si-EMI1-1 (SEQ ID NO: 7) | 60.1 | 39.7 |
| si-EMI1-2 (SEQ ID NO: 8) | 90.3 | 54.2 |

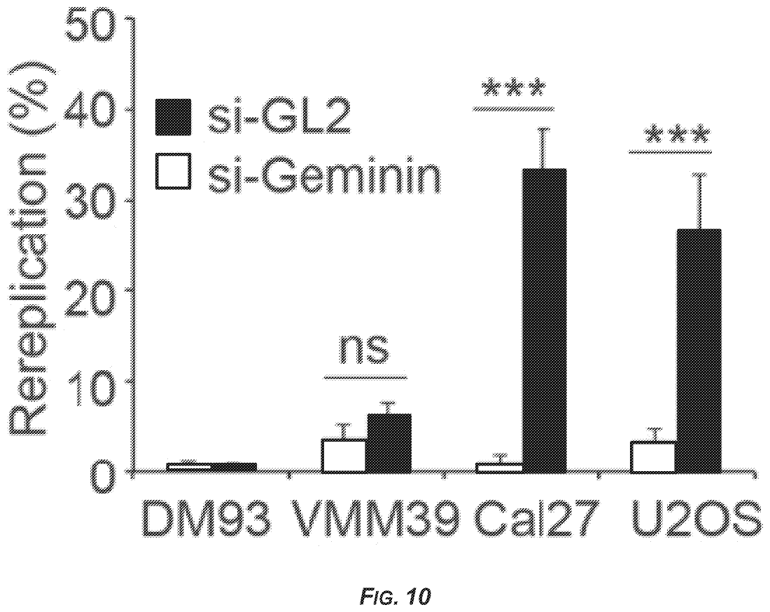

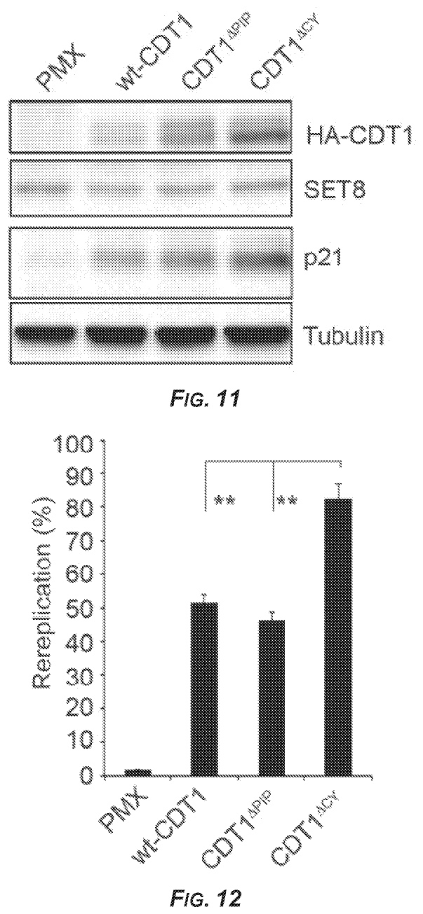

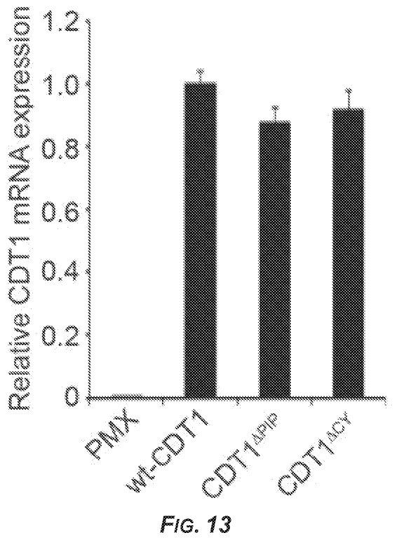

Thus, cyclin A, a cofactor required for CDT ubiquitylation via the SCF^{SKP2} ubiquitin ligase in S-phase (Abbas &Dutta, 2011; Abbas et al., 2013) appeared to restrain CDT1 activity in melanoma cells. Consistent with this hypothesis, overexpression of a SCF^{SKP2}-resistant mutant of CDT1 (CDT1^{ΔCY}), but not a CRL4^{CDT2}-resistant mutant (CDT1^{ΔPIP}; Senga al., 2006) in DM93 was more stable and induced rereplication more robustly than wild-type CDT1 (FIGS. 11-13, and Table 5). Thus, in melanoma cells, the steady state level of CDT1 was regulated primarily by cyclin A-mediated and CRL1^{SKP2}-dependent pathway.

TABLE 5
Rereplication in DM93 and VMM39 Melanoma
Cell Lines Overexpressing CDT1

| CDT1 Species | Percent Rereplication |
|---|---|
| PMX (negative control) | 1.74 |
| Wild-type CDT1 | 51.4 |
| CDT1^{□PIP} | 46.2 |
| CDTl^{□CY} | 82.6 |

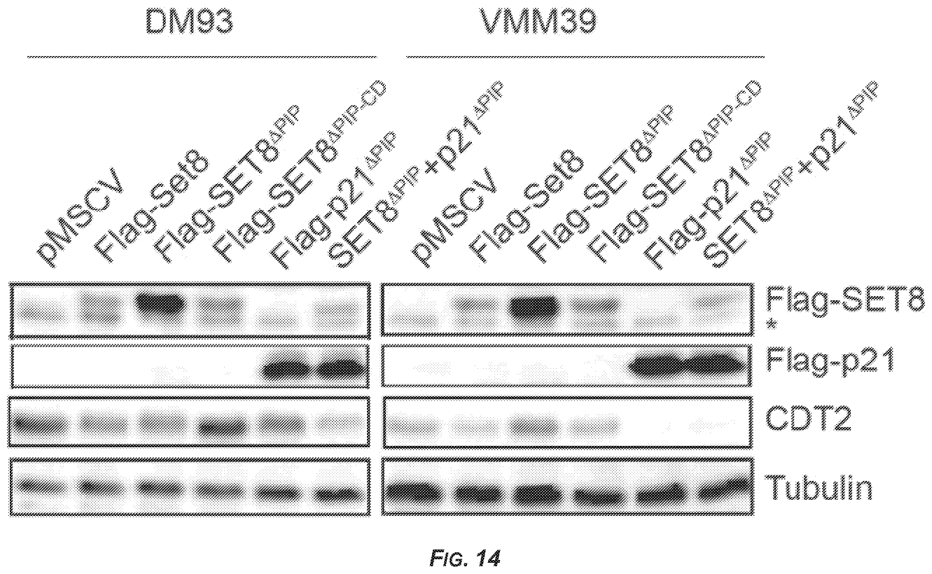

Unlike the case for CDT1, the stable overexpression of wild-type SET8 (or p21) in DM93 or VMM39 cells did not induce rereplication (FIGS. 14 and 15. and Table 6).

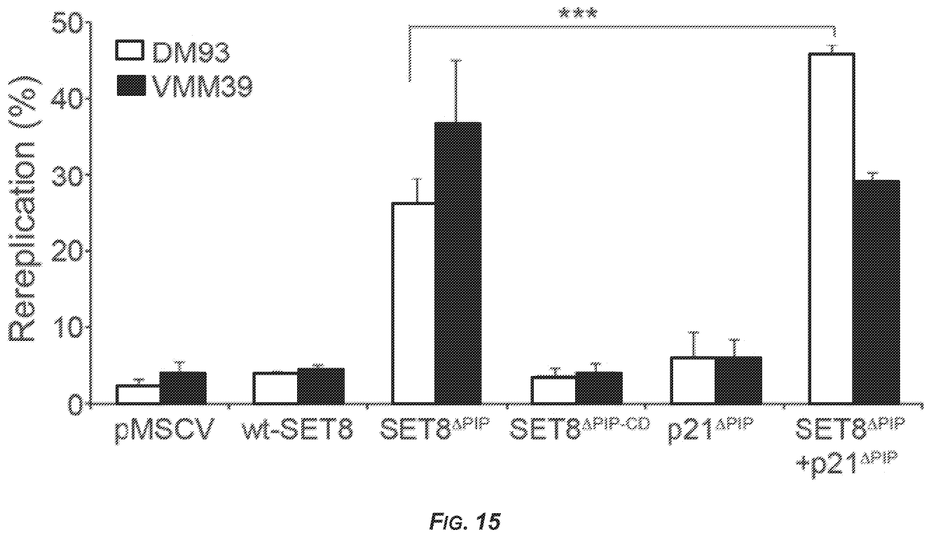

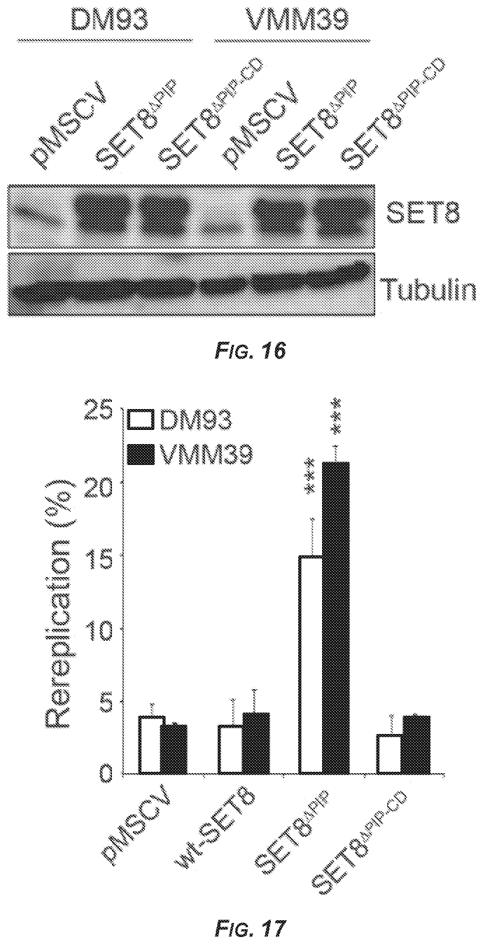

However, expression of SETS mutant protein that could not associate with PCNA (SET8^{ΔPIP}) and was thus resistant to CRL4^{CDT2} degradation (Abbas et al., 2010) induced rereplication in both lines, and this required SET8 catalytic activity (see FIGS. 14-17, and Table 6). It was noted that the catalytically inactive mutant of SET8^{ΔPIP} (SET8^{ΔPIP-CD}) was less stable than the catalytically active protein (see FIG. 14), and its overexpression from a higher titer virus relative to catalytically active SET8^{ΔPIP} did not induce rereplication either (FIGS. 16 and 17). Ectopic expression of CRL4^{Cdt2}-resistant p21 protein (p21^{ΔPIP}; Abbas et al., 2008) on the other hand, was associated primarily with growth arrest in early- and mid S-phase (FIGS. 14 and 15). Simultaneous expression of SET8^{ΔPIP} and p21^{ΔPIP} caused more rereplication in DM93 (˜46% compared to ˜27% with SET8^{ΔPIP} alone), but not in VMM39 cells (FIGS. 14 and 15). The lack of additive effects in VMM39 cells can be explained by the robust intra-S-phase cell cycle arrest caused by p21^{ΔPIP} expression in this line, preventing further rereplication.

TABLE 6
Rereplication in DM93 and VMM39 Melanoma
Cell Lines Overexpressing SET8 and/or p21

| | Percent | Percent |
|---|---|---|
| | Rereplication | Rereplication |
| SET8 and/or p21 Species | in DM93 | in VMM39 |
| pMSCV (negative control) | 1.24 | 4.13 |
| Wild-type SET8 | 3.87 | 4.98 |
| SET8^{ΔPIP} | 35.8 | 45.8 |
| SET8^{ΔPIP-CD} | 2.82 | 5.22 |
| P21^{ΔPIP} | 10.3 | 9.45 |
| SET8^{ΔPIP} + P21^{ΔPIP} | 46.4 | 13 |

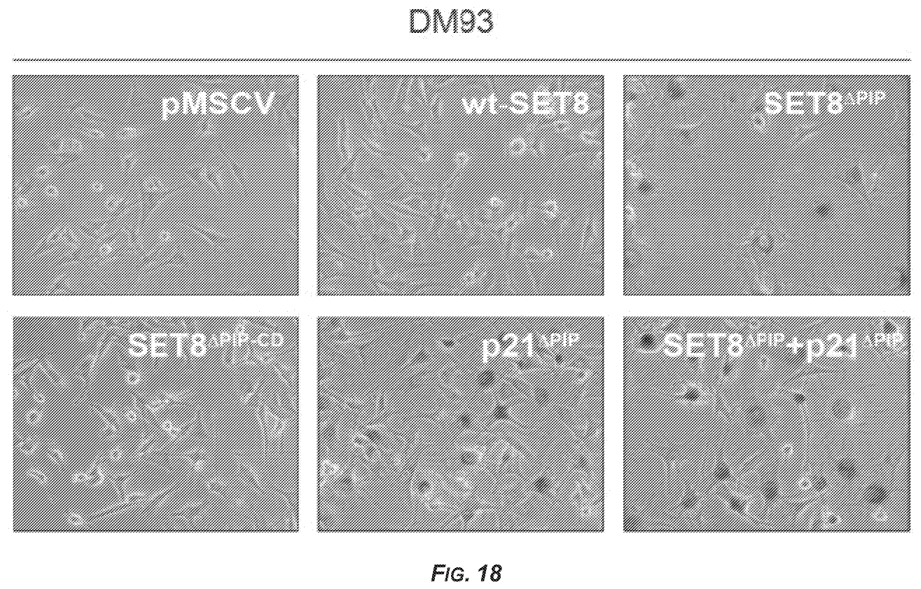

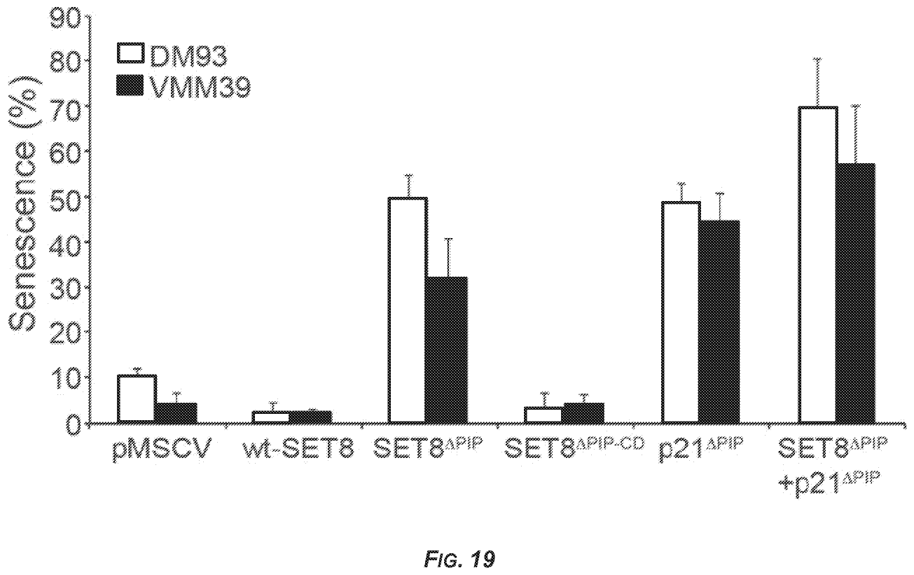

Finally, the expression of catalytically active SET8^{ΔPIP} or p21^{ΔPIP} induced senescence in both melanoma cell lines (FIGS. 18 and 19). Thus, deregulated SET8 expression appeared to be both required and sufficient to promote rereplication and senescence in CDT2-depleted melanoma cells.

### Example 4

### Pevonedistat Inhibited Melanoma Cells Through the Induction of Rereplication and Senescence, and Elevated CDT2 Expression Rendered Melanoma Cells Susceptible to Pevonedistat-Induced Rereplication

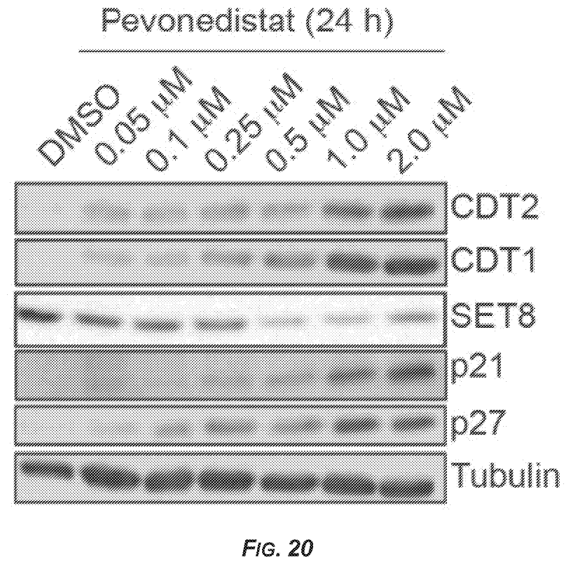

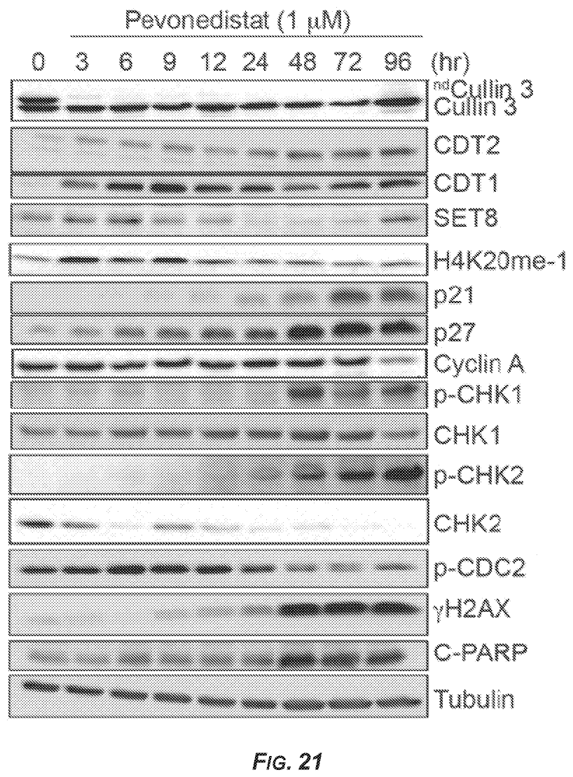

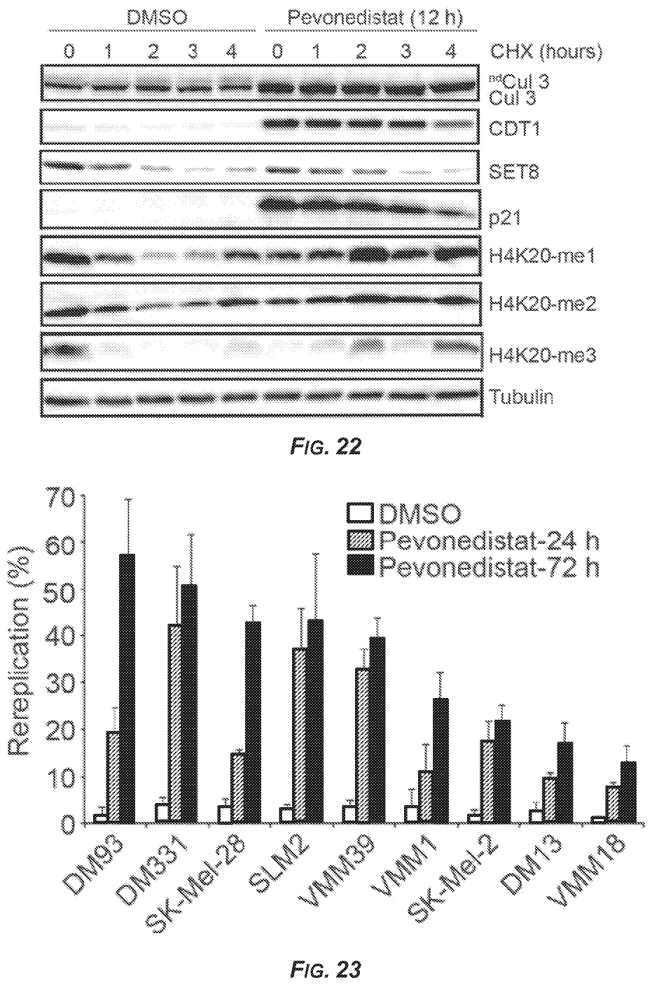

The CRL4^{CDT2} ligase, similar to all cullin-based ligases, is regulated by NEDD8 modification, which is catalyzed by an enzyme cascade system similar to ubiquitylation (Merlet et al., 2009). Pevonedistat inhibits cullin signaling, offering a pharmacological approach for targeting melanoma potentially through deregulated activity of the CRL4^{CDT2} ligase. To test this possibility, treated DM93 cells were treated with increasing doses of pevonedistat for 24 hours. This resulted in a dose-dependent increase in several cullin ubiquitylation substrates, including CDT2, CDT1, p21, and p27, which reached significant levels at 1 μM drug concentration (FIG. 20). Although CDT2 was increased by pevonedistat, it was likely to be inactive because of the de-neddylation of cullin proteins (FIGS. 20 and 21). Time course analysis with 1 μM pevonedistat demonstrated early (at 3 and 6 hours) increase in CDT1 as well as SET8 protein and activity (H4K20 mono-methylation) followed by the appearance of p21 and p27 (FIG. 21). Increases in CDT1, SET8, and p21 were all attributed to increased stability as well as increase in the stability of not only H₄K20me1, but also H₄K20me2 and H₄K20me3 (FIG. 22), which were upregulated in cells with deregulated SET8 stability and contribute to DNA rereplication (Abbas et al., 2010; Beck et al., 2012).

Within 24 hours of pevonedistat treatment (1 μM), DM93 cells accumulated spontaneous DNA damage (increased γH2AX) and arrested in S and G2/M phases of the cell cycle due to activated DNA damage and G2/M checkpoints as evidenced by increased phosphorylation of CHK1 and CHK2, increased phosphorylation of CDK1, and the accumulation of cells in S and G2 (Table 7; see also FIG. 21). This inhibited melanoma cell proliferation, concurrently with morphological changes reminiscent of those induced by CDT2 depletion or deletion. FACS analysis showed 18% of DM93 cells exhibiting >4N DNA content within 24 hours, which increased to 68% by 72 hours of treatment. The extent of rereplication increased with increasing doses of pevonedistat when analyzed at 24 hours, and was observed with as low as 500 nM drug concentration (see Table 8). BrdU labeling illustrated that rereplication occurred within the same cell cycle in 17.3% and 61.7% of cells when analyzed at 24 and 48 hours, respectively. Treatment of non-malignant melanocytes PIG3V with pevonedistat did not induce significant rereplication (less than 3%) when examined at 48 hours following treatment. While some DM93 cells treated with pevonedistat underwent cell death by apoptosis (i.e. appearance of cleaved PARP protein and increased cells with sub-G1 DNA content (˜7% at 72 hours)), the majority of cells underwent senescence occurring as early as 48 hours post-treatment.

TABLE 7
Increase in DM93 Cells in S and G2/M
Phases After Exposure to Pevonedistat

Percent Cells in

| Time | sub-G1 | S and G2/M |
|---|---|---|
| DMSO (negative control) | 0.99 | 1.8 |

| | | | |
|---|---|---|---|
| 3 | hours | 1.94 | 4.05 |
| 6 | hours | 0.49 | 13 |
| 24 | hours | 1.4 | 17 |
| 48 | hours | 2.08 | 64.7 |
| 72 | hours | 7.08 | 68.3 |

TABLE 8
Rereplication in DM93 Cells Increased
with Increased Pevonedistat at 24 Hours

Percent of Cells in

| [Pevondestat] (μM) | sub-G1 | S and G2/M |
|---|---|---|
| DMSO (negative control) | 1.39 | 0.7 |
| 0.025 | 1.72 | 0.57 |
| 0.500 | 1.61 | 1.11 |
| 0.100 | 5.62 | 2.21 |
| 0.500 | 4.49 | 18.1 |
| 0.750 | 2.05 | 47.4 |
| 1.000 | 1.21 | 56.6 |
| 2.000 | 0.53 | 66 |

Using clonogenic survival assays, it was determined that pevonedistat inhibited the proliferation of DM93 and VMM39 at low drug concentrations of 100 and 50 nM, respectively (see Table 9). Using standard cell proliferation/viability MTT assays, the sensitivity of a panel of melanoma cells to pevonedistat was determined. The results demonstrated that pevonedistat effectively inhibited all the melanoma lines tested, with VMM39 and DM13 being the most sensitive with IC₅₀ of 35 and 40 nM, respectively. On the other hand, VMM1 melanoma cells were least sensitive with an IC₅₀ of 330 nM. Pevonedistat resulted in varying degrees of rereplication and apoptosis in these cells (FIG. 23).

TABLE 9
Rereplication in Various Cell Lines Treated
with Pevonedistat at 24 and 72 Hours

| | Percent of Cells in S and G2/M at | | |
|---|---|---|---|
| Cell Line | DMSO (Control) | 24 hours | 72 Hours |
| DM93 | 1.61 | 27.2 | 59.2 |
| DM13 | 2.06 | 10.4 | 13.7 |
| VMM18 | 1.1 | 7.83 | 16.9 |
| VMM39 | 1.71 | 37.9 | 33.8 |
| SLM2 | 2.63 | 46.8 | 50.2 |
| SK-MEL-28 | 2.64 | 38.7 | 55.9 |
| DM331 | 1.2 | 47.8 | 63.2 |
| VMM1 | 1.6 | 7.72 | 30.8 |

For example, whereas pevonedistat treatment of DM93 cells resulted in 60% of the cells undergoing rereplication at 72 hours post-treatment, only 25% of VMM1 and 13.7% of DM13 cells rereplicated their DNA. SLM2 melanoma cells with wild-type BRAF, NRAS, TP53, and CDKN2A also exhibited robust rereplication with more than 58% of cells with rereplication at 72 hours post-treatment. Although CDT2 expression in the various lines did not correlate significantly with the IC₅₀ of pevonedistat, it significantly correlated with pevonedistat-induced rereplication (r=0.745, p<0.01), demonstrating that pevonedistat might exhibit inhibitory activity in addition to its ability to induce rereplication (see below).

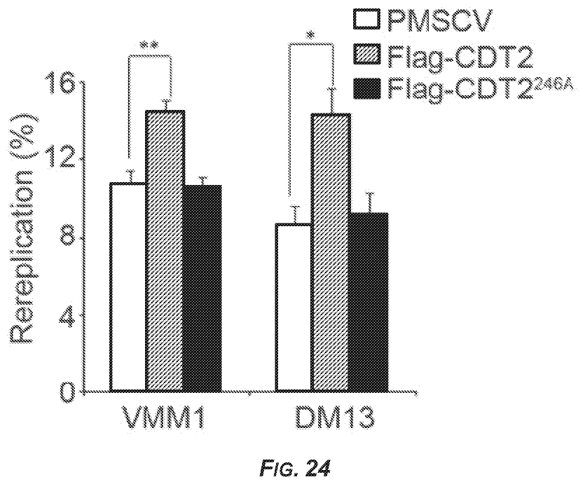

Strikingly, it was determined that overexpression of wild-type CDT2 in two melanoma cell lines with low expression of CDT2 (VMM1 and DM13) resulted in the induction of statistically significantly more rereplication in response to pevonedistat than control cells with empty virus (pMSCV; p<0.01 for VMM1 and p<0.05 for DM13, calculated using Student's t-test; see FIG. 24). This did not occur with overexpression of a mutant CDT2 protein (CDT2R^{246A}), which could not bind DDB1 (Jin et al., 2006) and was thus incapable of assembling functioning CRL4^{CDT2} ligase. This result provided evidence that CDT2 expression directly related to the efficacy of pevonedistat to induce rereplication in vitro through its ubiquitylation activity.

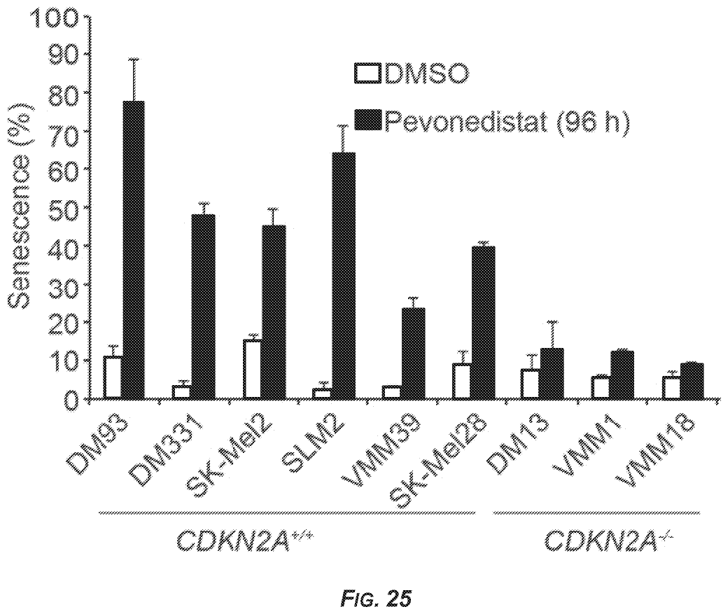

Finally, although pevonedistat induced rereplication in all the examined melanoma lines, it induced robust senescence only in cells with wild-type CDKN2A (encoding p16), with minimal impact in cells with an inactivated CDKN2A gene (VMM1, DM13, and VMM18 cells), similar to what was observed in si-CDT2 cells (FIGS. 3 and 25). Collectively, these results demonstrated that pevonedistat treatment of melanoma cells was associated with the hallmark of CRL4^{CDT2} inactivation observed with CDT2 depletion or deletion. The results also demonstrated the efficacy of pevonedistat to inhibit melanoma in vitro, irrespective of the BRAF/NRAS or CDKN2A mutational status, although the latter could be important for pevonedistat-induced senescence.

### Example 5

### Transient Exposure to Pevonedistat was Sufficient to Induce Rereplication and Permanent Growth Arrest in Melanoma Cells, but not in Immortalized Non-Transformed Melanocytes

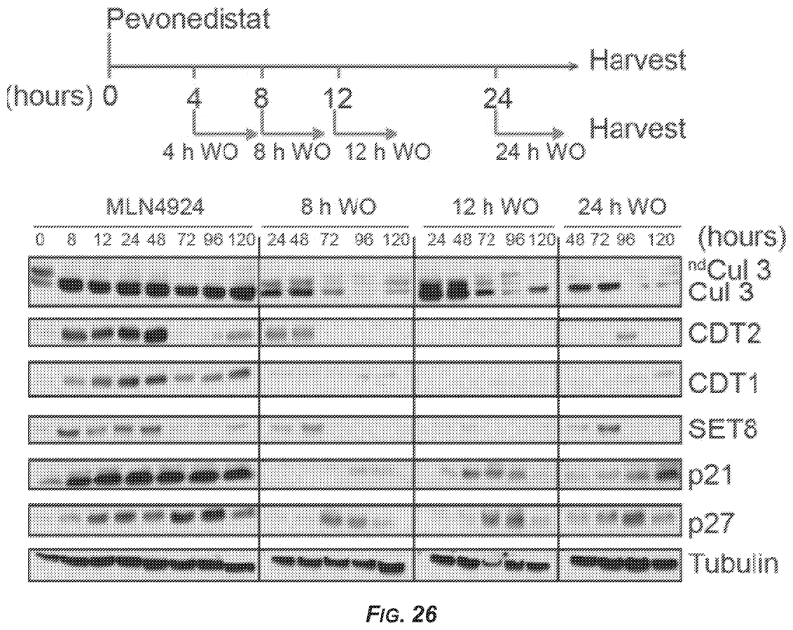

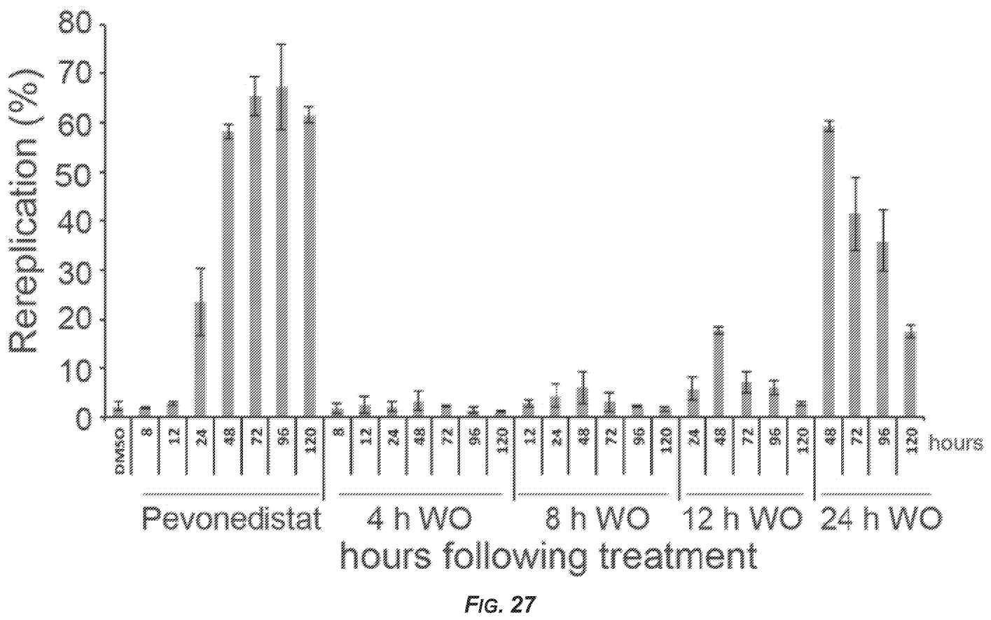

To determine the relationship between the ability of pevonedistat to induce rereplication in melanoma cells and its inhibitory activity, DM93 or VMM39 cells were treated with pevonedistat for 4, 8, 12, or 24 hours, washed extensively, and incubated with fresh media for various time points. The results demonstrated that cells exposed to pevonedistat for 4 or 8 hours were transiently inhibited, but resumed proliferation 24 hours later (FIGS. 26 and 27). Resumption of cell proliferation coincided with restoration of cullin neddylation and the destabilization of CDT1, SET8, and p21 (FIG. 26). In contrast, cells treated with pevonedistat for 12 or 24 hours remained arrested, exhibited 18% and 60% rereplication, respectively, when analyzed 48 hours later, and were senescent (FIGS. 26 and 27). The reduction in the percentage of rereplicating cells at 48, 72, and 92 hours following transient exposure for 12 or 24 hours can be explained by the continuous proliferation of cells that did not undergo rereplication following drug removal. DM93 cells treated for 12 or 24 hours maintained high levels of p21, but not CDT1 or SET8 (FIG. 26), suggesting that p21 might be essential for maintaining the rereplication phenotype.

Furthermore, treatment of DM93 cells with the BRAF-kinase inhibitor vemurafenib (PLX4032) induced robust G1 growth arrest and complete depletion of S-phase cells, and inhibited pevonedistat-induced rereplication. FACS analysis demonstrated that DM93 cells treated with 1 micromolar (1 μM) pevonedistat for 48 hours underwent significant rereplication (45%) and pretreatment with vemurafenib for 24 hours reduced the percent of cells undergoing rereplication to 16%. This result, and the fact that it takes at least 24 hours to achieve permanent growth inhibition, demonstrated that pevonedistat-induced rereplication required that cells remain in active replicative phase, and that a sufficient time of exposure (12-24 hours) was necessary to permanently arrest all cycling cells.

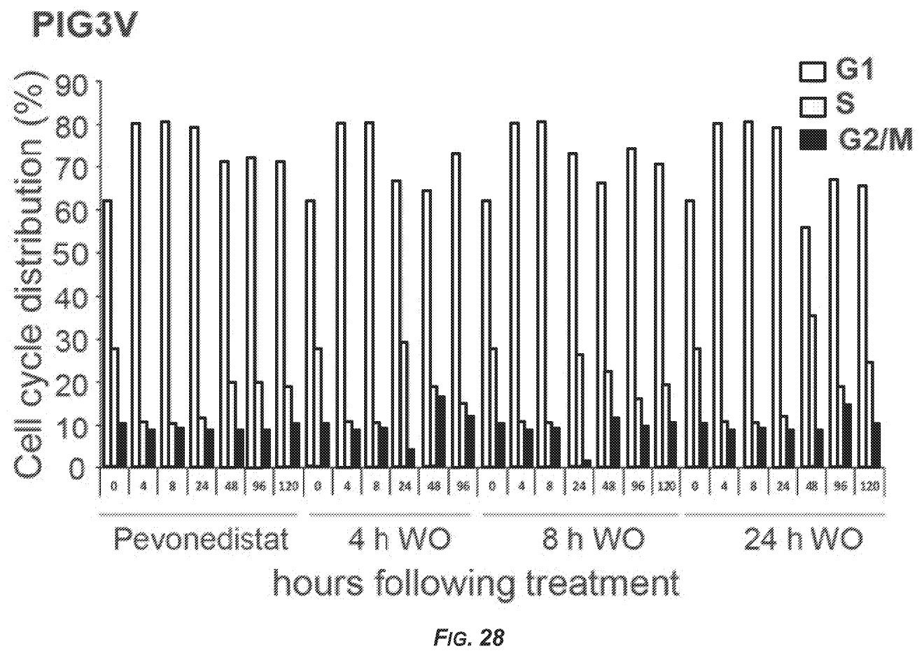

Unlike melanoma cells, the treatment of PIG3V or PIG1 immortalized but not malignant melanocytic cell lines with pevonedistat resulted in only modest inhibition of proliferation with an IC₅₀ of >500 nM. Although the continuous treatment of PIG3V with 1 μM pevonedistat inhibited proliferation (FIG. 28), this was not associated with rereplication or senescence. This was not due to a lack of inhibition of CRL4^{CDT2}, as these cells accumulated the CRL4^{CDT2} substrates CDT1, p21, and SET8 (which were only transiently upregulated) as well as other cullin substrates, such as p27, with similar kinetics as in DM93 (FIGS. 26 and 29). PIG3V melanocytes exposed to 1 μM pevonedistat for 24 hours arrested in G1, but resumed cycling following drug removal, and this was associated with the reversal of cullin neddylation and CRL4^{Cdt2} substrate accumulation, including p21 (FIGS. 28 and 29).

### Example 6

### Pevonedistat Induced Permanent Growth Inhibition in Melanoma Cells Through SETS- and p21-Dependent Rereplication and Senescence

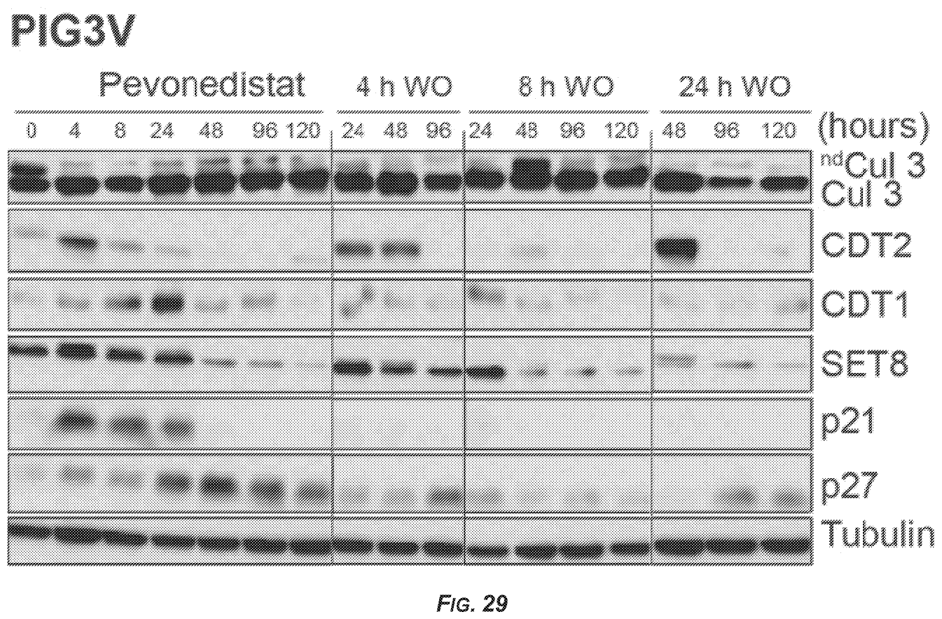

The lack of significant correlation between pevonedistat-induced rereplication and toxicity prompted an investigation into the contribution of rereplication and/or senescence to pevonedistat-induced toxicity. To achieve this, it was first shown that siRNA-mediated depletion of CDT1, p21, or SET8 all inhibited pevonedistat-induced rereplication, and depletion of CDT1 or p21, and to a lesser extent SET8, inhibited pevonedistat-induced senescence, similar to what was observed following CDT2 depletion (FIGS. 30 and 31).

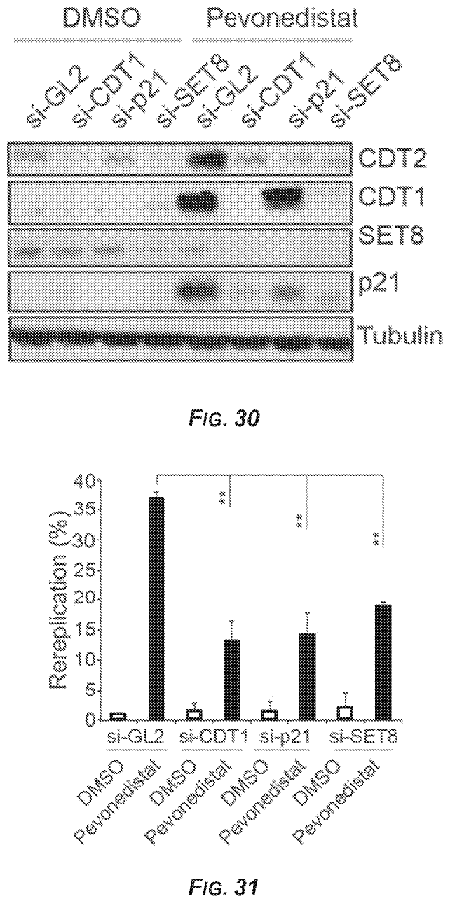

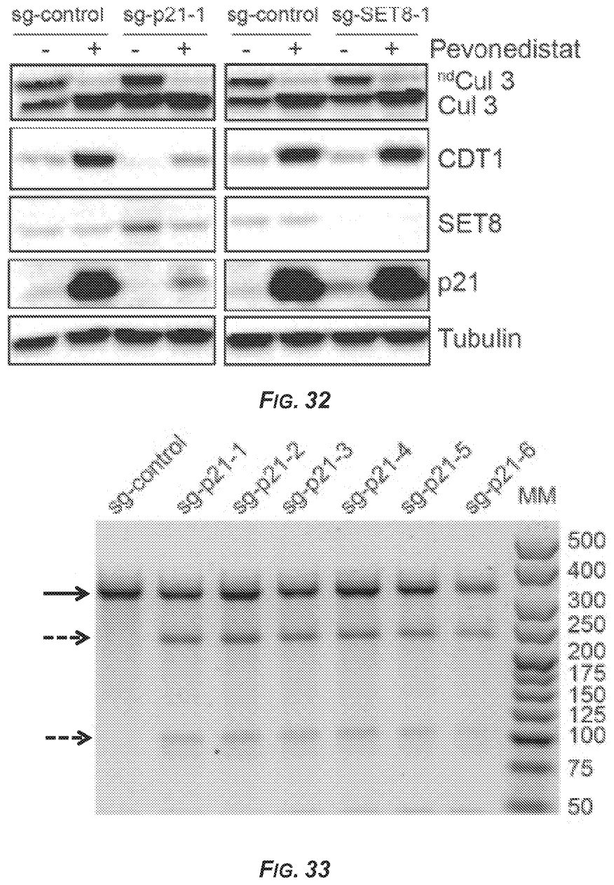

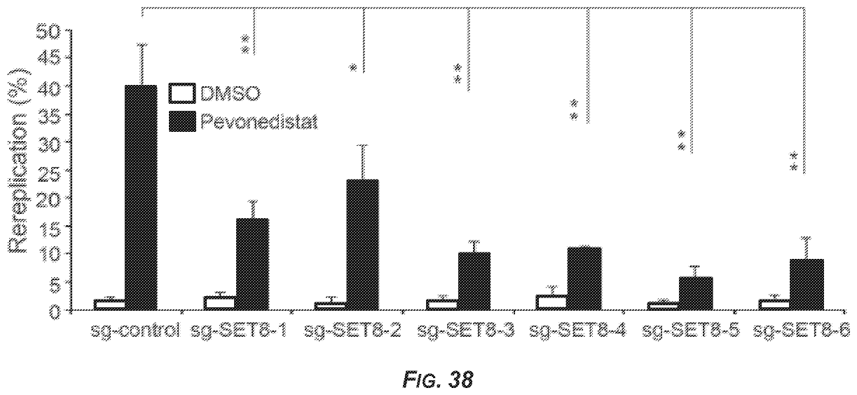

Next, the CRISPR/Cas9 editing tools were employed in an attempt to generate melanoma cells with deletion in CDKN1A (encoding p21) or SET8. Several clones of DM93 cells were obtained, but none had bi-allelic deletion of either gene. Although it was surprising that none of the clones obtained were biallelically deleted of CDKN1A, the lack of clones with complete deletion of SET8 was consistent with the important role of SET8 in cell viability (Oda et al., 2010; Schotta et al., 2008). Nevertheless, several individual clones exhibited a loss of one allele of CDKN1A (sg-p21-1-6), and these exhibited significantly reduced levels of p21 in pevonedistat-treated cells (FIGS. 32, 33, 35, and 37).

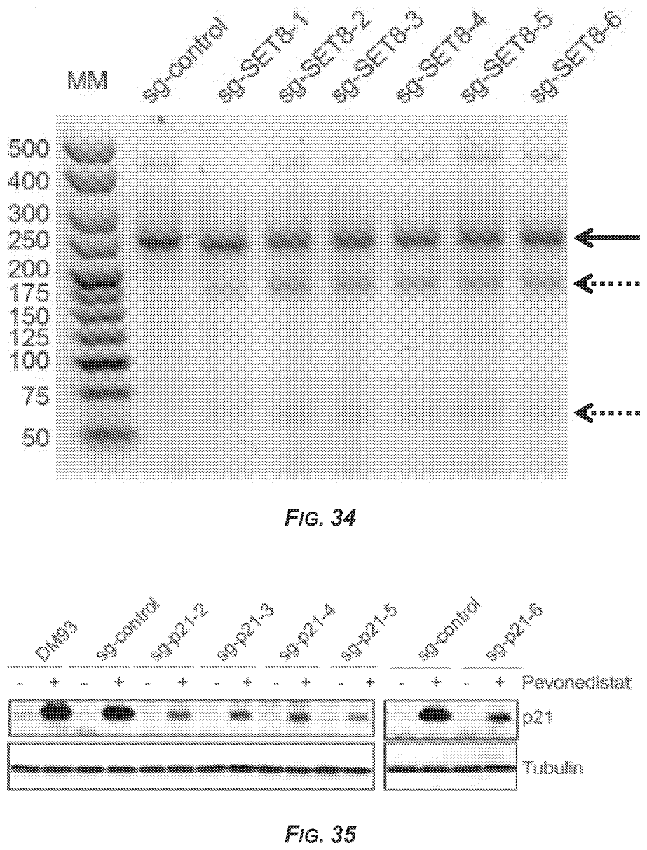

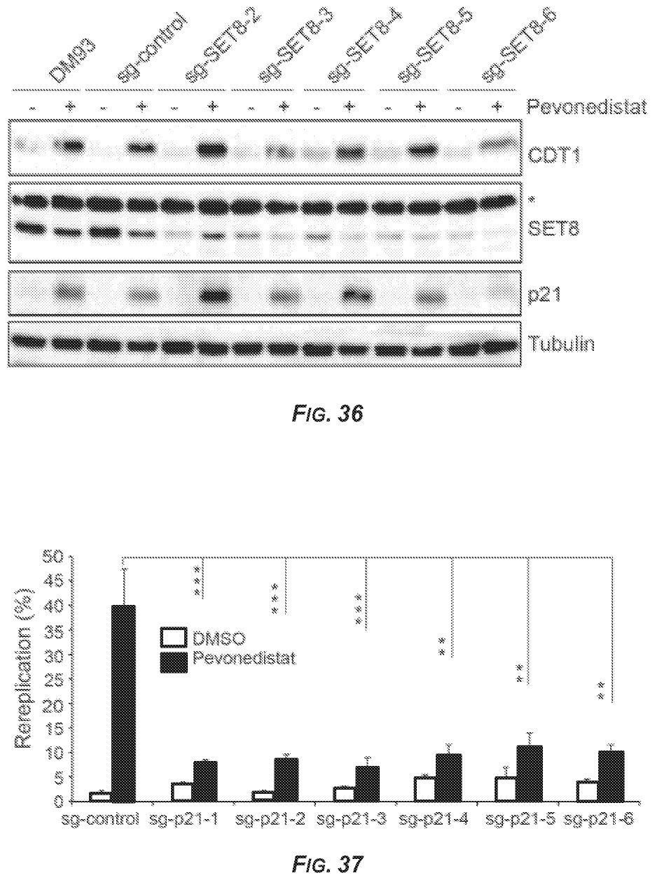

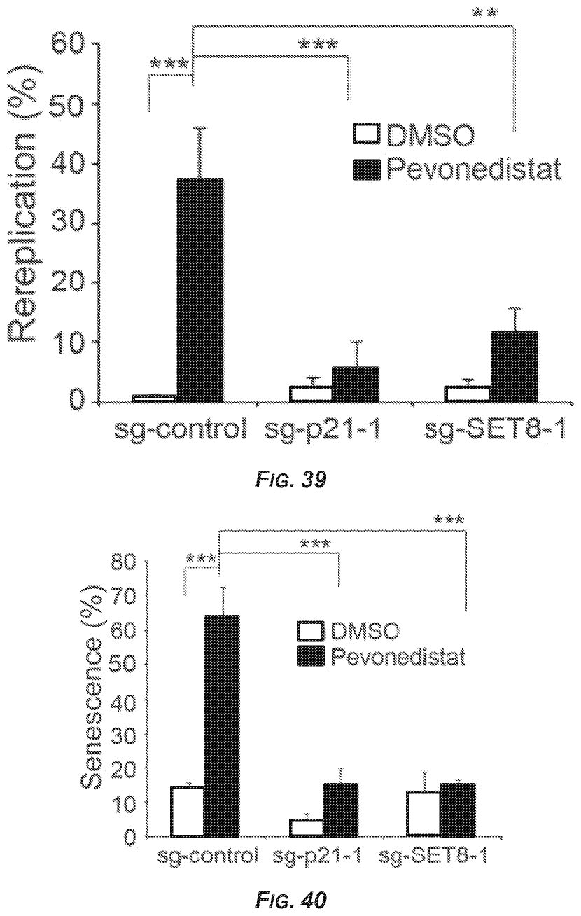

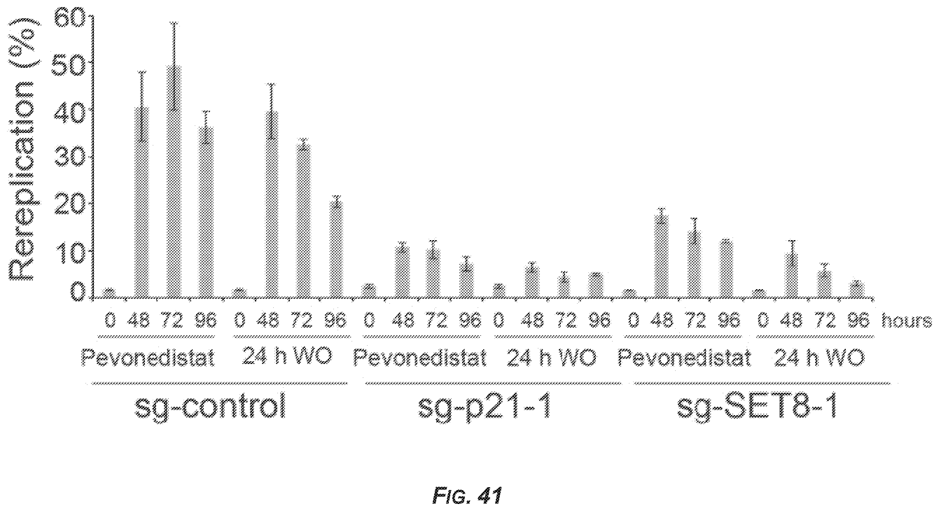

Also obtained were several clones of DM93 cells with monoallelically deleted SET8 (sg-SE78-1-6), and these had significantly reduced levels of SET8 protein (FIGS. 32, 34, 36, and 38). The sg-SET8 cells exhibited normal levels of bulk H4K20me1 and proliferated with similar rates as parental or control DM93 cells. Importantly, both p21 and SET8 hypomorphic DM93 cells were significantly resistant to pevonedistat-induced rereplication, despite cullin deneddylation, and the upregulation of CDT1 protein (FIGS. 32, 35, and 38-40). This result demonstrated that increased endogenous levels of CDT1 was not sufficient to induce rereplication or senescence in the presence of lower levels of SET8 or p21, and further suggested that pevonedistat-induced senescence in melanoma cells was a consequence of DNA rereplication. Given that melanoma cells with higher levels of CDT2 were more susceptible to pevonedistat-induced rereplication (FIGS. 22 and 23), and only the overexpression of CRL4^{CDT2}-resistant, but not CRL4^{CDT2}-sensitive form of SET8 was sufficient to trigger rereplication (FIG. 15), the failure of pevonedistat to induce rereplication and senescence in sg-p21 or sg-SET8 cells highly suggested that pevonedistat-induced rereplication and senescence in melanoma cells was mediated through CRL4^{CDT2} inhibition and the stabilization of SET8 and p21 proteins.

When added continuously in culture, pevonedistat inhibited the proliferation of sg-p21 and sg-SET8 cells. Strikingly, however, unlike control cells (sg-control), cells with reduced expression of p21 or SET8 resumed proliferation following the cessation of pevonedistat treatment (FIG. 41). Thus, pevonedistat inhibited the proliferation of melanoma cells through the induction of SET8- and p21-dependent rereplication mechanism, as well as through another mechanism that only transiently inhibited cell proliferation. The result also explains the lack of a significant correlation between pevonedistat-inhibitory activity (IC₅₀) and the induction of rereplication.

### Example 7

### Pevonedistat Exerted Anti-Melanoma Activity Through CRL4^{CDT2} Inhibition and Stabilization of SET8 and p21, Irrespective of BRAF/NRAS Mutational Status

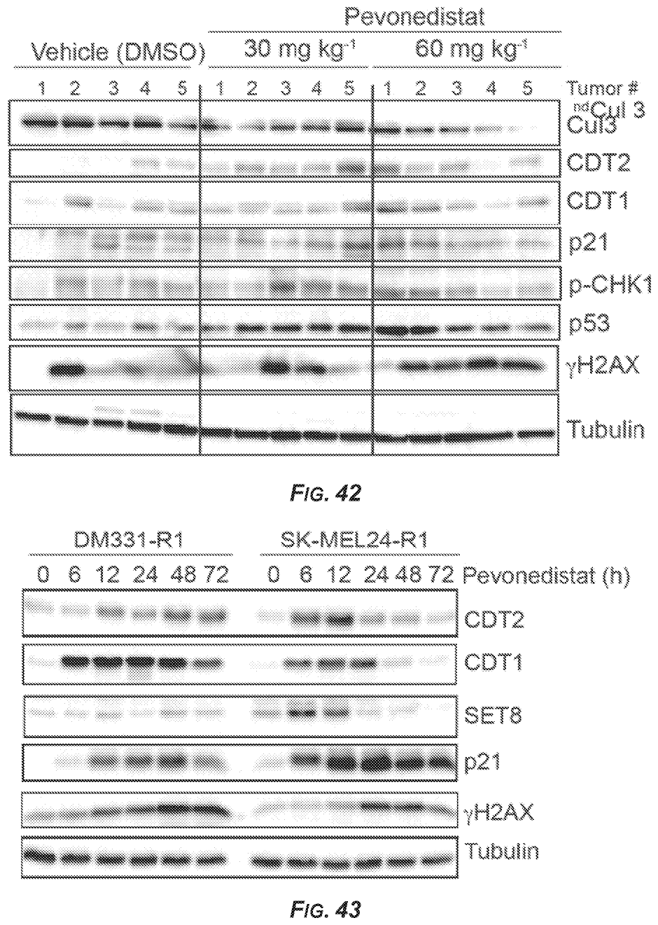

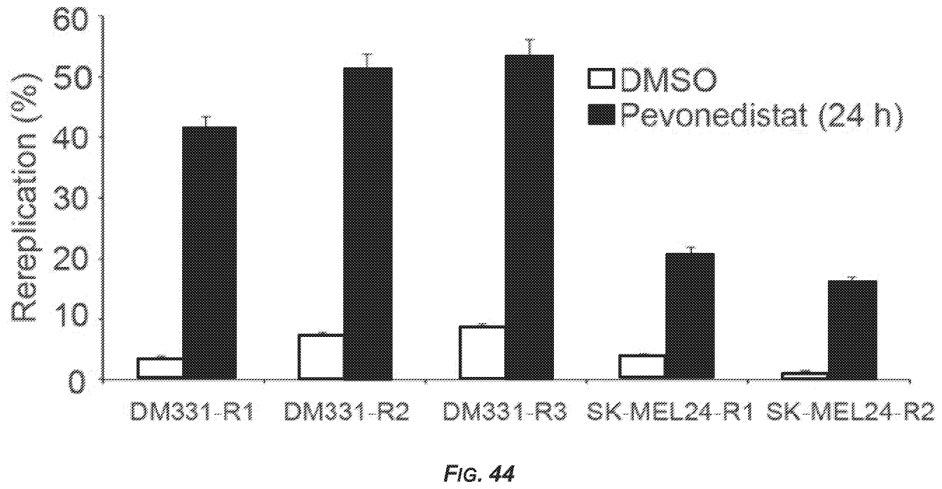

To examine the efficacy of pevonedistat to suppress melanoma in vivo, nude mice were inoculated with DM93 cells and tumor growth was monitored. When tumors reached 100-150 mm³ in volume, randomized group of mice (n=12) were treated with DMSO or with 30 or 60 mg/kg for five (5) consecutive days for two cycles separated by five (5) days of no treatment (Soucy et al., 2009). Animals were weighed and monitored daily and the drug was well tolerated. Pevonedistat significantly suppressed DM93 melanoma xenografts at 30 mg/kg (p=7.7×10³) or 60 mg/kg (p=2.3×10³), but did not result in tumor regression, consistent with the lack of significant apoptotic effect of this drug in vitro. Tumor regrowth was not detectable at either drug concentration as monitored up to 10 days following the cessation of treatment. Analysis of tumor extracts of treated animals (on day 25) demonstrated that pevonedistat inhibited cullin neddylation and resulted in the accumulation of cullin substrates (CDT2, CDT1, and p21) and spontaneous DNA damage, and exhibited activated checkpoints (FIG. 42). Because SET8 protein was only transiently increased by pevonedistat, significant increases of SET8 were not detected in these tumor lysates at this late time point. Importantly, although pevonedistat (30 mg/kg) significantly inhibited the growth of sg-control DM93 xenografts (p=0.009), it failed to inhibit the growth of sg-p21-1 or sg-SET8-1 DM93 xenografts (p=0.092 and 0.66, respectively). This result provided evidence that targeted inactivation of the CRL4^{CD}-2 E3 ligase, and the stabilization of its substrates p21 and SET8, was the primary mechanism underlying the anti-melanoma activity of pevonedistat in vivo.

To test whether pevonedistat equally suppressed non-BRAF melanomas, xenografts of the VMM39 (with NRAS and PDGFR activating mutations) or with SLM2 cells (without NRAS or BRAF mutations) were established. Pevonedistat (60 mg/kg) significantly inhibited the growth of VMM39 and SLM2 xenografts (p=1.8×10⁻⁵, and 2.3×10⁻³, respectively), although tumor regrowth was apparent in these xenografts following the cessation of drug administration. Similar to DM93 xenografts, pevonedistat inhibited the deneddylation of cullins and induced the accumulation of CDT1 and p21 proteins in the VMM39 xenografts, even when tumors analyzed 10 days following the cessation of treatment.

Pevonedistat also inhibited (p<0.01 vs. vehicle, calculated using Student's t-test), albeit to a lesser extent, the growth of DM331 xenografts, a mutant BRAF melanoma cell line resistant to the BRAF kinase inhibitors vemurafenib and PLX4720, a structural analogue and precursor of vemurafenib with more potent activity in rodents (Tsai et al., 2008; see also Roller et al., 2015). Although these xenografts were nevertheless inhibited by PLX4720 (p<0.001 vs. vehicle, calculated using Student's t-test), the combined administration of pevonedistat and PLX4720 resulted in synergistic inhibition (p<0.001 vs. vehicle, calculated using Student's 1-test).

DM331 cells as well as the BRAF-mutant SK-MEL-24 cells extracted ex vivo from PLX4720-resistant tumors (Roller et al., 2015) remained insensitive to vemurafenib (PLX4032), but were sensitive to pevonedistat-induced rereplication and inhibition in vitro (FIGS. 43 and 44). Collectively, these results demonstrated that the administration of pevonedistat as a single agent robustly suppressed melanoma in vivo, irrespective of the BRAF/NRAS mutational status, synergized with vemurafenib to suppress BRAF melanoma, and effectively inhibited vemurafenib-relapsed melanoma cell growth.

### Materials and Methods for Examples 8-13

Tissue Culture and Reagents.

Cal27, FaDu, and SCC25 HNSCC cells were obtained from the American Type Culture Collection (ATCC, Manassas, Va., United States of America). UNC7 cells were provided by Dr. Wendell Yarbrough (Vanderbilt University, Nashville, Tenn., United States of America). Cells were grown in Dulbecco's modified Eagle's medium/Ham's nutrient mixture F12 supplemented with 10% fetal bovine serum (FBS) and 1% penicillin/streptomycin. OKF6-TERT2 cells were purchased from Dr. James Rhienwald at Harvard Medical School (Department of Dermatology, Boston, Mass., United States of America) and were cultured in GIBCO® brand keratinocyte serum-free medium (K-sfm), supplemented with 25 μg/ml Bovine Pituitary Extract (BPE), 0.4 mM CaCl₂), 0.2 ng/ml epidermal growth factor (EGF), and 1% penicillin/streptomycin. All cell lines were grown at 37° C. in 5% CO₂. MLN4924 (pevonedistat) was purchased from Active Biochem (Wan Chai, Hong Kong), and was dissolved in 10% DMSO in sterile PBS. Propidium iodide, 7-AAD, and BrdU kit were purchased from BD Biosciences (San Diego, Calif., United States of America). Antibodies against p21 (C19), p53 (DO-1), geminin (FL-209), and actin (I-19) were purchased from Santa Cruz Biotechnology, Inc. (California, United States of America). Antibodies against SET8, CHK1, CHK2, γH2AX, p-CHK1 (S375), p-CHK2 (T68), p-p53 (S15), and PARP were purchased from Cell Signaling Technology, Inc. (Danvers, Mass., United States of America). Anti-Cul3 antibody was purchased from Bethyl Laboratories (Montgomery, Tex., United States of America). Anti-EMI1 antibody was purchased from Life Technologies, Inc., (Carlsbad, Calif., United States of America). Anti-CDT1 and anti-CDT2 antibodies were described before (Abbas et al., 2010).

Cell Lysis, SDS-PAGE and Immunoblotting.

HNSCC cells were lysed using radio-immunoprecipitation assay (RIPA) lysis buffer (50 mM Tris, pH 8.0; 150 mM NaCl, 1% NP-40; 0.5% sodium deoxycolate; 0.1% SDS; 1 mM Benzamidin-HCl; 0.5 μg/ml Leupeptin; 0.5 μg/ml Aprotinin; 1 μg/ml pepstatin; 20 mM NaF; 20 mM Na₃VO₄). Equal amounts of protein were electrophoretically separated in a polyacrylamide 8-12% gel (Bio-Rad Laboratories, Inc., Hercules, Calif., United States of America), trans-blotted to a nitrocellulose membrane, and incubated with primary antibodies for one hour at room temperature or overnight at 4° C. The immunoblot signals were detected by enhanced chemiluminescence (EMD Millipore Corporation, Billerica, Mass., United States of America).

Si-RNA-Mediated Gene Silencing.

Transfections of different si-RNAs (10 nM) were performed using LIPOFECTAMINE® RNAimax brand transfection reagent according to the manufacturer's protocol (INVITROGEN™, Carlsbad, Calif., United States of America). The following si-RNAs (sense strands) were used: si-GL2: 5′-AACGUACGCGGAAUACUUCGA-3′ (SEQ ID NO: 1); si-CDT2: 5′-GAAUUAUACUGCUUAUCGA-3′ (SEQ ID NO: 2); si-geminin: 5′-UGCCAACUCUGGAAUCAAA-3′ (SEQ ID NO: 6); si-EMI1-1: 5′-GAGAAUUUCGGUGACAGUCUA-3′ (SEQ ID NO: 7); and si-EMI1-2: 5′-UACGAAGUGUCUCUGUAAUUA-3′ (SEQ ID NO: 8).

Cell Proliferation and Clonogenic Survival Assays.

HNSCC cells were transfected with si-RNA (48 hours prior to first count) or treated with pevonedistat (24 hours prior to first count). 8×10⁵ cells were seeded in 60 mm plates and cell proliferation was determined by staining with trypan blue and counting by COUNTESS™ Automated Cell Counter (INVITROGEN™, Carlsbad, Calif., United States of America). Depending on the cell growth rate, cell counts were recorded either every 24 or 48 hours, and growth curves were established.

Clonogenic Survival Assays.

The effect of pevonedistat treatment or transient silencing of CDT2, geminin, or EMI1 on cell proliferation or on radiation sensitivity was tested using clonogenic survival assays. Cells were transfected with the appropriate si-RNA 48 hours prior to seeding. Cells were then counted using COUNTESS™ Automated Cell Counter (INVITROGEN™, Carlsbad, Calif., United States of America) and were seeded at 15,000 cells/plate in 60 mm dishes. Cells were irradiated 24 hours after seeding with various doses and were cultured for 7-10 days. Once colonies reached the appropriate size (>50 cells each), cells were washed twice with cold PBS, fixed in cold 100% methanol for 10 minutes, and stained with crystal violet (0.5%) for 10 minutes. The plates were washed, dried, and imaged using IMAGE LAB™ software (BioRad Laboratories, Inc., Hercules, Calif., United States of America). QUANTITY ONE® software (BioRad Laboratories, Inc., Hercules, Calif., United States of America) was used to quantify the number of colonies and survival curves were established based on the linear quadratic model, using the formula S=e^{-αD-βD2}; where S represents the surviving fraction and D the dose of irradiation. Results are represented as mean standard deviation (SD) of three independent experiments normalized to the corresponding non-irradiated plates for each group. When the effect of pevonedistat on cell radiosensitivity was tested, cells were seeded at 15,000 cells/60 mm plate and allowed to adhere for 4-6 hours. Pevonedistat was then added upon cell adherence at varying concentrations and cells were irradiated the following day. The duration of the experiment and the stopping procedure were as described above.

Flow Cytometry Analysis.

The effect of pevonedistat treatment or CDT2, geminin, or EMI1 knockdown on the cell cycle (and induction of DNA rereplication) was assessed by flow cytometry with propidium iodide (PI) staining. Cells were harvested at 72 or 96 hours post-treatment with pevonedistat or post-transfection, respectively. Cells were collected, washed with PBS, and resuspended in ethanol (75%). Cells were subsequently treated with 20 μg of DNase-free RNase and stained with PI following the manufacturer's protocol. FACSCAN™ (Becton, Dickinson and Company, Franklin Lakes, N.J., United States of America) was used to analyze the samples and G0-G1, S, and G2-M fractions were segmented. Subsequent analysis using FLOWJO® (FLOWJO, LLC, Ashland, Oreg., United States of America) and ModFit (Verity Software House, Topsham, Me., United States of America) software was used to determine apoptotic and re-replicating fractions. Where indicated, Cal27 and FaDu cells were treated with pevonedistat for 48 hours and pulsed with BrdU (10 nM) for 1 hour in the dark prior to harvesting. Cells were washed with PBS and staining solution before fixation and permeabilization steps according to the manufacturer's protocol. Cells were subsequently stained with anti-BrdU antibody solution for 20 minutes at room temperature, washed, and stained with 7-AAD for 30 minutes at 4° C. Cells were resuspended in 1 ml of staining buffer and stored at 4° C. overnight before analysis. Sampled were analyzed on a FACSCAN™ (Becton, Dickinson and Company, Franklin Lakes, N.J., United States of America), and different fractions of BrdU positive cells were determined using FLOWJO® (FLOWJO, LLC, Ashland, Oreg., United States of America) and ModFit (Verity Software House, Topsham, Me., United States of America) software.

In Vivo Xenograft Mice Experiments.

The animal studies were conducted in accordance with the guidelines established by the University of Virginia Animal Care and Use Committee (ACUC). The effect of pevonedistat on tumor growth was tested in a flank HNSCC xenograft model. 4-5 weeks old Foxn1^{nu} athymic female nude immunodeficient mice (20-25 g body weight; Harlan Laboratories, now Envigo, Indianapolis, Ind., United States of America) were used. Pevonedistat was prepared in 10% DMSO containing PBS and filtered before use. 5×10⁶ Cal27 cells (suspended in 200 μl sterile PBS) were inoculated subcutaneously in both flanks of nude mice (8 mice per group). When the tumor size reached 100 mm³ (10 days post-inoculation), mice were randomized and were treated with pevonedistat (20 mg/kg), or with control vehicle (DMSO), administered intraperitoneally on a regimen of 5 days on/5 days off for 2 cycles (Tardat et al., 2010). Tumors from a third group of mice were exposed to 1Gy irradiation (IR) daily, 5 days/week for 3 weeks, and a fourth group of mice received both pevonedistat and IR treatments. Tumor irradiation was performed at the University of Virginia X-Ray facility, and only the tumors on both flanks were irradiated while the rest of animal body was shielded. For combination treatment, pevonedistat was given 2 hours prior to radiation exposure with the same schedule as for the individual treatments. Tumor growth was monitored every other day using an electronic caliper, for 3 weeks post-treatment and average of tumor volumes were calculated using the formula (L×W²)/2). The results are represented as the mean tumor volumes±s.e.m, and p<0.05 was considered significant. Mice were weighed once a week during the entire course of the experiment and no significant effect of either treatment was observed.

Kaplan-Meier Plot Analysis.

The Cancer Genome Atlas (TCGA) data, publicly available at cBioPortal (Cerami et al., 2012; Gao et al., 2013), was used to plot Kaplan-Meier plots on tumors divided into two groups based on CDT2 expression as a Z-score (Taylor et al., 2010; Collisson et al., 2014; Weinstein et al., 2014).

Statistical Analysis.

All experiments were performed in triplicates and results with p values <0.05 were considered statistically significant. All quantitative differences were analyzed by Student's t-test. Synergy was determined using the Bliss model of independence (Bliss, 1939; Fitzgerald et al., 2006).

### Example 8

### CDT2 is Overexpressed in Head and Neck Squamous Cell Carcinoma

CDT2 expression is elevated in a number of human malignancies including breast, gastric, liver, brain, and skin cancers (Pan et al., 2006; Ueki et al., 2008; Li et al., 2009; Benamar et al., 2016). In addition, the DTL gene, encoding CDT2, is amplified in a subset of Ewing carcinoma (Mackintosh et al., 2012). Using mRNA expression in public databases of HNSCC (Ginos et al., 2004; Sengupta et al., 2006; Pyeon et al., 2007; Peng et al., 2011), it was determined that CDT2 mRNA expression is elevated in oropharyngeal carcinoma (4.636-fold compared to normal squamous mucosa of the oral cavity; p=6.5×10⁻⁵; t-test=7.121) and in nasopharyngeal carcinoma (5.487-fold compared to nasopharynx; p=1.04×10⁻¹⁰; i-test=10.181). CDT2 ranks in the top 3% in oropharyngeal SCC and in the top 1% in nasopharyngeal carcinoma of overexpressed mRNAs in these arrays. CDT2 was also overexpressed in other HNSCCs (Ginos et al., 2004; Pyeon et al., 2007; Peng et al., 2011), including oral cavity carcinoma (2.475-fold compared to normal tissue; p=1.9×10⁻⁹; t-test=8.985), tonsillar carcinoma (2.422-fold compared to normal tissue; p=3.46×10; t-test=5.863), and floor of mouth carcinoma (4.801-fold compared to normal tissue; p=3.37×10⁻⁶; t-test=9.610). CDT2 overexpression in hepatocellular carcinoma, gastric cancer, and melanoma is associated with poor overall and disease-free survival (Pan et al., 2006; Kobayashi et al., 2015; Benamar et al., 2016).

To test whether elevated CDT2 expression in HNSCC correlated with patient survival, CDT2 expression (based on RNA-seq) was stratified in two large data sets of HNSCC tumors available through The Cancer Genome Atlas TCGA databases (Cerami et al., 2012; Gao et al., 2013) into high-CDT2 expressors (CDT2 level >0.2z) and low-CDT2 expressors (CDT2 level <0.2z). No statistically significant correlation between CDT2 expression and overall survival (high-expressors: n=272, median of 56.9 months vs. low expressors: n=245, median of 56.44 months; p=0.883) or disease-free survival (high-expressors: n=81, median of 26.41 months vs. low expressors: n=78, median of 21.49 months; p=0.893) was found, leading to the conclusion that CDT2 overexpression in HNSCC was not predictive of patient outcome.

### Example 9

### CDT2 Depletion in HNSCC Cells Induced Robust Rereplication and Inhibited Proliferation

Next, whether CDT2 was essential for the proliferation or viability of HNSCC cell lines was tested. The expression of CDT2 was silenced in two HPV-ve HNSCC cell lines, Cal27 and FaDu, using a previously validated siRNA (Abbas et al., 2008). These two lines were selected because they were extensively profiled and were found to harbor some of the most commonly found mutations in head and neck cancers, including inactivating mutations in genes encoding p53, p16, and NOTCH2/3 receptors (Agrawal et al., 2011; Stransky et al., 2011; Nichols et al., 2012). CDT2 depletion in either of these cell lines resulted in a significant increase in the levels of CRL4^{CDT2} substrates p21 and SET8. However, a significant increase in CDT1 protein in Cal27 or in FaDu cells was not detected, presumably because CDT1 ubiquitylation and degradation in S-phase is additionally mediated via the activity of SCF^{SKP2} E3 ubiquitin ligase following its phosphorylation by cyclin A/CDK2 (Li et al., 2003; Liu et al., 2004; Nishitani et al., 2006).

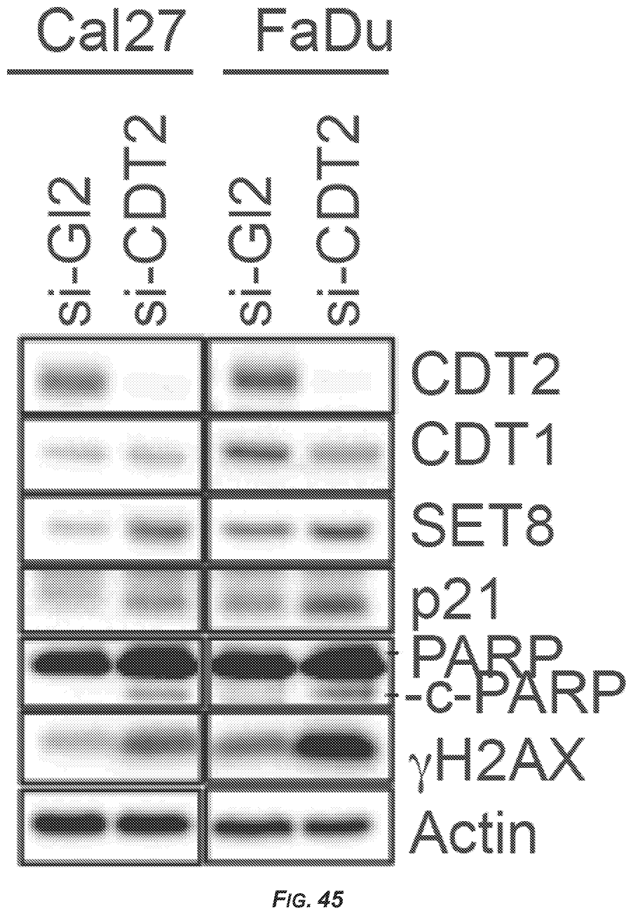

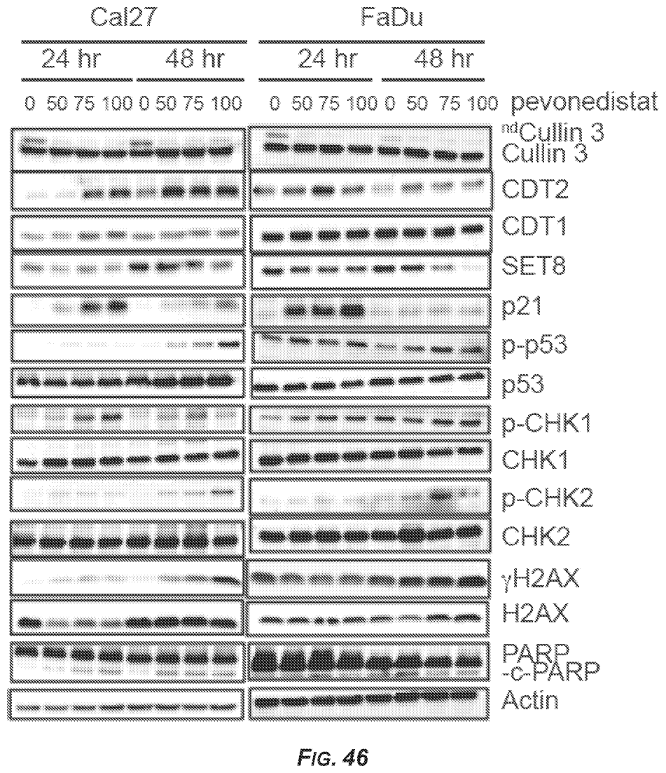

Consistent with the role of CDT2 in suppressing genome stability, transient silencing of CDT2 led to the accumulation of spontanous DNA damage as manifested by the induction of γH2AX and increased phosphorylation of the checkpoint kinases CHK1 and CHK2 (FIG. 45). Importantly, CDT2 depletion inhibited the proliferation of Cal27 and FaDu cells. Inhibition of cell proliferation in CDT2-depleted cells was accompanied by significant morphological changes commonly seen in cells undergoing DNA rereplication: flattening of cells and increased nuclear and cytoplasmic volume. Consistently, FACS analysis of Cal27 or FaDu cells depleted of CDT2 demonstrated significant increase in cells with greater than 4N DNA content, with 55.7% of Cal27 cells (vs. 1.17% absent CDT2 depletion) and 43.9% of FaDu cells (vs. 3.52% absent CDT2 depletion) undergoing rereplication. BrdU labeling confirmed that DNA rereplication occurred within the same cell cycle. A substantial increase in cells with sub-G1 DNA content was not detected, consistent with the observation that only a minor increase in cleaved PARP protein was detected in cells depleted of CDT2 (FIG. 45).

Furthermore, the depletion of CDT2 in these two cell lines was not associated with cell senescence, as beta-galactosidase staining was not detected in these cells. This was likely due to mutations in CDKN2A (encoding p16), which as disclosed herein are critical for rereplication-induced senescence (Benamar et al., 2016). These results suggested that neither apoptosis nor senescence significantly contributed to proliferation inhibition following CDT2 depletion.

Collectively, the instant results demonstrated that CDT2 played an important role in promoting the proliferation of HNSCC and was important in preventing DNA rereplication and the accumulation of DNA damage.

### Example 10

### Pevonedistat Inhibited HNSCC Cell Proliferation Through Induction of Rereplication

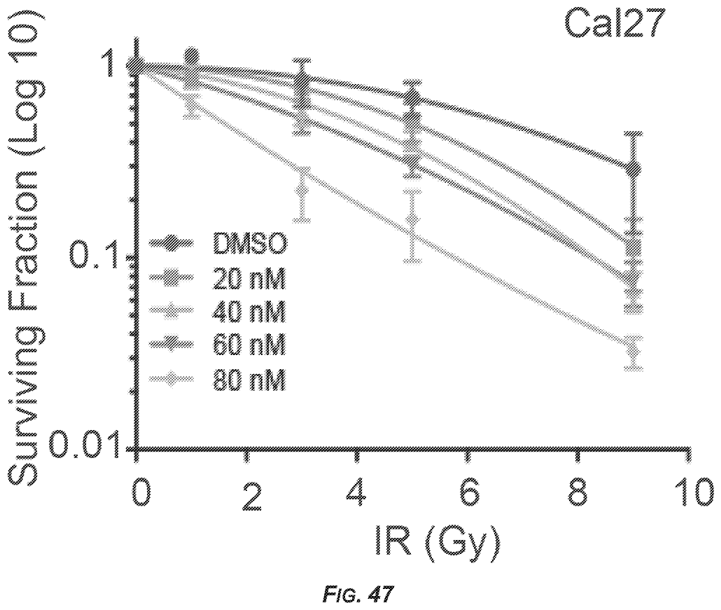

Pevonedistat was shown to induce rereplication in a variety of cancer cell lines (Soucy et al., 2009). Furthermore, the presently disclosed subject matter demonstrated that pevonedistat-induced rereplication and growth inhibition in melanoma cells is dependent on CRL4^{CDT2} inhibition and the resultant increased stability of p21 and SET8 proteins (see also Benamar et al., 2016). Thus, the impact of pevonedistat on the proliferation of HPV-ve HNSCC cells was tested. Cal27 or FaDu cells were treated with increasing concentrations of pevonedistat. Treatment of either cell line with 50 nM pevonedistat inhibited cullin neddylation for 24 or 48 hours and was accompanied by increased levels of the CRL4^{CDT2} substrate p21 (FIG. 46). In contrast, CDT1 and SET8 were induced at early time points but returned to normal levels by 24 hours in both Cal27 or FaDu cells.

Furthermore, pevonedistat treatment resulted in the accumulation of DNA damage in Cal27 as manifested by γH2AX, and was accompanied by activation of the G2/M checkpoint, as evident by increased phophorylated CHK1 and CHK2 kinases (FIG. 46). Similar to cells depleted of CDT2, pevonidestat treatment of Cal27 or FaDu cells did not result in significant apoptosis as only a minor increase in cleaved PARP protein was detected (FIG. 46). Exposure of Cal27 and FaDu cells to pevonedistat inhibited the proliferation of both cell lines, as determined by cell counting. Colony formation and CYQUANT® brand cell viability assays (Promega Corporation; Madison, Wis., United States of America) demonstrated that pevonedistat inhibited the proliferation of HNSCC cells with an IC₅₀ of approximately 50 nM, a concentration that was comparable to that seen in the most sensitive melanoma cell lines (see EXAMPLE 4 above; see also Benamar et al., 2016).

Additionally, only transient treatment of Cal27 with pevonedistat (100 nM) for 24 hours was sufficient to permanently halt cell proliferation. FACS analysis by propidium iodide (PI) staining showed that pevonedistat induced robust rereplication with the majority of Cal27 (61%) or FaDu (60.4%) cells treated with 100 nM pevonedisat exhibiting >4N DNA content.

Rereplication was also observed with 40 nM concentration as early as 24 hours following treatment. On the other hand, only 5-10% of the cells exhibited sub-G1 DNA content (indicative of apoptosis) when examined at 72 hours post-treatment. Importantly, DNA rereplication was not observed in control hTERT-transformed keratonocytes (OKF6-TERT2), consistent with the notion discussed herein that normal cells have additional mechanisms to suppress DNA rereplication (see also Benamar et al., 2016).

### Example 11

### Pevonedistat Sensitized HNSCC Cells to Ionizing Radiation In Vitro

HPV-ve HNSCC cells and tumors are extremely resistant to IR (Lassen et al., 2009; Kotowski et al., 2011; O'Sullivan et al., 2012; Kimple et al., 2013; Rieckmann et al., 2013; Sorensen et al., 2013; Arenz et al., 2014). Recent studies demonstrated that pevonedistat enhances the sensitivity of pancreatic, breast and colorectal cancer cells to IR (Wei et al., 2012; Yang et al., 2012; Wan et al., 2016). Thus, whether pevonedistat sensitized Cal27 or FaDu HNSCC cells to IR was tested by measuring cell survival following the incubation of cells with increasing doses of pevonedistat using standard colony formation assays.

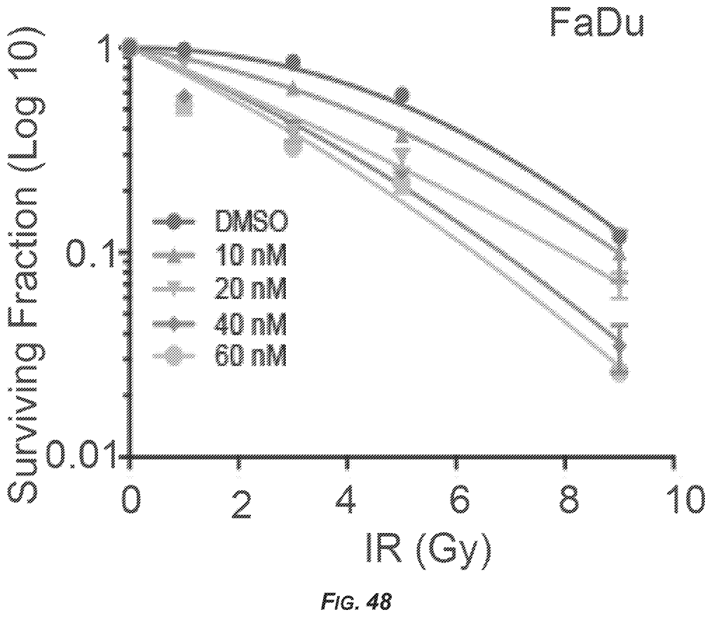

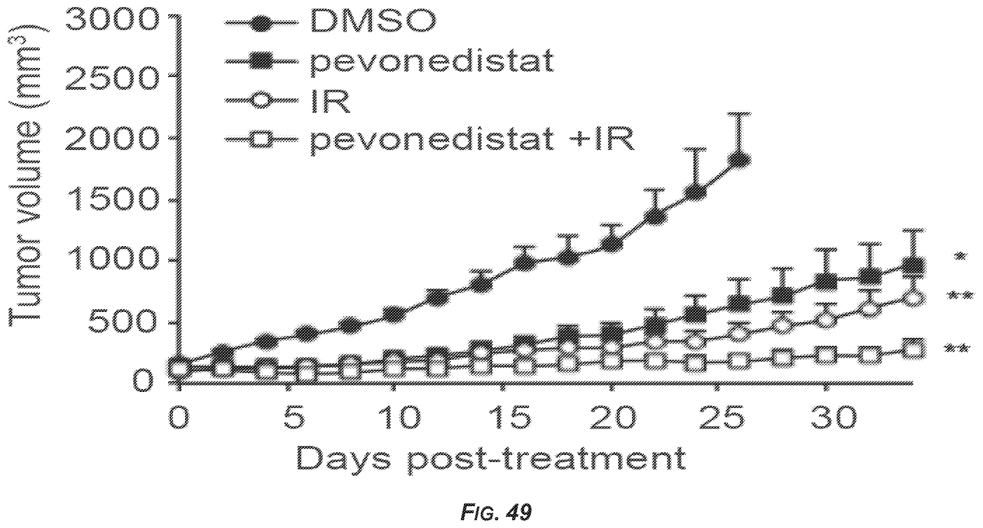

First, both Cal27 and FaDu cells were confirmed to be significantly resistant to IR, with only 81% and 88.4% of the cells losing replicative potential with 9 Gy of IR (see FIGS. 47 and 48). Pre-treatment of Cal27 with increasing doses of pevonedistat for 24 hours significantly and dose-dependently enhanced their sensitivity to radiation, with sensitivity enhancement ratios (SER) of 2.99 when measured at 10% survival and following 80 nM pevonedistat treatment (FIG. 47 and Table 10).

TABLE 10
Pevonedistat Enhanced Radiation
Sensitivity of HPV-ve HNSCC Cells

| | | | | | | |
|---|---|---|---|---|---|---|
| [Pevonedistat] | | | | | | |
| (nM) | α | β | S | SF4 | SER | p Value |

Cal27

| | | | | | | |
|---|---|---|---|---|---|---|
| DMSO | 2.03 × 10⁻¹⁶ | 0.035 | 0.1 | 8.074 | 1 | <0.0001 |
| 20 | 1.50 × 10⁻¹³ | 0.049 | 0.1 | 6.893 | 1.171 | <0.0001 |
| 40 | 0.108 | 0.052 | 0.1 | 5.692 | 1.419 | <0.0001 |
| 60 | 0.471 | 0.009 | 0.1 | 4.457 | 1.812 | <0.0001 |
| 80 | 0.906 | −0.020 | 0.1 | 2.702 | 2.702 | <0.0001 |

FaDu

| | | | | | | |
|---|---|---|---|---|---|---|
| DMSO | 1.62 × 10⁻¹⁶ | 0.023 | 0.1 | 9.3471 | 1 | <0.0001 |
| 10 | 0.105 | 0.017 | 0.1 | 8.979 | 1.055 | <0.0001 |
| 20 | 0.242 | 0.006 | 0.1 | 7.987 | 1.186 | <0.0001 |
| 40 | 0.241 | 0.014 | 0.1 | 6.830 | 1.387 | <0.0001 |
| 60 | 0.282 | 0.013 | 0.1 | 6.328 | 1.497 | <0.0001 |

Pevonedistat similarly radiosensitized FaDu cells, albeit to a lower extent, with SER of 1.49 when measured at 10% survival and following 60 nM pevonedistat treatment (FIG. 48 and Table 10). It is important to note that higher doses of pevonedistat in either line were associated with complete suppression of proliferation, precluding any further assessment of radiosensitization at these higher doses. Pevonedistat also radiosensitized other HPV-ve HNSCC lines, including SCC25 and UNC7 cells.

Pevonedistat-induced radiosensitizing activity in breast and colon cancer cells was attributed to the induction of G2/M cell cycle arrest (Yang et al., 2012; Wan et al., 2016). In pancreatic cells however, pevonedistat induced rereplication, which was stimulated by IR (Wei et al, 2012). To understand the mechanistic basis of pevonedistat-enhanced radiation sensitivity in HNSCC cells, the cell cycle distribution of Cal27 or FaDu cells exposed to IR was tested with or without pevonedistat treatment. As expected, exposure of these cells to 4 Gy IR failed to upregulate p21 protein due to inactivating mutations in the gene coding for the p53 tumor suppressor protein. Consistently, exposure of Cal27 or FaDu cells to 2 or 4 Gy IR did not result in G1 growth arrest and instead was accompanied by G2 cell cycle arrest.

However, an increase in the number of Cal27, and more prominently FaDu cells, with polyploid nuclei was evident. As expected, treatment of Cal27 or FaDu cells with 40 nM pevonedistat for 48 hours resulted in 16.8% and 21.6% of the cells undergoing rereplication, respectively. Importantly, exposure of these cells at the 24 hour time point to 2 Gy resulted in significantly higher percentage of cells undergoing rereplication (32.9% (p<0.01) and 42.2% (p<0.05), respectively), with further increased percentages at a higher dose of 4 Gy (4 Gy vs. 2 Gy: Cal27: p<0.05 and FaDu p<0.01). Increased DNA rereplication by the combined treatment resulted in a small, but reproducible, increase in γH2AX, indicating the accumulation of more DNA damage.

### Example 12

### Pevonedistat Suppresses HNSCC Xenografts and Synergizes with IR to Suppress HNSCC in Nude Mice

The results above suggested that pevonedistat suppressed HNSCC cells through the induction of DNA rereplication and this additionally elicits radiosensitizing activity. To test if pevonedistat exhibits anti-cancer and/or radiosensitizing activities in HNSCC tumors, nude mice were inoculating with Cal27 cells and tumor growth was monitored. A randomized group of mice were treated with DMSO or with 20 mg/kg pevonedistat (IP) for 5 consecutive days for two cycles separated by 5 days of no treatment similar to previously reported in vivo murine studies with this drug (Soucy et al., 2009). A third group of animals received 1 Gy of radiation daily, and a fourth group received both treatments. Animals were weighed and monitored daily and the drug was well tolerated. Pevonedistat significantly suppressed Cal27 xenografts (p=0.0211). Moreover, although IR suppressed these xenografts (p=5.5×10⁻³ vs. control), its combined treatment with pevonedistat resulted in more suppression than either treatment alone (p=2×10⁻³ vs. control; see FIG. 49).

### Example 13

### Induction of Rereplication Via CDT2 Depletion or CDT1 Activation Radiosensitized HNSCC Cells

Pevonedistat inhibits all cullin-based E3 ligases and additionally exhibits cullin-independent activity (Soucy et al., 2009; Lin et al., 2010; Zhao et al., 2011; Gu et al., 2014; Godbersen et al., 2014; Li et al., 2014a; Li et al., 2014b). However, the results described above suggested that the radiosensitizing activity of pevonedistat was due to its ability to induce rereplication, which can be enhanced by IR. To test whether rereplication was sufficient to confer radiosensitivity in HNSCC cells, the expression of CDT2 was silenced in Cal27 or FaDu cells by si-CDT2 for 72 hours to inactivate the CRL4^{CDT2} ligase and carried out clonogenic survival assays. CDT2 depletion in Cal27 cells enhanced radiation sensitivity with an SER of 1.34 when measured at 10% survival (Table 11). As expected, silencing of CDT2 or exposure of CDT2-proficient cells to 4 Gy IR resulted in the accumulation of DNA damage (γH2AX) and activation of and G2/M checkpoints (phosphorylated CHK1 and CHK2 protein), and this was augmented by the combined treatment. Furthermore, and similar to pevonedistat treatment, low doses of IR increased the percentage of CDT2-depleted Cal27 cells undergoing rereplication. Similar results were obtained in FaDu cells, although CDT2 depletion was less radiosensitizing in this cell line.

TABLE 11
Depletion of HNSCC Cells of CDT2, Geminin,
or CDT1 Radiosensitized HPV-ve HNSCC Cells

| | | | | | | |
|---|---|---|---|---|---|---|
| Condition | α | β | s | SF4 | SER | p Value |

Cal27

| | | | | | | |
|---|---|---|---|---|---|---|
| si-GL2 | 1.817 × 10⁻¹⁶ | 0.021 | 0.1 | 10.449 | 1 | <0.0001 |
| si-CDT2 | 0.110 | 0.024 | 0.1 | 7.790 | 1.341 | <0.0001 |
| si-geminin | 0.114 | 0.048 | 0.1 | 5.852 | 1.786 | <0.0001 |
| Si-EMI1 | 0.147 | 0.024 | 0.1 | 7.225 | 1.446 | <0.0001 |

FaDu (Experiment A)

| | | | | | | |
|---|---|---|---|---|---|---|
| si-GL2 | 0.096 | 0.003 | 0.1 | 16.061 | 1 | <0.0001 |
| si-geminin | 0.192 | 0.004 | 0.1 | 10.072 | 1.595 | <0.0001 |
| Si-EMI1 | 0.145 | 0.006 | 0.1 | 10.773 | 1.492 | <0.0001 |

FaDu (Experiment B)

| | | | | | | |
|---|---|---|---|---|---|---|
| si-GL2 | 2.15 × 10⁻¹³ | 0.024 | 0.1 | 9.848 | 1 | <0.0001 |
| si-CDT2 | 0.078 | 0.017 | 0.1 | 9.508 | 1.036 | <0.0001 |

To test whether rereplication was sufficient to confer radiation sensitivity in HNSCC in cells with intact CRL4^{CDT2} activity, Cal27 or FaDu cells were depleted of geminin, an endogenous inhibitor of CDT1 protein known to induce rereplication in some cancer cells (Zhu & Depamphilis, 2009; Benamar et al., 2016). Consistently, depletion of Cal27 or FaDu cells of geminin induced rereplication. Importantly, geminin silencing prior to IR exposure induced robust radiation sensitivity in both lines with an SER of 1.79 and 1.59, respectively (Table 11). Furthermore, in Cal27 and in FaDu cells, silencing of EMI1, an inhibitor of the APC/C ubiquitin ligase whose depletion induces rereplication (Machida & Dutta, 2007), similarly induced rereplication, and sensitized both lines to IR with an SER of 1.45 and 1.49, respectively (4 Gy IR vs. si-EMI alone: p<0.01 for Cal27 and p<0.001 for FaDu; see also Table 11).

As was the case in cells treated with pevonedistat or depleted of CDT2, cells depleted of geminin or EMI1 and exposed to 4 Gy IR exhibited more DNA damage and cell cycle checkpoint activation than cells depleted of these two proteins or exposed to IR. In addition and similar to pevonedistat treatment or CRL4^{CDT2} inactivation, low doses of IR (2 and 4 Gy) greatly increased the percentage of rereplicating Cal27 or FaDu cells depleted of geminin (4 Gy IR vs. si-geminin alone: p<0.001 for both Cal27 and FaDu) or EMI1 (4 Gy IR vs. si-EMI alone: p<0.01 for Cal27 and p<0.001 for FaDu).

Collectively, these results demonstrated that induction of DNA rereplication was sufficient to confer radiosensitivity in HNSCC cells, and that exposure of cancer cells to low or moderate doses of radiation rendered them more susceptible to rereplication, presumably by inducing cell cycle block in late S and G2 phases of the cell cycle.

### DISCUSSION OF THE EXAMPLES

Disclosed herein is the demonstration that CDT2 is frequently overexpressed in melanoma, and its elevated expression predicts poor overall and disease-free survival. CDT2 knockdown or deletion inhibits the proliferation of melanoma cells in vitro through the induction of DNA rereplication and senescence, and via a mechanism that is dependent on the stabilization of the CRL4^{CDT2} substrates SET8 and p21. Pevonedistat exerts significant and BRAF-independent anti-melanoma activity through the induction of DNA rereplication and senescence, both of which require p21 and SET8. In vivo studies using melanoma cells with hypomorphic expression of p21 or SET8 show that both of these proteins are required for the efficiency of pevonedistat to suppress melanoma, demonstrating that CRL4^{CDT2} inhibition represents the primary mechanism of pevonedistat-mediated anti-melanoma activity. Also shown is that pevonedistat synergizes with vemurafenib to suppress BRAF mutant melanoma in vivo, and suppresses vemurafenib-resistant melanoma cells.

The CRL4^{CDT2} E3 ubiquitin ligase was identified as a molecular therapeutic target in melanoma. CDT2 knockdown or the pharmacological inhibition of CRL4^{CDT2} activity by the neddylation inhibitor pevonedistat inhibits melanoma cell proliferation in vitro and in vivo through the induction of SET8- and p21-dependent aberrant DNA replication and the induction of p21-dependent cellular senescence. Although p16 can also be involved in rereplication-induced senescence, it is not essential for pevonedistat-induced toxicity, and thus, mutational inactivation of the CDKN2A or oncogenic activation of BRAF or NRAS, all common genetic defects in melanoma, do not present an obstacle for the therapeutic efficacy of pevonedistat. Pevonedistat was also efficacious in suppressing melanoma cells that are resistant to vemurafenib treatment in vitro and synergized with vemurafenib to suppress mutant BRAF melanoma. Furthermore, because pevonedistat still induces rereplication and inhibits melanoma cells that resisted vemurafenib treatment, the results disclosed herein demonstrated that pevonedistat can be effective as a second line therapeutic for vemurafenib-relapsed melanoma patients.

Mechanistically, CRL4^{CDT2} has been shown herein to be the primary target of inactivation by pevonedistat in melanoma, and its toxicity has been shown to be dependent primarily on the stabilization of the CRL4^{CDT2} substrates SET8 and p21 both in vitro and in vivo. Disclosed herein is solid genetic evidence that the main anti-melanoma activity of pevonedistat was associated with its ability to promote DNA rereplication and permanent growth arrest that was dependent on the stabilization of the CRL4^{CDT2} substrates SET8 and p21. Although all the three CRL4^{CDT2} substrates CDT1, SET8 and p21 were independently required to induce rereplication and senescence in melanoma cells with inactivated CRL4^{CDT2}, only SET8 was both necessary and sufficient to promote rereplication and the ensuing senescence.

The exact mechanism by which deregulated SET8 expression promoted rereplication is currently unclear, but methylation of histone H4K20 might be critical for this activity (Abbas et al., 2010; Tardat et al., 2010). The main role of p21 on the other hand, appeared to halt cell cycle progression, thus permitting rereplication, and induced senescence. This was supported by the finding that p21 was critical for the induction of rereplication and senescence in response to CRL4^{CDT2} inactivation (by si-CDT2 or pevonedistat), and was also upregulated in rereplicating cells following the ectopic expression of CDT1 or SET8^{ΔPIP}, or following EMI1 depletion, but was insufficient to induce rereplication.

Although CDT1 clearly promotes rereplication in other cancer cells as demonstrated by the robust induction of rereplication through geminin depletion, such a role was not observed in melanoma cells likely because CDT1 activity is restrained by cyclin A-dependent SCF^{SKP2} activity. Non-physiological overexpression of CDT1 however, was sufficient to induce rereplication in melanoma cells, but this is likely to also require SET8 and p21. This conclusion is supported by the fact that although pevonedistat induced significant rereplication in melanoma cells, it failed to do so in cells with hypomorphic expression of SET8 or p21, despite significant increases in CDT1 protein (FIGS. 32 and 39). The anti-melanoma activity of pevonedistat and its dependence on CRL4^{CDT2} inhibition and the induction of SET8- and p21-dependent rereplication provide a stronger link between presumed drug target (NAE) and biology than is available for many other “targeted therapies”.

The study disclosed herein also demonstrated that CDT2 was significantly overexpressed in melanoma, and its elevated expression correlated significantly with poor overall and disease-free patient survival. Because elevated CDT2 expression correlated with, and renders melanoma cells more susceptible to, pevonedistat-induced rereplication in vitro, and given that rereplication appeared to play a major role in mediating its efficacy in vivo, pevonedistat could be most efficacious in tumors with elevated CDT2 expression. This includes not only melanoma, but potentially other malignancies with elevated CDT2.

Although it is unlikely that CDT2 functions as a classical oncogene, it appeared to act as a cancer-associated gene to which cancer cells become “addicted”. This is reminiscent to the secondary physiological changes that stress cellular capacity for survival as a consequence of oncogenic activation, common in melanoma and in other cancers; the so called “non-oncogene addiction” (Luo et al., 2009). This is supported by the finding that while CRL4^{CDT2} inactivation by pevonedistat induces rereplication in melanoma cells, it failed to do so in non-cancer melanocytic cells. Similarly, CDT2 depletion in non-cancer cells failed to induce rereplication in non-cancer cells, but did so following the ectopic expression of KRAS (Olivero et al., 2014). While it is not desired to be bound by any particular theory of operation, it is thus proposed that CDT2 is overexpressed in melanoma cells to alleviate replication stress that maybe induced by melanoma oncogenes.

Because only transient exposure of melanoma cells to pevonedistat is sufficient to irreversibly arrest melanoma cells with the majority of cells undergoing cell senescence, and this occurs only in malignant melanoma cells, targeting the CRL4^{CDT2} ubiquitin ligase is an especially attractive therapeutic approach that is likely to be associated with only minimal impact on normal cellular activity or cytotoxicity. Why non-malignant melanocytes exposed to pevonedistat are only transiently inhibited, and without undergoing robust rereplication, is not clear, but the results disclosed herein support the hypothesis that non-cancer cells have additional mechanisms to guard against aberrant DNA rereplication (Abbas et al., 2013).

The CRL4 substrate receptor CDT2 is overexpressed in various human cancers (see e.g., Benamar et al., 2016). The presently disclosed subject matter extends this observation to HNSCC, where CDT2 is found to be significantly overexpressed in HNSCC from various tissue origins. Unlike the case for hepatocellular carcinoma, gastric cancer, or melanoma, where CDT2 expression is correlated with poor patient outcome (see e.g., Benamar et al., 2016), elevated CDT2 expression in head and neck cancers did not correlate with patient outcome. Thus, although CDT2 was essential for the proliferation of HNSCC cancer cells, it was not likely to be involved in HNSCC tumor progression. Instead, and given that CDT2 depletion in HNSCC cells was associated with DNA rereplication and the accumulation of DNA damage, CDT2 appeared to be critical for coping with the replication stress in these highly proliferative cells, functioning as a cancer-associated gene, which was critical for the capacity of cells to survive the consequences of oncogenic transformation; the so called “non-oncogene addiction” (Luo et al., 2009).

As disclosed herein, pevonedistat suppressed melanoma in vitro and in vivo through the induction of DNA rereplication downstream of CRL4^{CDT2} inhibition and the stabilization of its ubiquitylation substrates SET8 and p21 (see also Benamar et al., 2016). Pevonedistat also suppressed the proliferation of HPV-ve HNSCC cells and tumors, and this was similarly due to the induction of robust rereplication. In melanoma cells, the main cytotoxicity associated with pevonedistat-induced rereplication appeared to be the induction of senescence, which correlated with the presence of functional p16 tumor suppressor protein (Benamar et al., 2016). However, senescence was note detected in Cal27 or in FaDu cells, presumably because they both lack functional p16 protein and additionally have inactivating mutations in the p53 tumor suppressor proteins (Agrawal et al., 2011; Stransky et al., 2011; Nichols et al. (2012).

Furthermore, pevonedistat-induced growth inhibition in Cal27 or in FaDu cells was not associated with apoptosis. The induction of rereplication and lack of apoptotic response in Cal27 or in FaDu cells treated with low pevonedistat concentrations (20-100 nM) was in contrast to the robust apoptosis observed in a previous study (Zhao et al., 2011) in SqCC/Y1 or in Trl46 HNSCC cells (55 and 65% of cells) treated with 5-10 fold higher doses of pevonedistat (0.5 and 1 μM, respectively). Such apoptotic induction however, was attributed to the induction of c-FLIP degradation, and this was independent of NEDD8 inhibition (Zhao et al., 2011). Thus, pevonedistat, might exhibit differential toxicities in HNSCC cells and tumors depending on the genetic backgrounds of the cells or tumors as well as on the concentrations employed.

Also disclosed herein is that low doses of pevonedistat significantly radiosensitized HPV-ve HNSCC cells. Pevonedistat also synergized with IR to suppress HNSCC tumorigenesis in nude mice. In vitro studies demonstrated that low doses of IR greatly and synergistically increased the percentage of cells undergoing rereplication in response to low doses of pevonedistat. The finding that induction of rereplication, through direct inactivation of CRL4^{CDT2} or through CDT1 activation or stabilization in cells with intact CRL4^{CDT2} activity, also radiosensitized HNSCC cells strongly supported the conclusion that the main radiosensitizing activity of pevonedistat was mediated through its rereplication-inducing activity.

The results also demonstrated that induction of rereplication was sufficient to confer radiosensitizing activity in HNSCC cells. Other cancers that exhibit elevated levels of radiation resistance could respond similarly.

Finally, because pevonedistat does not induce rereplication in non-malignant keratinocytes, and underlies its radiosensitizing activity, the results disclosed herein also suggested that pevonedistat-induced radiosensitization might be selective to cancer cells over normal tissue, which would be desirable to reducing treatment-related toxicity in HNSCC.

As such, the present disclosure supports the fact that the CRL4^{CDT2} ubiquitin ligase represents a novel molecular target for inhibition and radiosensitization in HPV-ve HNSCC. the present disclosure also demonstrates that pevonedistat exhibited promising anti-tumor and radiosensitizing activity in HNSCC, and that induction of rereplication represents a novel therapeutic strategy for radiosensitization.

Summarily, the CRL4^{CDT2} ubiquitin ligase is emerging as a master regulator of cell proliferation. Shown herein is that CDT2 was overexpressed in cutaneous melanoma and predicted poor overall and disease-free survival. Knockdown or knockout of CDT2 inhibited a panel of melanoma cell lines through the induction of SET8- and p21-dependent DNA rereplication and senescence. In vitro and in vivo studies demonstrated that pevonedistat (MLN4924), an inhibitor of protein neddylation, was effective at inhibiting melanoma through the stabilization of the CRL4^{CDT2} substrates p21 and SET8. Pevonedistat additionally synergized with vemurafenib to inhibit BRAF melanoma, and suppressed vemurafenib-resistant melanoma cells. The findings disclosed herein indicated that targeting the CRL4^{CDT2} ligase has a promising anti-melanoma potential, and demonstrated that a broad patient population could benefit from pevonedistat therapy.

### REFERENCES

All references listed in the instant disclosure, including but not limited to all patents, patent applications and publications thereof, scientific journal articles, and database entries (including but not limited to GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Abbas & Dutta (2011) CRL4Cdt2: master coordinator of cell cycle progression and genome stability. Cell Cycle 10:241-249.
Abbas et al. (2008). PCNA-dependent regulation of p21 ubiquitylation and degradation via the CRL4Cdt2 ubiquitin ligase complex. Genes Dev 22:2496-2506.
Abbas et al. (2010). CRL4(Cdt2) regulates cell proliferation and histone gene expression by targeting PR-Set7/Set8 for degradation. Mol Cell 40:9-21.
Abbas et al. (2013) Genomic instability in cancer. Cold Spring Harb Perspect Biol 5:a012914.
Agrawal et al. (2011) Exome sequencing of head and neck squamous cell carcinoma reveals inactivating mutations in NOTCH1. Science 333:1154-1157.
Altschul & Lipman (1990) Protein database searches for multiple alignments. Proc Natl Acad Sci USA 87:14:5509-5513.
Altschul et al. (1990) Basic local alignment search tool. J Mol Biol 215:403-410.
Altschul et al. (1997) Gapped BLAST and PSI-BLAST: a new generation of protein database search programs. Nucl Acid Res 25:3389-3402.
Arenz et al. (2014) Increased radiosensitivity of HPV-positive head and neck cancer cell lines due to cell cycle dysregulation and induction of apoptosis. Strahlenther Onkol 190:839-846.
Balch et al. (2009). Final version of 2009 AJCC melanoma staging and classification. J Clin Oncol 27:6199-6206.
Beck et al. (2012) The role of PR-Set7 in replication licensing depends on Suv4-20h. Genes Dev 26:2580-2589.
Benamar et al. (2016) Inactivation of the CRL4-CDT2-SET8/p21 ubiquitylation and degradation axis underlies the therapeutic efficacy of pevonedistat in melanoma. EBioMedicine 10:85-100.
Bird et al. (1988) Single-chain antigen-binding proteins. Science 242:423-426.
Bliss (1939). The toxicity of poisons applied jointly. Ann Appl Biol 26:585-615.
Bliss, C., 1939. The toxicity of poisons applied jointly. Ann Appl Biol 26:585-215.
Bodanszky & Bodanszky (1984) in The Practice of Peptide Synthesis, Springer-Verlag, New York, N.Y., United States of America.
Cargnello & Roux (2011) Activation and function of the MAPKs and their substrates, the MAPK-activated protein kinases. Microbiol Mol Biol Rev 75:50-83.
Cerami et al. (2012). The cBio cancer genomics portal: an open platform for exploring multidimensional cancer genomics data. Cancer Discov 2:401-404.
Chapman et al. (2011). Improved survival with vemurafenib in melanoma with BRAF V600E mutation. New Eng J Med 364:2507-2516.
Chemical Abstract Service (CAS) Registry Nos. 905579-51-3; 1029872-54-5.
Chiba & Tanaka (2004) Cullin-based ubiquitin ligase and its control by NEDD8-conjugating system. Curr Prot Pep Sci 5:177-184.
Chou & Fasman (1974) Prediction of protein conformation. Biochemistry 13:222-245.
Chou & Fasman (1978) Empirical predictions of protein conformation. Ann Rev Biochem 47:251-276.
Chou & Fasman (1979) Prediction of beta-turns. Biophys J 26:367-384.
Collisson et al. (2014) Comprehensive molecular profiling of lung adenocarcinoma. Nature 511:543-550.
Czuczman et al. (2015) Pevonedistat, a NEDD8 activating enzyme inhibitor, is active in mantle cell lymphoma and enhances rituximab activity in vivo. Blood 127: 1128-1137.
D'Errico et al. (2009) Genome-wide expression profile of sporadic gastric cancers with microsatellite instability. Eur J Cancer 45:461-469.
Davies & Samuels (2010). Analysis of the genome to personalize therapy for melanoma. Oncogene 29:5545-5555.
Davies et al. (2002). Mutations of the BRAF gene in human cancer. Nature 417:949-954.
Deutscher et al. (ed.) (1990) Guide to Protein Purification, Harcourt Brace Jovanovich, San Diego, Calif., United States of America.
Devereux et al. (1984) A comprehensive set of sequence analysis programs for the VAX. Nucl Acids Res 12:387.
Erdag et al. (2012) Immunotype and immunohistologic characteristics of tumor-infiltrating immune cells are associated with clinical outcome in metastatic melanoma. Cancer Res 72:1070-1080.
Fitzgerald et al. (2006). Systems biology and combination therapy in the quest for clinical efficacy. Nat Chem Biol 2:458-466.
Flaherty et al. (2010). Inhibition of mutated, activated BRAF in metastatic melanoma. New Eng J Med 363:809-819.
Gao et al. (2013). Integrative analysis of complex cancer genomics and clinical profiles using the cBioPortal. Sci Signal 6:pl1.
Garcia et al. (2014). Nedd8-activating enzyme inhibitor MLN4924 provides synergy with mitomycin C through interactions with ATR, BRCA1/BRCA2, and chromatin dynamics pathways. Mol Cancer Ther 13:1625-1635.
Gerhardt et al. (eds.) (1994) Methods for General and Molecular Bacteriology, American Society for Microbiology, Washington, D.C., United States of America, page 574.
Ginos et al. (2004) Identification of a gene expression signature associated with recurrent disease in squamous cell carcinoma of the head and neck. Cancer Res 64:55-63.
Glickman & Ciechanover (2002). The ubiquitin-proteasome proteolytic pathway: destruction for the sake of construction. Physiol Rev 82:373-428.
Godbersen et al. (2014) The Nedd8-activating enzyme inhibitor MLN4924 thwarts microenvironment-driven NF-kappaB activation and induces apoptosis in chronic lymphocytic leukemia B cells. Clin Cancer Res 20:1576-1589.
Gong & Yeh (1999) Identification of the activating and conjugating enzymes of the NEDD8 conjugation pathway. J Biol Chem 274: 12036-12042.
Gross & Mienhofer (eds.) (1981) The Peptides, Vol. 3, Academic Press, New York, N.Y., United States of America, pages 3-88.
Gu et al. (2014). MLN4924, an NAE inhibitor, suppresses AKT and mTOR signaling via upregulation of REDD1 in human myeloma cells. Blood 123:3269-3276.
Harlow et al. (1989) Antibodies: A Laboratory Manual, Cold Spring Harbor, N.Y., United States of America.
Harlow et al. (1999) Using Antibodies: A Laboratory Manual, Cold Spring Harbor Laboratory Press, Cold Spring Harbor, N.Y., United States of America.
Hersey et al. (2009). Small molecules and targeted therapies in distant metastatic disease. Ann Oncol 20 Suppl 6:vi35-40.
Hogan et al. (2005). Use of selected reaction monitoring mass spectrometry for the detection of specific MHC class I peptide antigens on A3 supertype family members. Cancer Immunol Immunother C1154:359-371.
Hou et al. (2010) Gene expression-based classification of non-small cell lung carcinomas and survival prediction. PloS One 5:e10312.
Huntington et al. (2004). Overexpression of collagenase 1 (MMP-1) is mediated by the ERK pathway in invasive melanoma cells: role of BRAF mutation and fibroblast growth factor signaling. J Biol Chem 279:33168-33176.
Huston et al. (1988) Protein engineering of antibody binding sites: recovery of specific activity in an anti-digoxin single-chain Fv analogue produced in Escherichia coli. Proc Natl Acad Sci USA 85:5879-5883.
Hutvagner & Zamore (2002) A microRNA in a multiple-turnover RNAi enzyme complex. Science 297:2056-2060.
Jackson & Xiong (2009). CRL4s: the CUL4-RING E3 ubiquitin ligases. Trends Biochem Sci 34:562-570.
Jazaeri et al. (2013). Overcoming platinum resistance in preclinical models of ovarian cancer using the neddylation inhibitor MLN4924. Mol Cancer Ther 12:1958-1967.
Jin et al. (2006). A family of diverse Cul4-Ddb-interacting proteins includes Cdt2, which is required for S phase destruction of the replication factor Cdt1. Mol Cell 23:709-721.
Jinek et al. (2012) A programmable dual-RNA-guided DNA endonuclease in adaptive bacterial immunity. Science 337:816-821.
Karlin & Altschul (1990) Methods for assessing the statistical significance of molecular sequence features by using general scoring schemes. Proc Natl Acad Sci USA 87:2264-2268.
Karlin & Altschul (1993) Applications and statistics for multiple high-scoring segments in molecular sequences. Proc Natl Acad Sci USA 90:5873-5877.
Ketting et al. (2001) Dicer Functions in RNA Interference and in Synthesis of Small RNA Involved in Developmental Timing in C. elegans. Genes Dev 15:2654-2659.
Kimple et al. (2013) Enhanced radiation sensitivity in HPV-positive head and neck cancer. Cancer Res 73:4791-4800.
Kittlesen et al. (1998). Human melanoma patients recognize an HLA-A1-restricted CTL epitope from tyrosinase containing two cysteine residues: implications for tumor vaccine development. J Immunol 160:2099-2106.
Kobayashi et al. (2015) Overexpression of denticleless E3 ubiquitin protein ligase homolog (DTL) is related to poor outcome in gastric carcinoma. Oncotarget 6:36615-36624.
Kotowski et al. (2011) Radiosensitization of head and neck cancer cells by the phytochemical agent sulforaphane. Strahlenther Onkol 187:575-580.
Kyte & Doolittle (1982) A simple method for displaying the hydropathic character of a protein, J Mol Biol 157:105-132.
Lassen et al. (2009) Effect of HPV associated p16INK4A expression on response to radiotherapy and survival in squamous cell carcinoma of the head and neck. J Clin Oncol 27:1992-1998.
Le Poole et al. (2000). PIG3V, an immortalized human vitiligo melanocyte cell line, expresses dilated endoplasmic reticulum. In Vitro Cell Dev Biol Anim 36:309-319.
Li et al. (2003) The SCF(Skp2) ubiquitin ligase complex interacts with the human replication licensing factor Cdt1 and regulates Cdt1 degradation. J Biol Chem 278:30854-30858.
Li et al. (2009) Identification of retinoic acid-regulated nuclear matrix-associated protein as a novel regulator of gastric cancer. Br J Cancer 101:691-698.
Li et al. (2014a). Inactivation of SAG/RBX2 E3 ubiquitin ligase suppresses KrasG12D-driven lung tumorigenesis. J Clin Ivest 124:835-846.
Li et al. (2014b). Overactivated neddylation pathway as a therapeutic target in lung cancer. J Natl Cancer Inst 106:dju083.
Lin et al. (2010). NEDD8-targeting drug MLN4924 elicits DNA rereplication by stabilizing Cdt1 in S phase, triggering checkpoint activation, apoptosis, and senescence in cancer cells. Cancer Res 70:10310-10320.
Liu et al. (2004) Cyclin-dependent kinases phosphorylate human Cdt1 and induce its degradation. J Biol Chem 279:17283-17288.
Lovly & Shaw (2014). Molecular pathways: resistance to kinase inhibitors and implications for therapeutic strategies. Clin Cancer Res 20:2249-2256.
Luo et al. (2009). Principles of cancer therapy: oncogene and non-oncogene addiction. Cell 136:823-837.
Machida & Dutta (2007). The APC/C inhibitor, Emil, is essential for prevention of rereplication. Genes Dev 21:184-194.
Mackintosh et al. (2012) 1q gain and CDT2 overexpression underlie an aggressive and highly proliferative form of Ewing sarcoma. Oncogene 31:1287-1298.
Martinez et al. (2002) Single-Stranded Antisense siRNAs Guide Target RNA Cleavage in RNAi. Cell 110:563-574.
Merlet et al. (2009). Regulation of cullin-RING E3 ubiquitin-ligases by neddylation and dimerization. Cell Mol Life Sci 66:1924-1938.
Milhollen et al. (2010). MLN4924, a NEDD8-activating enzyme inhibitor, is active in diffuse large B-cell lymphoma models: rationale for treatment of NF-{kappa}B-dependent lymphoma. Blood 116:1515-1523.
Milhollen et al. (2011). Inhibition of NEDD8-activating enzyme induces rereplication and apoptosis in human tumor cells consistent with deregulating CDT1 turnover. Cancer Res 71:3042-3051.
Molhoek et al. (2008). Human melanoma cytolysis by combined inhibition of mammalian target of rapamycin and vascular endothelial growth factor/vascular endothelial growth factor receptor-2. Cancer Res 68:4392-4397.
Nakagawa et al. (2008) Transcriptional regulation of an evolutionary conserved intergenic region of CDT2-INTS7. PLoS One 3:e1484.
Nichols et al. (2012) Frequent mutations in TP53 and CDKN2A found by next-generation sequencing of head and neck cancer cell lines. Arch Otolaryngol Head Neck Surg 138:732-739.
Nishitani et al. (2006) Two E3 ubiquitin ligases, SCF-Skp2 and DDB1-Cul4, target human Cdt1 for proteolysis. EMBO J 25:1126-1136.
O'Sullivan et al. (2012) Outcomes of HPV-related oropharyngeal cancer patients treated by radiotherapy alone using altered fractionation. Radiother Oncol 103:49-56.
Oda et al. (2010). Regulation of the histone H4 monomethylase PR-Set7 by CRL4(Cdt2)-mediated PCNA-dependent degradation during DNA damage. Mol Cell 40:364-376.
Olivero et al. (2014). The stress phenotype makes cancer cells addicted to CDT2, a substrate receptor of the CRL4 ubiquitin ligase. Oncotarget 5:5992-6002.
Pan et al. (2006) Role of L2DTL, cell cycle-regulated nuclear and centrosome protein, in aggressive hepatocellular carcinoma. Cell Cycle 5:2676-2687.
Peng et al. (2011) A novel molecular signature identified by systems genetics approach predicts prognosis in oral squamous cell carcinoma. PLoS One 6:e23452.
Petroski & Deshaies (2005) Function and regulation of cullin-RING ubiquitin ligases. Nature Rev Mol Cell Biol 6:9-20.
Plotnikov et al. (2011) The MAPK cascades: signaling components, nuclear roles and mechanisms of nuclear translocation. Biochim Biophys Aca 1813:1619-1633.
Provost et al. (2002) Ribonuclease Activity and RNA Binding of Recombinant Human Dicer. EMBO J 21:5864-5874.
Pyeon et al. (2007) Fundamental differences in cell cycle deregulation in human papillomavirus-positive and human papillomavirus-negative head/neck and cervical cancers. Cancer Res 67:4605-4619.
Read et al. (2000) Nedd8 modification of cul-1 activates SCF(beta(TrCP))-dependent ubiquitination of IkappaBalpha. Mol Cell Biol 20:2326-2333.
Remington's Pharmaceutical Science. 18th ed. (1990) Mack Publishing, Easton, Pa., United States of America.
Rieckmann et al. (2013) HNSCC cell lines positive for HPV and p16 possess higher cellular radiosensitivity due to an impaired DSB repair capacity. Radiother Oncol 107:242-246.
Roessler et al. (2010) A unique metastasis gene signature enables prediction of tumor relapse in early-stage hepatocellular carcinoma patients. Cancer Res 70:10202-10212.
Roller et al. (2015). Combinatorial drug screening and molecular profiling reveal diverse mechanisms of intrinsic and adaptive resistance to BRAF inhibition in V600E BRAF mutant melanomas. Oncolarget 7:2734-2753.
Romano et al. (2013) Identification of multiple mechanisms of resistance to vemurafenib in a patient with BRAFV600E-mutated cutaneous melanoma successfully rechallenged after progression. Clin Cancer Res 19:5749-5757.
Schotta et al. (2008). A chromatin-wide transition to H4K20 monomethylation impairs genome integrity and programmed DNA rearrangements in the mouse. Genes Dev 22:2048-2061.
Senga et al. (2006). PCNA is a cofactor for Cdt1 degradation by CUL4/DDB1-mediated N-terminal ubiquitination. J Biol Chem 281:6246-6252.
Sengupta et al. (2006) Genome-wide expression profiling reveals EBV-associated inhibition of MHC class I expression in nasopharyngeal carcinoma. Cancer Res 66:7999-8006.
Siegel et al. (2012). Cancer treatment and survivorship statistics, 2012. Cancer J Clin 62:220-241.
Slingluff et al. (1993). Recognition of human melanoma cells by HLA-A2.1-restricted cytotoxic T lymphocytes is mediated by at least six shared peptide epitopes. J Immunol 150:2955-2963.
Solus & Kraft (2013). Ras, Raf, and MAP kinase in melanoma. Adv Anat Pathol 20:217-226.
Sorensen et al. (2013) Radiosensitivity and effect of hypoxia in HPV positive head and neck cancer cells. Radiother Oncol 108:500-505.
Sosman et al. (2012). Survival in BRAF V600-mutant advanced melanoma treated with vemurafenib. New Eng J Med 366:707-714.
Soucy et al. (2009). An inhibitor of NEDD8-activating enzyme as a new approach to treat cancer. Nature 458:732-736.
Stewart et al. (1984) in Solid Phase Peptide Synthesis, 2nd Edition, Pierce Chemical Company, Rockford, Ill., United States of America.
Stransky et al. (2011) The mutational landscape of head and neck squamous cell carcinoma. Science 333:1157-1160.
Sun et al. (2006) Neuronal and glioma-derived stem cell factor induces angiogenesis within the brain. Cancer Cell 9:287-300.
Tabara et al. (2002) The dsRNA Binding Protein RDE-4 Interacts with RDE-1, DCR-1 and a DexH-box Helicase to Direct RNAi in C. elegans. Cell 109:861-871.
Talantov et al. (2005). Novel genes associated with malignant melanoma but not benign melanocytic lesions. Clin Cancer Res 11:7234-7242.
Tan et al. (2013). Endothelial deletion of Sag/Rbx2/Roc2 E3 ubiquitin ligase causes embryonic lethality and blocks tumor angiogenesis. Oncogene 33:5211-5220.
Tardat et al. (2010). The histone H4 Lys 20 methyltransferase PR-Set7 regulates replication origins in mammalian cells. Nat Cell Biol 12:1086-1093.
Taylor et al. (2008) Apoptosis: controlled demolition at the cellular level. Nat Rev Mol Cell Biol 9:231-241.
Taylor et al. (2010) Integrative genomic profiling of human prostate cancer. Cancer Cell 18:11-22.
Truong & Wu (2011) Prevention of DNA re-replication in eukaryotic cells. J Mol Cell Biol 3:13-22.
Tsai et al. (2008). Discovery of a selective inhibitor of oncogenic B-Raf kinase with potent antimelanoma activity. Proc Natl Acad Sci USA 105:3041-3046.
U.S. Pat. Nos. 6,734,283; 7,691,997; 9,260,752; 9,410,198.
Ueki et al. (2008) Involvement of elevated expression of multiple cell-cycle regulator, DTL/RAMP (denticleless/RA-regulated nuclear matrix associated protein), in the growth of breast cancer cells. Oncogene 27:5672-5683.
Wan et al. (2016) Radiosensitization of Human Colorectal Cancer Cells by MLN4924: An Inhibitor of NEDD8-Activating Enzyme. Technol Cancer Res Treat 15:527-534.
Wei et al. (2012). Radiosensitization of human pancreatic cancer cells by MLN4924, an investigational NEDD8-activating enzyme inhibitor. Cancer Res 72, 282-293.
Weinstein et al. (2014) Comprehensive molecular characterization of urothelial bladder carcinoma. Nature 507:315-322.
Yamshchikov et al. (2001). Analysis of a natural immune response against tumor antigens in a melanoma survivor: lessons applicable to clinical trial evaluations. Clin Cancer Res 7:909s-916s.
Yamshchikov et al. (2005). Sequential immune escape and shifting of T cell responses in a long-term survivor of melanoma. J Immunol 174:6863-6871.
Yang et al. (2012) The p21-dependent radiosensitization of human breast cancer cells by MLN4924, an investigational inhibitor of NEDD8 activating enzyme. PLoS One 7:e34079.
Zhai et al. (2007) Gene expression analysis of preinvasive and invasive cervical squamous cell carcinomas identifies HOXC10 as a key mediator of invasion. Cancer Res 67:10163-10172.
Zhao et al. (2011) The NEDD8-activating enzyme inhibitor, MLN4924, cooperates with TRAIL to augment apoptosis through facilitating c-FLIP degradation in head and neckcancer cells. Mol Cancer Ther 10:2415-2425.
Zhu & Depamphilis (2009). Selective killing of cancer cells by suppression of geminin activity. Cancer Res 69:4870-4877.

It will be understood that various details of the presently disclosed subject matter can be changed without departing from the scope of the presently disclosed subject matter. Furthermore, the foregoing description is for the purpose of illustration only, and not for the purpose of limitation.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. A method for treating a cancer, optionally wherein the cancer is selected from the group consisting of melanoma, breast cancer, head and neck squamous carcinoma cell (HNSCC) cancer, a solid tumor, hepatocellular carcinoma, colorectal cancer, non-small-cell lung cancer, serous ovarian cancer, papillary thyroid carcinoma, and ameloblastoma, the method comprising administering to a subject in need thereof a composition comprising an effective amount of a composition comprising an effective amount of an inhibitor of a cullin-based CRL4^{CDT2} ubiquitin ligase biological activity, optionally wherein the inhibitor of the cullin-based CRL4^{CDT2} ubiquitin ligase biological activity is an inhibitor of a NEDD8 activating enzyme (NAE).

27. The method of claim 26, wherein the composition comprises pevonedistat or a pharmaceutically acceptable salt and/or solvate thereof, and vemurafenib or a pharmaceutically acceptable salt and/or solvate thereof.

28. (canceled)

29. (canceled)

30. (canceled)

31. A method for overcoming vemurafenib-resistance in a cell, the method comprising contacting a vemurafenib-resistant cell with an effective amount of an inhibitor of cullin-based CRL4^{CDT2} ubiquitin ligase biological activity, optionally wherein the inhibitor of cullin-based CRL4^{CDT2} ubiquitin ligase biological activity comprises pevonedistat, a pharmaceutically acceptable salt and/or solvate thereof, or any combination thereof.

32. The method of claim 31, wherein the cell is a tumor cell and/or a cancer cell, optionally a melanoma cell or a colorectal cell.

33. (canceled)

34. (canceled)

35. A method for treating a tumor or a cancer in a subject, the method comprising:
identifying a subject having a tumor or a cancer associated with CDT2 overexpression;
administering to the subject a therapeutic agent comprising a composition comprising an effective amount of an inhibitor of a cullin-based CRL4^{CDT2} ubiquitin ligase biological activity, optionally wherein the inhibitor of the cullin-based CRL4^{CDT2} ubiquitin ligase biological activity is an inhibitor of a NEDD8 activating enzyme (NAE); and
administering to the subject a radiation therapy before, during, and/or after administering to the subject the therapeutic agent comprising the composition.

36. The method of claim 35, wherein the tumor or the cancer is selected from the group consisting of melanoma, breast cancer, head and neck squamous carcinoma cell (HNSCC) cancer, a solid tumor, hepatocellular carcinoma, colorectal cancer, a non-small-cell lung cancer, serous ovarian cancer, papillary thyroid carcinoma, and ameloblastoma.

37. The method of claim 26, further comprising treating the subject with at least one additional anti-cancer therapy, optionally wherein the at least one additional anti-cancer therapy is selected from the group consisting of radiotherapy, chemotherapy, immunotherapy, surgery, and combinations thereof.

38. The method of claim 37, wherein the at least one additional anti-cancer therapy comprises administering vemurafenib, dabrafenib, trametinib, cobimetinib, a pharmaceutically acceptable salt and/or solvate thereof, or any combination thereof to the subject.

39. The method of claim 37, wherein the at least one additional anti-cancer therapy comprises administering vemurafenib, a pharmaceutically acceptable salt and/or solvate thereof, or any combination thereof, to the subject in need thereof.

40. The method of claim 37, wherein the at least one additional anti-cancer therapy comprises administering ipilimumab, pembrolizumab, nivolumab, interleukin-2 (IL-2), a pharmaceutically acceptable salt and/or solvate thereof, or any combination thereof, or any combination thereof to the subject.

41. The method of claim 37, wherein the at least one additional anti-cancer therapy comprises administering to the subject at least one second therapeutic agent selected from the group consisting of a BRAF inhibitor, an MEK inhibitor, an anti-CRL4^{CDT2} ubiquitin ligase inhibitory nucleic acid, an anti-p21 inhibitory nucleic acid, an anti-CDT1 inhibitory nucleic acid, an anti-SET8 inhibitory nucleic acid, an anti-geminin inhibitory nucleic acid, an anti-CDKN1A inhibitory nucleic acid, an anti-EMI1 inhibitory nucleic acid, or any combination thereof.

42. The method of claim 41, wherein:
(a) the BRAF inhibitor is selected from the group consisting of vemurafenib or a pharmaceutically acceptable salt and/or solvate thereof, dabrafenib or a pharmaceutically acceptable salt and/or solvate thereof, and sorafenib or a pharmaceutically acceptable salt and/or solvate thereof, or any combination thereof; and/or
(b) the MEK inhibitor is trametinib or a pharmaceutically acceptable salt and/or solvate thereof, or any combination thereof; and/or
(c) the anti-CRL4^{CDT2} ubiquitin ligase inhibitory nucleic acid comprises SEQ ID NO: 2, SEQ ID NO: 15, or SEQ ID NO: 16; and/or
(d) the anti-p21 inhibitory nucleic acid comprises SEQ ID NO: 5; and/or
(e) the anti-CDT1 inhibitory nucleic acid comprises SEQ ID NO: 3; and/or
(f) the anti-SET8 inhibitory nucleic acid comprises SEQ ID NO: 4 or SEQ ID NO: 17; and/or
(g) the anti-geminin inhibitory nucleic acid comprises SEQ ID NO: 6; and/or
(h) the anti-CDKN1A inhibitory nucleic acid comprises SEQ ID NO: 19; and/or
(i) the anti-EMI1 inhibitory nucleic acid comprises SEQ ID NO: 7 or SEQ ID NO: 8.

43. The method of claim 37, wherein the at least one additional anti-cancer therapy is administered to the subject in a separate composition.

44. The method of claim 37, wherein the composition and the at least one additional anti-cancer therapy are present in the same composition.

45. (canceled)

46. (canceled)


SEQUENCE LISTING (18 sequences)

SEQ ID NO: 1 - RNA, 21 nt, Artificial Sequence
  Artificially synthesized oligonucleotide
aacguacgcggaauacuucga

SEQ ID NO: 2 - RNA, 19 nt, Artificial Sequence
  Artificially synthesized oligonucleotide
gaauuauacugcuuaucga

SEQ ID NO: 3 - RNA, 21 nt, Artificial Sequence
  Artificially synthesized oligonucleotide
aacguggaugaaguacccgac

SEQ ID NO: 4 - RNA, 19 nt, Artificial Sequence
  Artificially synthesized oligonucleotide
gauugaaagugggaaggaa

SEQ ID NO: 5 - RNA, 19 nt, Artificial Sequence
  Artificially synthesized oligonucleotide
aacauacuggccuggacug

SEQ ID NO: 6 - RNA, 19 nt, Artificial Sequence
  Artificially synthesized oligonucleotide
ugccaacucuggaaucaaa

SEQ ID NO: 7 - RNA, 21 nt, Artificial Sequence
  Artificially synthesized oligonucleotide
gagaauuucggugacagucua

SEQ ID NO: 8 - RNA, 21 nt, Artificial Sequence
  Artificially synthesized oligonucleotide
uacgaagugucucuguaauua

SEQ ID NO: 9 - DNA, 22 bp, Artificial Sequence
  Artificially synthesized oligonucleotide
tgttgtgagaggcgcaagctgc

SEQ ID NO: 10 - DNA, 22 bp, Artificial Sequence
  Artificially synthesized oligonucleotide
ggtcggaggtggcgtgtgtttc

SEQ ID NO: 11 - DNA, 22 bp, Artificial Sequence
  Artificially synthesized oligonucleotide
gtctttcccccacctccgcctg

SEQ ID NO: 12 - DNA, 23 bp, Artificial Sequence
  Artificially synthesized oligonucleotide
cttttttcggggggcctgtttgc

SEQ ID NO: 13 - DNA, 24 bp, Artificial Sequence
  Artificially synthesized oligonucleotide
tcacctgaggtgacacagcaaagc

SEQ ID NO: 14 - DNA, 23 bp, Artificial Sequence
  Artificially synthesized oligonucleotide
ggccccgtgggaaggtagagctt

SEQ ID NO: 15 - DNA, 20 bp, Artificial Sequence
  Artificially synthesized oligonucleotide
gcaccgaattgaagagcatc

SEQ ID NO: 16 - DNA, 20 bp, Artificial Sequence
  Artificially synthesized oligonucleotide
catttctcaggacgccaagc

SEQ ID NO: 17 - DNA, 20 bp, Artificial Sequence
  Artificially synthesized oligonucleotide
acggagcgccatgaagtccg

SEQ ID NO: 18 - DNA, 20 bp, Artificial Sequence
  Artificially synthesized oligonucleotide
gcgccatgtcagaaccggct